(19) United States
(12) Patent Application Publication
(10) Pub. No.: US 2016/0184459 A1
(43) Pub. Date: Jun. 30, 2016

(54) CANCER CELL SPECIFIC IMAGING PROBES AND METHODS OF USE

(71) Applicant: THE RESEARCH FOUNDATION FOR THE STATE UNIVERSITY OF NEW YORK, Albany, NY (US)

(72) Inventors: Nobuhide Ueki, Forest Hills, NY (US); Michael J. Hayman, Patchogue, NY (US)

(73) Assignee: The Research Foundation for the State University of New York, Albany, NY (US)

(21) Appl. No.: 14/911,187

(22) PCT Filed: Aug. 7, 2014
(86) PCT No.: PCT/US2014/050190

§ 371 Date: Feb. 9, 2016

Related U.S. Application Data
(60) Provisional application No. 61/864,112, filed on Aug. 9, 2013.

(51) Int. Cl. A61K 49/00; A61K 51/04; C07H 19/16; A61K 49/10; C07H 23/00; C07H 21/04

(52) U.S. Cl. CPC A61K 49/0004; C07H 23/00; C07H 21/04; C07H 19/16; A61K 49/108; A61K 51/0491; A61K 49/0058; A61K 49/0032

(57) ABSTRACT

The present invention provides a compound having the structure:

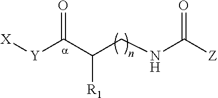

wherein
X is an imaging agent containing at least one amine nitrogen;
Y is a chemical linker,
   wherein Y is present or absent, and when present Y is a chemical linker containing at least one amine nitrogen or Y is a para-aminobenzyl alcohol linker;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

This application claims priority of U.S. Provisional Application No. 61/864,112, filed Aug. 9, 2013, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

This invention was made with government support under grant number CA009176 awarded by the National Institutes of Health. The government has certain rights in the invention

### BACKGROUND OF THE INVENTION

There is a growing interest in developing therapeutic agents with increased selectivity. The ability of a therapeutic agent to affect a particular population of cells in preference over others is a highly desirable property. A therapeutic agent or drug having low selectivity leads to reduced efficacy and higher toxicity. For example, a major limitation of many cancers treatments is their low selectivity for tumor cells. Radiation therapy and alkylating agents perturb many functions that are common to both tumor and normal cells.

HDACs are key enzymes involved in the epigenetic regulation of histone and non-histone proteins (Witt, O. et al. 2009). They modulate protein structure and function through deacetylation of lysine residues. In cancer biology, the involvement of HDACs has been well documented, supporting the notion that altered expression of HDACs could have an active role in tumor development (Haberland, M. et al. 2009; Bolden, J. E. et al. 2006). Consistent with this, the therapeutic potential of HDAC inhibitors (HDACi) is recognized as a new class of drug for cancer (Bolden, J. E. et al. 2006; Minucci, S. et al. 2006; Marks, P. A. & Xu, W. S. 2009). HDAC inhibitors (HDACi), which were developed as single target agents, are a new class of drugs for cancer (Minucci, S. & Pelicci, P. G. 2006; Bolden, J. E. et al. 2006; Marks, P. A. & Xu, W. S. 2009). Currently a number of HDACi are in clinical trials for various hematologic and solid tumors (Marks, P. A. & Xu, W. S. 2009; Wagner, J. M. et al. 20104). In preclinical studies, several HDACi have been found to have potent anticancer effects. However, adverse side effects have been reported in a number of preclinical trials (Bolden, J. E. et al. 2006; Wagner, J. M. et al. 2010). Therefore, selectivity remains a major challenge. Also, since certain HDACs are essential for normal cells, a single target agent using these pharmacologic inhibitors depends on the tolerance levels of normal cells to the damage caused by the treatment (Lee, J. H. et al. 2010; Bhaskara, S. et al. 2010).

Tumor-associated cysteine protease CTSL also plays crucial roles at multiple stages of tumor progression and metastasis (Joyce, J. A. et al. 2004; Jedeszko, C. et al. 2004; Gonzalez-Suarez, I. et al. 2011).

Cell lines transformed by certain oncogenes including Ras are known to express high levels of CTSL (Collette, J. et al. 2004; Denhardt, D. T. et al. 1987; Joseph, L. J. et al. 1988). Thus, the upregulation of CTSL is recognized as a hallmark of metastatic cancers and could be utilized as a prognostic marker (Joyce, J. A. et al. 2004; Jedeszko, C. et al. 2004; Gonzalez-Suarez, I. et al. 2011; Tian, Y, et al. 2011; Grotsky, D. A. et al. 2013). Recently, nuclear-localized CTSL involved in cancer has been revealed, suggesting that CTSL may have key roles in the nucleus beyond its known lysosomal and extracellular activities (Gonzalez-Suarez, I. et al. 2011; Grotsky, D. A. et al. 2013; Goulet, B. et al. 2004; Goulet, B. et al. 2007). Although the therapeutic potential of CTSL inhibitors has not been fully characterized in preclinical studies, targeting CTSL activity is considered as a strategy for anticancer therapy (Lankelma, J. M. et al. 2010).

Therefore, drugs with improved selectivity are still urgently needed to combat cancer and various other diseases. Such selectivity allows for a drug with maximal efficacy and minimal adverse effects or toxicity.

The problem of selectivity is also encountered when using imaging techniques to diagnose cancer. Imaging cancer cells in vivo has important implications for detecting and diagnosing cancers and monitoring therapy. However, the conventional technologies are limited in part due to a low tumor/normal tissue signal to background ratio. Known imaging techniques include x-ray, ultrasound, magnetic resonance imaging (MRI), single-photon emission computed tomography (SPECT), positron emission tomography (PET) and optical imaging (Frangioni, J. V. et al. 2008).

Magnetic resonance imaging (MRI) imaging involves the use of high magnetic fields and radio-frequency signals to produce images. MRI uses a magnetic field to align the magnetization of protons in the body followed by radio frequency pulses that are absorbed by the protons. As the protons relax back into alignment with the magnetic field a signal is received by a radio frequency coil and a computer constructs an image of the scanned area of the body. MRI contrast agents are used to improve the visibility of the targeted regions of the body. Positron Emission Tomography (PET) involves detection of pairs of gamma rays emitted indirectly by a positron-emitting radionuclide (tracer) placed in the body. The PET tracer is introduced into the body on a biologically relevant molecule. Images of tracer concentration in the body are then reconstructed by computer analysis. Optical imaging takes advantage of the ability of tissue to absorb photons. In fluorescence optical tomography, a fluorophore is injected into the body and the tissue is illuminated. A part of the light which is absorbed by the fluorophore is re-emitted as a longer wavelength and reconstructed by computer analysis to form an image. ¹⁸F-FDG radiopharmaceuticals for PET are most widely used in clinics to detect cancers (Gallagher et al. 1977). Although ¹⁸F-FDG with PET has proven its efficacy in general oncologic imaging, its uptake is not specific to cancers. Since the ¹⁸F-FDG uptake relies on the metabolic status of the cells, certain normal cells including actively metabolizing cells and inflammatory cells also show increased uptake of 18F-FDG, making difficult to distinguish cancer cells from normal cells in some cases.

Therefore, imaging probes (i.e., MRI contrast agents, PET imaging agents or optical imaging agents) with improved selectivity for cancer cells relative to normal cells are still urgently needed to detect cancer cells. Such probes would allow for specific visualization of cancer cells within the body and have a significant effect on early cancer detection and monitoring of tumors in response to therapy.

### SUMMARY OF THE INVENTION

The present invention provides a compound having the structure:

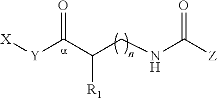

wherein
X is an imaging agent containing at least one amine nitrogen;
Y is a chemical linker,
   wherein Y is present or absent, and when present Y is a chemical linker containing at least one amine nitrogen or Y is a para-aminobenzyl alcohol linker;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
wherein
when Y is absent, an amine nitrogen on the imaging agent covalently bonds directly to carbon α, or
when Y is present and is a chemical linker containing at least one amine nitrogen, an amine nitrogen on the linker covalently bonds directly to carbon α, or
when Y is present and is a para-aminobenzyl alcohol linker, the nitrogen on the linker Y bonds directly to carbon α and the oxygen on the linker Y connects to the imaging agent X, or the oxygen on the linker Y bonds directly to carbon α and the nitrogen on the linker Y connects to the imaging agent X through an amide bond;
   or a diastereomer, enantiomer or pharmaceutically acceptable salt of the compound.

The present invention also provides a method of imaging cancer cells in a subject comprising:
1) administering to the subject an effective amount of a compound having the structure:

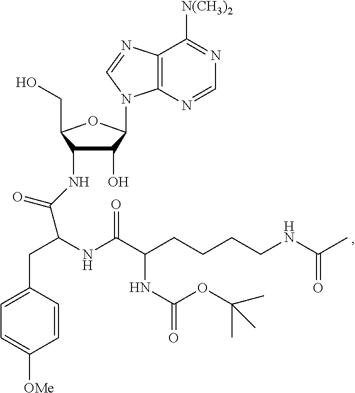

or a pharmaceutically acceptable salt thereof,
   wherein the compound specifically accumulates at cancer cells in the subject and releases puromycin;
2) administering to the subject an amount of an antibody conjugated to a detectable marker, which antibody is capable of specifically binding to the puromycin at the cancer cells in the subject;
3) detecting in the subject the location of the detectable marker; and
4) obtaining an image of the cancer cells in the subject based on the location of the detectable marker in the subject.

### BRIEF DESCRIPTION OF THE FIGURES

FIG. 1. Comparative HDAC activity of the panel of cancer and normal cell lines measured by a standard HDAC assay using substrate Boc-Lys(Ac)-AMC either with vehicle control DMSO or TSA (1 μM). Data represent mean values of triplicate measurements±s.d. RFU, relative fluorescent units.

FIG. 2. Comparative live cell lysyl endopeptidase activity of the same cell lines as in FIG. 1 using substrate Boc-Lys-AMC either with vehicle control DMSO or Z-FY-CHO (100 μM). Data represent mean values of triplicate measurements±s.d. RFU, relative fluorescent units.

FIG. 3. Prostate cancer cells exhibit high enzymatic activity to convert Boc-Lys(Ac)-AMC releasing AMC in live cells. Comparative live cell enzymatic activity. Prostate cancer (PC-3, DU-145, and LNCaP), colon cancer (HCT116 as a positive control sensitive to the drug), and normal colon (CCD841-CoN as a negative control) cells were analyzed using substrate Boc-Lys(Ac)-AMC either with vehicle control DMSO, TSA (1 μM) or Z-FY-CHO (100 μM). Data represent mean values of triplicate measurements±s.d. RFU, relative fluorescent units.

FIG. 4. Comparative live cell enzymatic activity of the same cell lines as in FIG. 1 using substrate Boc-Lys(Ac)-AMC either with vehicle control DMSO, TSA (1 μM) or Z-FY-CHO (100 μM). Data represents mean values of triplicate measurements±s.d. RFU, relative fluorescent units.

FIG. 5. A. HDAC assay using different substrates in BxPC3 cells (shGFP and shSki). Cells were incubated with Boc-Lys(Ac)-AMC or Ac-Arg-Gly-Lys(Ac)-AMC (25 μM) for 1 hr with DMSO (vehicle) or TSA (1 μM) as indicated, followed by cell lysis and trypsin treatment, and fluorescence measurements. Data represent mean values of triplicate measurements±standard deviation. RFU, relative fluorescent units. B. Similar experiment was done as in A but without cell lysis and trypsin treatment. Cells were incubated with the substrates for 2.5 hr.

FIG. 6. A. Release of AMC by combined HDAC and endogenous protease-dependent enzymatic reaction in BxPC3 and CFPac-1 cells (shGFP and shSki). Cells were incubated with substrate Boc-Lys(Ac)-AMC (25 μM) for 6 hr with DMSO (vehicle), TSA (1 μM), or Z-FY-CHO (10 μM) as indicated, followed by fluorescence measurements. Data represent mean values of triplicate measurements±standard deviation. RFU, relative fluorescent units. B. Time course of AMC release in BxPC3 cells (shGFP and shSki). Each cells were incubated with Boc-Lys(Ac)-AMC (25 μM) for 2.5, 6.0, and 23 hr with DMSO (vehicle) or TSA (1 μM) as indicated.

FIG. 7. A scheme of the selective two-step drug activation in cancer cells by HDAC and CTSL.

FIG. 8. Boc-Lys(Ac)-Puromycin, Spectral data for Boc-KAc-Puro. (a) ¹H NMR (500 MHz, DMSO-d6). (b) ¹³C NMR (101 MHz, DMSO-d6).

FIG. 9. The colon cancer cell lines were grown for 5 d either with vehicle control DMSO (gray symbols) or 54 μM BKAc-Puro (red and blue symbols), and cell number was determined at the indicated time points.

FIG. 10. The cell lines were grown as in FIG. 8, cell viability was determined by trypan blue at the indicated time points, and is presented as the percentage of live cells treated with vehicle control (gray symbols) or BKAc-Puro (red and blue symbols).

FIG. 11. The cell lines were grown as in FIG. 8, and dead cells were determined after 80 h by PI signal under fluorescent microscope. BF, bright field. PI, fluorescent PI channel.

FIG. 12. Selective cytotoxicity by BKAc-Puro. The cell lines were grown and analyzed as in FIG. 10. BF, bright field. PI, fluorescent PI channel.

FIG. 13. Inhibition of cell viability by BKAc-Puro is presented in dose-response curve format for the same cell lines as in FIG. 8. The cell lines were treated with DMSO or the indicated doses of agent (4.22, 8.44, 16.9, 33.8, 67.5, or 135 μM) for 5 d followed by MTS assay. Data represent mean values of triplicate measurements±s.d. (n=3). IC₅₀ values were fit by logistic regression.

FIG. 14. Inhibition of cell viability by parental Puro. The cell lines were treated with the indicated doses of Puro (0, 1.1, 2.1, 4.2, or 8.4 μM) for 3 d followed by MTS assay. Data represent mean values of triplicate measurements±s.d.

FIG. 15. Inhibition of cell viability by BKAc-Puro. The cell lines were grown and analyzed as in FIG. 12.

FIG. 16. Anticancer effect of BKAc-Puro on pancreatic cell lines. The cell lines were treated with DMSO (vehicle), BKAc-Puro (54 μM), or Puro (4.2 μM) for 5 d followed by MTS assay. Normal Eph4 (non-pancreatic) cells are shown as control. Data represent mean values of triplicate measurements±s.d.

FIG. 17. Selective cytotoxicity by BKAc-Puro on breast cancer cells. Normal mammary gland epithelial cells (Eph4) and breast cancer cells (MCF-7 and MDA-MB-231) were grown and analyzed as in FIG. 10. BF, bright field. PI, fluorescent PI channel. BKAc-Puro can selectively cause cell death in breast cancer cells (MCF-7 and MDA-MB-231) while leaving normal Eph4 cells unharmed.

FIG. 18. Selective cytotoxicity by BKAc-Puro on pancreatic cancer cells. Pancreatic cancer (BXPC-3 and Miapaca-2), colon cancer (HCT116 as a positive control sensitive to the drug), and normal colon (CCD841-CoN as a negative control) cells were grown and analyzed as in FIG. 13. BKAc-Puro can effectively cause cell death on pancreatic cancer cells (BXPC-3 and MiaPaca-2) that are known to resistant to conventional chemotherapeutic drugs including 5-FU and Gemicitabine.

FIG. 19. Selective cytotoxicity by BKAc-Puro on prostate cancer cells. Prostate cancer (PC-3, DU-145, and LNCaP), colon cancer (HCT116 as a positive control sensitive to the drug), and normal colon (CCD841-CoN as a negative control) cells were grown and analyzed as in FIG. 13. BKAc-Puro can effectively cause cell death on prostate cancer cells.

FIG. 20. In vivo anticancer efficacy of BKAc-Puro. (a) Inhibition of tumor growth in mouse xenograft model. HCT116 cells were implanted subcutaneously into female nude mice. When the developed tumors reached 200 mm³, animals were intraperitoneally injected by the indicated dose of the compound or vehicle control (DMSO). Tumor volume was estimated by the equation vol=(a×b²)/2, where vol, a, and b represent volume, the length of the major axis, and the length of the minor axis, respectively. Arrows indicate the time points of treatment. (b) Body weight change during the treatment in a. Value is expressed in percent change compared to the day of the first treatment. In vivo anticancer efficacy of BKAc-Puro was demonstrated by the inhibition of tumor growth in mouse xenograft model. No toxicity was observed in the group treated with 400 mg/kg of the drug during and after 14 times injections.

FIG. 21. HCT116 cells were implanted subcutaneously into female nude mice. When palpable tumors reached 50-150 mm³, animals were randomized and treated by the indicated dose of the compound or vehicle control (DMSO) via intraperitoneal injection. Tumor incidence is presented as number of mice developed tumor (volume >300 mm³) after 2 weeks of daily treatment/number of mice treated. Tumor volume was estimated by the equation vol=(a×b²)/2, where vol, a, and b represent volume, the length of the major axis, and the length of the minor axis, respectively. In vivo anticancer efficacy of BKAc-Puro was demonstrated by the inhibition of tumor incidence in mouse xenograft model. Consistent with the data presented above, no toxicity was observed in the groups treated with the drug.

FIG. 22. Dose escalation toxicity study. Female nude mice (8-10 weeks old) were treated with the indicated dose of BKAc-Puro (7, 20, 67, 200, and 400 mg/kg; n=3 in each treatment group). Body weight change during the treatment is expressed in percent change compared to the day of the first treatment. Arrows indicate the time points of treatment. Data represent mean values±s.d. (n=3). No severe systemic toxicity was observed up to 400 mg/kg from 5 consecutive daily injections. Considering that the LD50 values for single administration of unmasked puromycin are 335 mg/kg (intravenously), 580 mg/kg (intraperitoneally), and 720 mg/kg (orally) [ABANAE Antibiotics Annual, 1954/1955], BKAc-Puro appears to be well-protected and well-tolerated in animals.

FIG. 23. A. Synthesis of Boc-Lys(Ac)-5-fluorocytidine (BKAc-5FCR). B. Mechanism of prodrug activation by HDAC and tumor-associated protease.

FIG. 24. Effect of the prodrug on cell viability of BxPC3 cells (shGFP and shSki). Cells were treated with the indicated dose of drugs (BKAc-5FCR or 5FCR) for 72 hr followed by MTT assay. Data represent mean values of triplicate measurements±standard deviation. IC₅₀ values were derived from nonlinear curve fit of the dose response data using an outlier's exclusion, variable slope model (GraphPad Prism software). All values are means of at least two independent experiments.

FIG. 25. Selective cytotoxicity by BKAc-5FCR. Human pancreatic BXPC3 cells were treated with DMSO (vehicle), BKAc-5FCR (20 μM), or 5FCR (20 μM) for 72 hr with or without TSA (50 nM) followed by MTS cell viability assay. Representative phase contrast images of cells treated with DMSO (vehicle) or BKAc-5FCR (20 μM) for 72 hr with or without TSA (50 nM) are shown. The HDAC-dependent activation of BKAc-5FCR in BXPC-3 cells was further demonstrated in the presence of HDAC inhibitor TSA where the cytotoxic effect of BKAc-5FCR was substantially compromised.

FIG. 26. Nucleoside antimetabolite analogs.

FIG. 27. Levels of Puro-incorporated proteins in the cells were monitored by immunoblotting. Indicated cell lines were treated either with vehicle control DMSO or Boc-KAc-Puro (16.9 μM) in the presence or absence of TSA (0.5 μM) for 20 h, followed by preparation of cell lysates. The lower panel (anti-α-Tubulin) serves as a loading control.

FIG. 28. Levels of Puro-incorporated proteins in the cells were monitored by immunoblotting. Indicated cell lines were treated either with vehicle control DMSO or Puro (2.1 μM) in the presence or absence of TSA (0.5 μM) for 16 h, followed by preparation of cell lysates. The middle panel shows lighter exposure of the same blot in the top panel. The lower panel (anti-α-Tubulin) serves as a loading control.

FIG. 29. In vivo anticancer efficacy of Boc-KAc-Puro. (a) Tumor growth in HCT116 xenograft model. Animals were treated with acidified saline control or Boc-KAc-Puro (50 or 150 mg/kg). Values are mean±s.d. (n=5 mice per group). *** P<0.001 compared to the control group. P=0.000716 (50 mg/kg), P=0.000349 (150 mg/kg) at 10 d. (b) Body weight change during the treatment in a and was monitored as a sign of general animal health. Values are mean±s.d. (c) Tumor growth in HT29 xenograft model. Animals were treated with acidified saline control or Boc-KAc-Puro (150 mg/kg). Values are mean±s.d. (n=5 mice per group). *** P<0.001 compared to the control group. P=0.000514 (150 mg/kg) at 10 d. (d) Body weight change during the treatment in c was monitored as a sign of general animal health. Values are mean±s.d. (e) Levels of Puro-incorporated proteins were monitored by immunoblotting. Animals bearing HT29 xenograft tumor were given daily intraperitoneal administration of the prodrug at 150 mg/kg/dose for 3 d, followed by preparation of tissue lysates. The lower panel (anti-α-Tubulin) served as a loading control.

FIG. 30. Structures of masked puromycin imaging probes.

FIG. 31. Live single cell imaging by detecting cell surface proteins labeled with Puro using fluorescence microscopy.

FIG. 32. Live single cell imaging by detecting cell surface proteins labeled with Puro using FACS.

### DETAILED DESCRIPTION OF THE INVENTION

The present invention provides a compound having the structure:

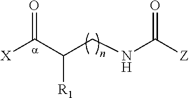

wherein
X is a therapeutic agent containing at least one amine nitrogen and the amine nitrogen on the therapeutic agent covalently bonds directly to carbon α;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

In some embodiments, the compound having the structure:

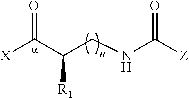

wherein
X is a therapeutic agent containing at least one amine nitrogen and the amine nitrogen on the therapeutic agent covalently bonds directly to carbon α;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

In some embodiments, the compound having the structure:

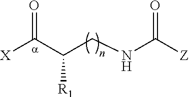

wherein
X is a therapeutic agent containing at least one amine nitrogen and the amine nitrogen on the therapeutic agent covalently bonds directly to carbon α;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
   wherein an amine of the amino acid or oligopeptide is substituted or unsubstituted; and
n is an integer from 0 to 6;
   or a diastereomer, enantiomer or pharmaceutically acceptable salt of the compound.

In some embodiments, the compound wherein
R₁ is —NR₂R₃, —NH—C(═O)—R₄, —NH—C(═O)—OR₄,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkyiheteroaryl, an amino acid or oligopeptide;
      wherein an amine of the amino acid or oligopeptide is substituted or unsubstituted; and
   n is 4.

In some embodiments, the compound wherein n is 3, 4 or 5.

In some embodiments, the compound wherein
R₁ is —NR₂R₃,
   wherein
   R₂ is —H; and
   R₃ is an amino acid,
      wherein the amino acid is bonded to the nitrogen through an amide bond.

In some embodiments, the compound wherein
R₁ is —NR₂R₃,
   wherein
   R₂ is —H; and
   R₃ is an oligopeptide,
      wherein the oligopeptide is bonded to the nitrogen through an amide bond.

In some embodiments, the compound wherein
R₁ is

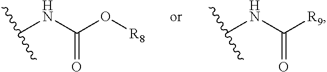

wherein R₈ and R₉ are each independently —H, —CH₃, t-butyl, phenyl, or benzyl.

In some embodiments, the compound wherein
R₁ is

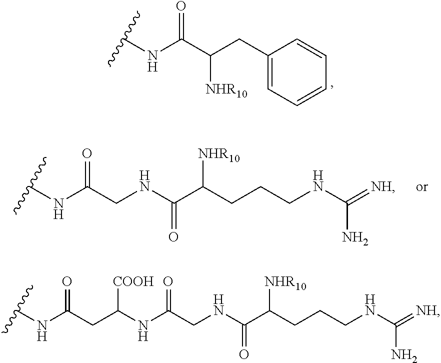

wherein R₁₀ is —H, —CH₃, Ac, —C(O)—Ot-Bu, —C(O)—OCH₂Ph, —CHO, phenyl, or benzyl.

In some embodiments, the compound wherein X is a chemotherapeutic agent containing at least one amine nitrogen.

In some embodiments, the compound wherein X is a nucleoside or deoxynucleoside containing at least one amine nitrogen.

In some embodiments, the compound wherein X is puromycin, 5-fluorocytidine, 2′-deoxy-5-fluorocytidine, 5′-deoxy-5-fluorocytidine, 5′-deoxy-5-fluorocytidine, gemcitabine, cytarabine, cladribine, troxacitabine, adriamycin, alimta, aminolevulinic acid, azacitidine, bleomycin, cerubidine, clofarabine, clofarex, crizotinib, dasatinib, daunorubicin, decitabine, doxil, deoxorubicin, ellence, epirubicin, eribulin mesylate, erlotinib, evacet, fludara, fludarabine phosphate, fluorouracil, fulvestrant, gefitinib, gemcitabine hydrochloride, imiquimod, lapatinib ditosylate, lenalidomide, letrozole, leucovorin, levulan, matulane, methotrexate, mitomycin C, nelarabine, nilotinib, pazopanib, pemetrexed, pralatrexate, prednisone, wellcovorin, xalkori, discodermolide, or blasticidin.

The present invention provides compound having the structure:

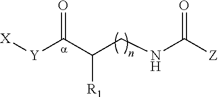

wherein
X is a therapeutic agent;
Y is a chemical linker;
   wherein Y is present or absent, and when present,
   Y is a chemical linker containing at least one amine nitrogen,
      wherein the amine nitrogen on the linker covalently bonds directly to carbon α, or
   Y is a para-aminobenzyl alcohol linker,
      wherein the nitrogen on the linker Y connects directly to carbon α and the oxygen on the linker Y connects to the therapeutic agent X, or the oxygen on the linker Y connects directly to carbon α and the nitrogen on the linker Y connects to the therapeutic agent X through an amide bond;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein the amine of the amino acid or oligopeptide is substituted or unsubstituted; and
n is an integer from 0 to 6;
   or a diastereomer, enantiomer or pharmaceutically acceptable salt of the compound.

In some embodiment, the compound having the structure:

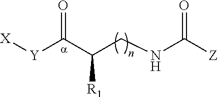

wherein
X is a therapeutic agent;
Y is a chemical linker;
   wherein Y is present or absent, and when present, Y is a chemical linker containing at least one amine nitrogen,
      wherein the amine nitrogen on the linker covalently bonds directly to carbon α, or
   Y is a para-aminobenzyl alcohol linker,
      wherein the nitrogen on the linker Y connects directly to carbon α and the oxygen on the linker Y connects to the therapeutic agent X, or the oxygen on the linker Y connects directly to carbon α and the nitrogen on the linker Y connects to the therapeutic agent X through an amide bond;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein the amine of the amino acid or oligopeptide is substituted or unsubstituted; and
n is an integer from 0 to 6;
   or a diastereomer, enantiomer or pharmaceutically acceptable salt of the compound.

In some embodiments the compound having the structure:

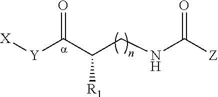

wherein
X is a therapeutic agent;
Y is a chemical linker;
   wherein Y is present or absent, and when present, Y is a chemical linker containing at least one amine nitrogen,
      wherein the amine nitrogen on the linker covalently bonds directly to carbon α, or
   Y is a para-aminobenzyl alcohol linker,
      wherein the nitrogen on the linker Y connects directly to carbon α and the oxygen on the linker Y connects to the therapeutic agent X, or the oxygen on the linker Y connects directly to carbon α and the nitrogen on the linker Y connects to the therapeutic agent X through an amide bond;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein the amine of the amino acid or oligopeptide is substituted or unsubstituted; and
n is an integer from 0 to 6;
   or a diastereomer, enantiomer or pharmaceutically acceptable salt of the compound.

In some embodiments, the compound wherein
R₁ is —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein the amine of the amino acid or oligopeptide is substituted or unsubstituted; and
n is 4.

In some embodiments, the compound wherein
Y is present, and Y is a chemical linker containing at least one amine nitrogen,
   wherein the amine nitrogen on the linker covalently bonds directly to carbon α.

In some embodiments, the compound wherein
Y is present, and Y is a para-aminobenzyl alcohol linker, wherein the nitrogen on the linker Y connects to directly to carbon α and the oxygen on the linker Y connects to the therapeutic agent X, or the oxygen on the linker Y connects directly to carbon α and the nitrogen on the linker Y connects to the therapeutic agent X through an amide bond.

In some embodiments, the compound wherein the nitrogen on the linker Y connects to directly to carbon α and the oxygen on the linker Y connects to the therapeutic agent X.

In some embodiments, the compound wherein the oxygen on the linker Y connects directly to carbon α and the nitrogen on the linker Y connects to the therapeutic agent X through an amide bond.

In some embodiments, the compound wherein n is 3, 4 or 5.

In some embodiments, the compound wherein
Y is absent.

In some embodiments, the compound wherein
Y is present.

In some embodiments, the compound wherein
Y is present and
Y is

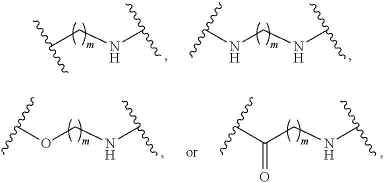

wherein
m is 0 to 6; and
a nitrogen on the linker Y connects to directly to carbon α.

In some embodiments, the compound wherein
R₁ is —NR₂R₃,
   wherein
   R₂ is —H; and
   R₃ is an amino acid,
      wherein the amino acid is bonded to the nitrogen through an amide bond.

In some embodiments, the compound wherein
R₁ is —NR₂R₃,
   wherein
   R₂ is —H; and
   R₃ is an oligopeptide,
      wherein the oligopeptide is bonded to the nitrogen through an amide bond.

In some embodiments, the compound wherein
R₁ is

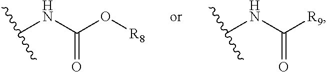

wherein R₈ and R₉ are each independently —H, —CH₃, t-butyl, phenyl, or benzyl.

In some embodiments, the compound wherein
R₁ is

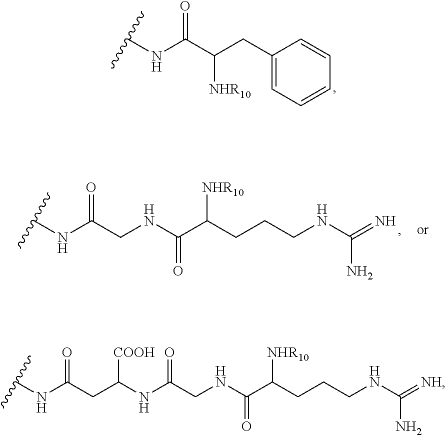

wherein R₁₀ is —H, —CH₃, Ac, —C(O)—Ot-Bu, —C(O)—OCH₂Ph, —CHO, phenyl, or benzyl.

In some embodiments, the compound wherein Y is absent, and the therapeutic agent X contains at least one amine and an amine nitrogen on the therapeutic agent X connects directly to carbon α.

In some embodiments, the compound wherein X is a chemotherapeutic agent.

In some embodiments, the compound wherein X is a nucleoside or deoxynucleoside.

In some embodiments, the compound wherein X is puromycin, 5-fluorocytidine, 2′-deoxy-5-fluorocytidine, 5′-deoxy-5-fluorocytidine, 5′-deoxy-5-fluorocytidine, gemcitabine, cytarabine, cladribine, troxacitabine, abiraterone acetate, abraxane, adriamycin, afinitor, alimta, aloxi, amboclorin, aminolevulinic acid, anastrozole, aprepitant, aromasin, axitinib, azacitidine, bendamustine hydrochloride, bexarotene, bleomycin, bortezomib, cabazitaxel, capecitabine, cerubidine, clofarabine, clofarex, crizotinib, dacarbazine, dasatinib, daunorubicin hydrochloride, decitabine, degarelix, dexrazoxane hydrochloride, docetaxel, doxil, deoxorubicin, ellence, epirubicin hydrochloride, eribulin mesylate, erlotinib hydrochloride, etoposide, evacet, everolimus, fludara, fludarabine phosphate, fluorouracil, fulvestrant, gefitinib, gemcitabine hydrochloride, imatinib mesylate, imiquimod, irinotecan hydrochloride, ixabepilone, lapatinib ditosylate, lenalidomide, letrozole, leucovorin calcium, leuprorelin, levulan, lomustine, lupron, matulane, methotrexate, mitomycin C, navelbine, nelarabine, nexavar, nilotinib, nolvadex, palonosetron hydrochloride, pazopanib hydrochloride, pemetrexed disodium, pralatrexate, prednisone, procarbazine hydrochloride, raloxifene hydrochloride, ruxolitinib phosphate, sorafenib tosylate, sunitinib malate, tamoxifen citrate, taxol, taxotere, temozolomide, temsirolimus, topotecan hydrochloride, toremifene, vandetanib, vemurafenib, vinblastine sulfate, vincristine sulfate, vinorelbine tartrate, vismodegib, wellcovorin, xalkori, zevalin, zinecard, zoledronic acid, discodermolide, or blasticidin.

In some embodiments, the compound wherein Z is CH₃.

In some embodiments, the compound wherein Z is CF₃.

In some embodiments, the compound
wherein R₁ is

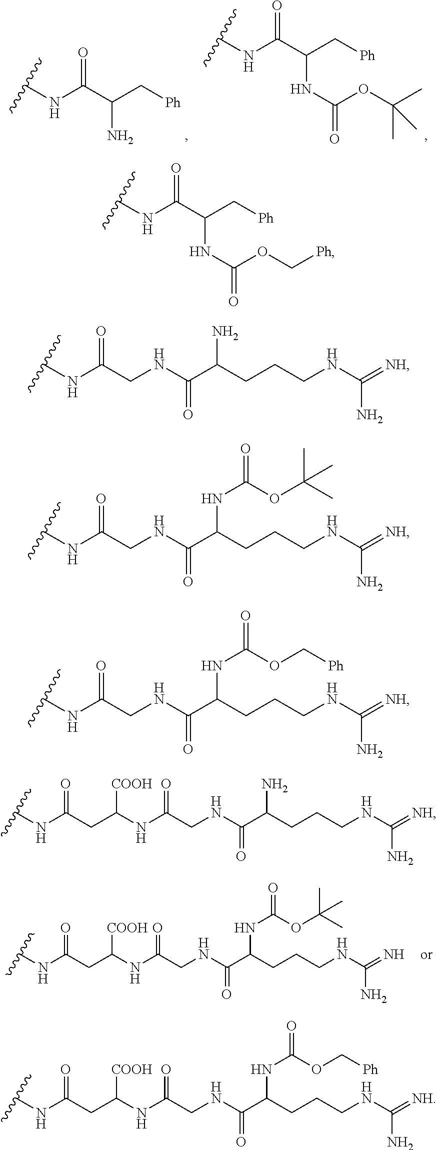

In some embodiments, the compound having the structure:

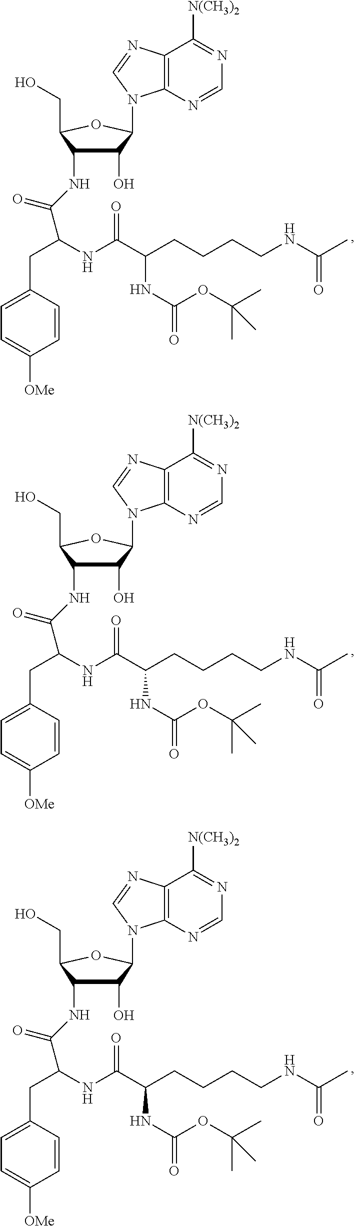

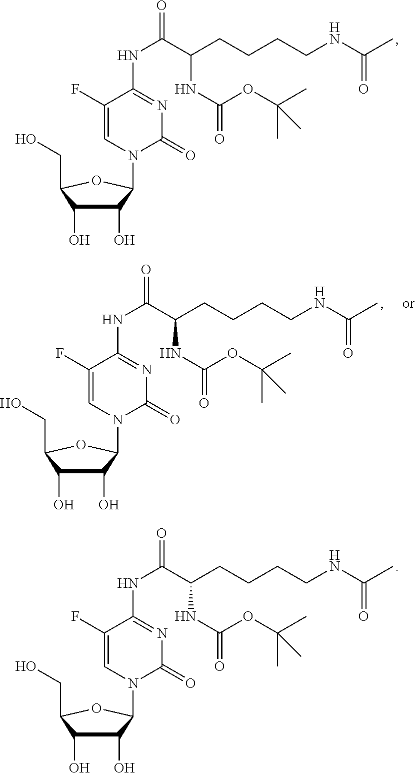

In some embodiments, the compound having the structure:

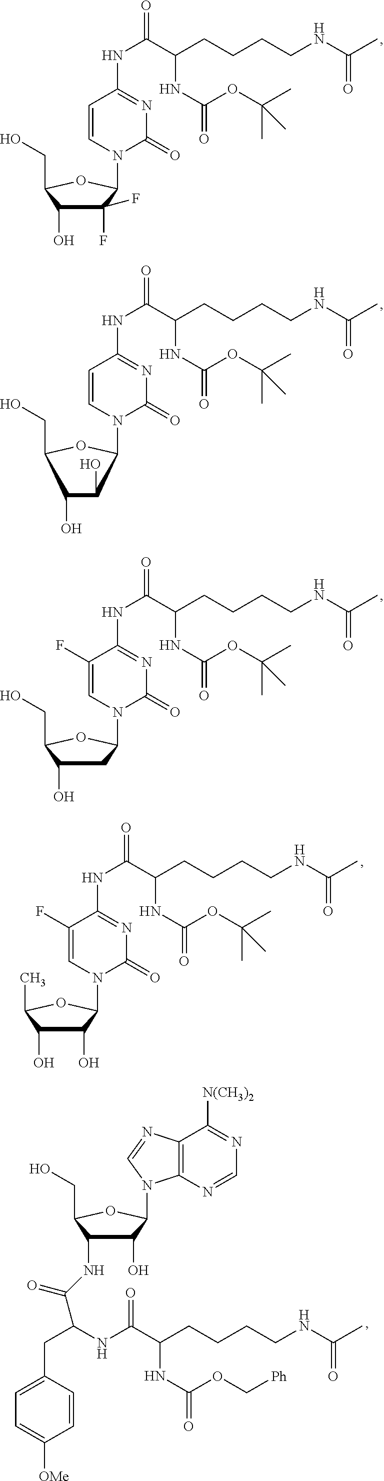

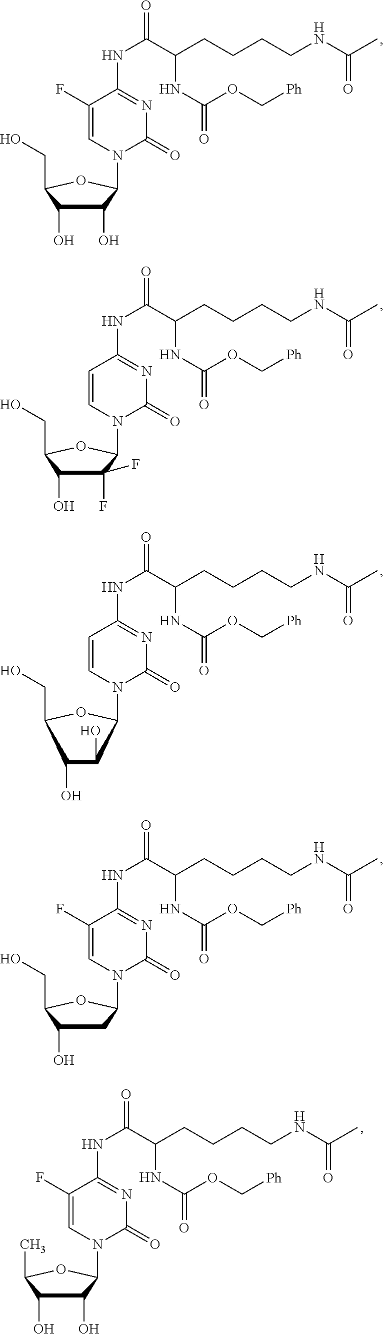

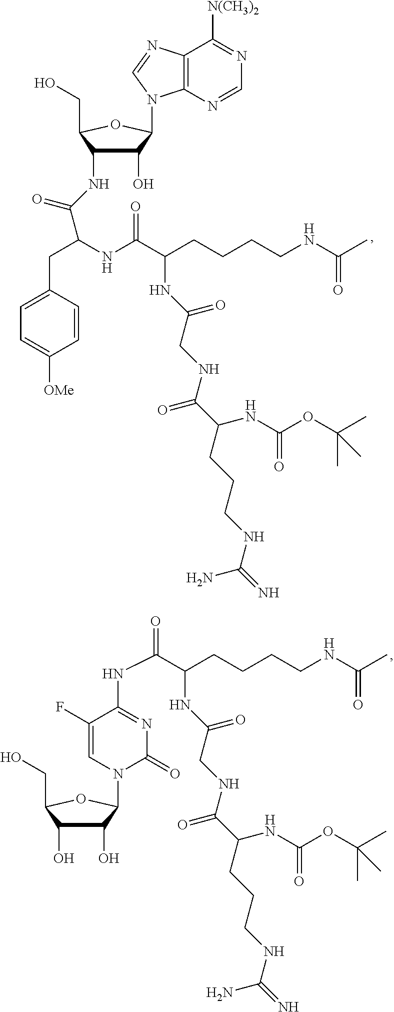

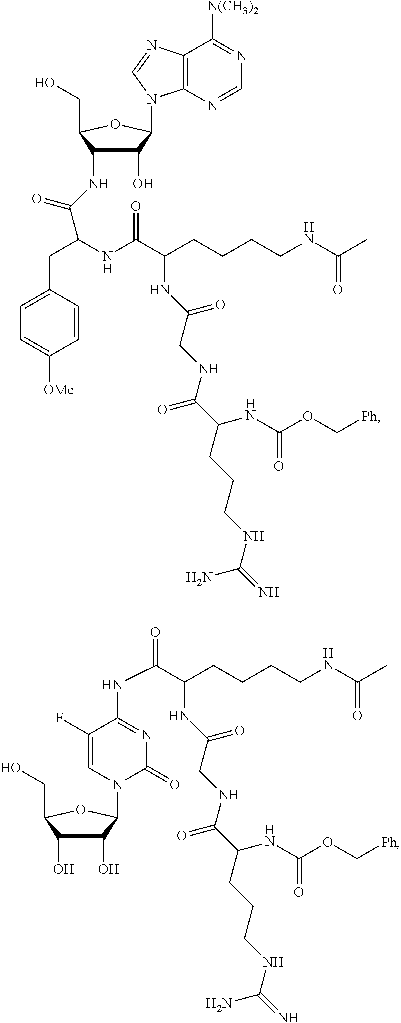

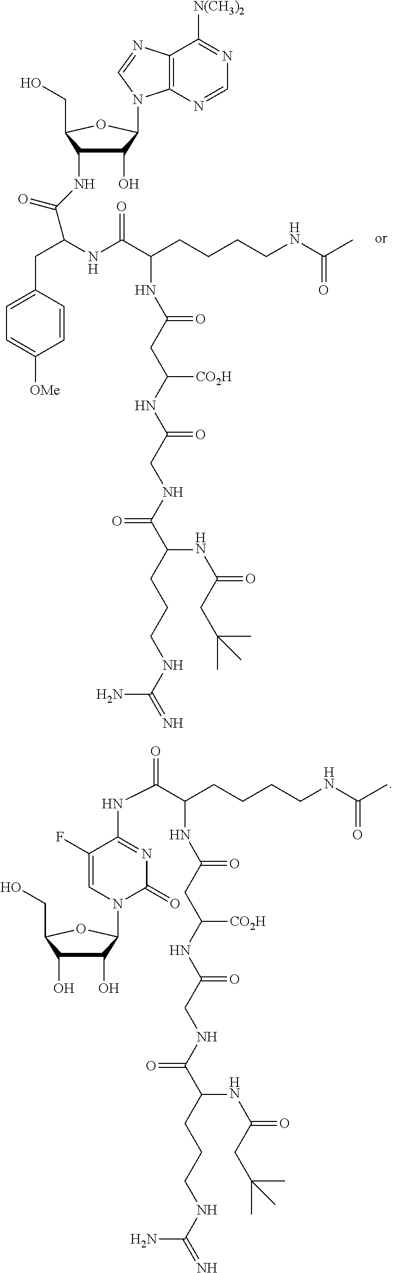

In some embodiments, the compound wherein
R₁ is —NHBoc;
n is 4;
X is puromycin or 5-fluorocytidine; and
Z is CH₃.

In some embodiments, a pharmaceutical composition comprising the compound of the present invention and a pharmaceutically acceptable carrier.

In some embodiments, a method for reducing one or more symptoms of disease in a subject, comprising administering an effective amount of the compound of the present invention or the composition of the present invention to the subject so as to treat the disease in the subject.

In some embodiments, the disease is characterized by or caused by cells which have elevated levels of histone deacetylases or proteases or both.

In some embodiments, the disease is cancer.

In some embodiments, the compound or composition inhibits cancer cell metastasis.

In some embodiments, the compound or composition inhibits cancer cell proliferation.

In some embodiments, the cancer cells have elevated levels of histone deacetylases or proteases or both.

In some embodiments, the cancer is colon, pancreatic, liver, breast, prostate, or cervical cancer.

In some embodiments, a method for inhibiting growth of a tumor comprising contacting the tumor with a compound of the present invention or the composition of the present invention.

In some embodiments, a method for reducing the size a tumor comprising contacting the tumor with a compound of the present invention or the composition of the present invention.

In some embodiments, the compound or composition of the present invention for use in treating a subject suffering from cancer.

In some embodiments, the compound or composition of the present invention for use in treating cancer.

In some embodiments, the compound or composition of the present invention for use in inhibiting growth of a tumor.

In some embodiments, the compound or composition of the present invention for use in reducing the size a tumor.

In some embodiments, the compound or composition of the present invention for use in treating a disease that is caused by cells which have elevated levels of histone deacetylases or proteases or both.

The present invention provides a compound having the structure:

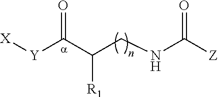

wherein
X is an imaging agent containing at least one amine nitrogen;
Y is a chemical linker,
   wherein Y is present or absent, and when present Y is a chemical linker containing at least one amine nitrogen or Y is a para-aminobenzyl alcohol linker;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

The present invention provides a compound having the structure:

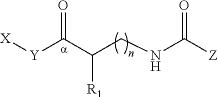

wherein
X is an imaging agent containing at least one amine nitrogen;
Y is a chemical linker,
   wherein Y is present or absent, and when present Y is a chemical linker containing at least one amine nitrogen or Y is a para-aminobenzyl alcohol linker;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
wherein
when Y is absent, an amine nitrogen on the imaging agent covalently bonds directly to carbon α, or
when Y is present and is a chemical linker containing at least one amine nitrogen, an amine nitrogen on the linker covalently bonds directly to carbon α, or
when Y is present and is a para-aminobenzyl alcohol linker,
the nitrogen on the linker Y bonds directly to carbon α and the oxygen on the linker Y connects to the imaging agent X, or the oxygen on the linker Y bonds directly to carbon α and the nitrogen on the linker Y connects to the imaging agent X through an amide bond;
   or a diastereomer, enantiomer or pharmaceutically acceptable salt of the compound.

In some embodiments, the compound having the structure

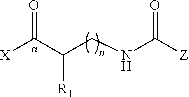

wherein
X is an imaging agent containing at least one amine nitrogen and the amine nitrogen on the imaging agent covalently bonds directly to carbon α;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

In some embodiments, the compound having the structure:

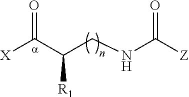

wherein
X is an imaging agent containing at least one amine nitrogen and the amine nitrogen on the imaging agent covalently bonds directly to carbon α;
Z is CH₃ or CF₃;
R₁ is —H, —NR₃R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

In some embodiments, the compound having the structure:

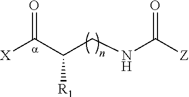

wherein
X is a an imaging agent containing at least one amine nitrogen and the amine nitrogen on the imaging agent covalently bonds directly to carbon α;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

In some embodiments, the compound,
wherein
R₁ is —NR₂R₃, —NH—C(═O)—R₄, —NH—C(═O)—OR₄,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or oligopeptide;
   wherein an amine of the amino acid or oligopeptide is substituted or unsubstituted; and
n is 4.

In some embodiments, the compound having the structure:

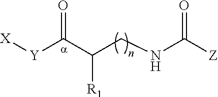

wherein
X is an imaging agent;
Y is a chemical linker;
   wherein Y is present or absent, and when present,
   Y is a chemical linker containing at least one amine nitrogen,
      wherein the amine nitrogen on the linker covalently bonds directly to carbon α, or
   Y is a para-aminobenzyl alcohol linker,
      wherein the nitrogen on the linker Y connects directly to carbon α and the oxygen on the linker Y connects to the imaging agent X, or the oxygen on the linker Y connects directly to carbon α and the nitrogen on the linker Y connects to the imaging agent X through an amide bond;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein the amine of the amino acid or oligopeptide is substituted or unsubstituted; and
n is an integer from 0 to 6;
   or a diastereomer, enantiomer or pharmaceutically acceptable salt of the compound.

In some embodiments, the compound having the structure:

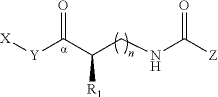

wherein
X is an imaging agent;
Y is a chemical linker;
   wherein Y is present or absent, and when present,
   Y is a chemical linker containing at least one amine nitrogen,
      wherein the amine nitrogen on the linker covalently bonds directly to carbon α, or
   Y is a para-aminobenzyl alcohol linker,
      wherein the nitrogen on the linker Y connects directly to carbon α and the oxygen on the linker Y connects to the imaging agent X, or the oxygen on the linker Y connects directly to carbon α and the nitrogen on the linker Y connects to the imaging agent X through an amide bond;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein the amine of the amino acid or oligopeptide is substituted or unsubstituted; and
n is an integer from 0 to 6;
   or a diastereomer, enantiomer or pharmaceutically acceptable salt of the compound.

In some embodiments, the compound having the structure:

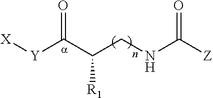

wherein
X is an imaging agent;
Y is a chemical linker;
   wherein Y is present or absent, and when present,
   Y is a chemical linker containing at least one amine nitrogen,
      wherein the amine nitrogen on the linker covalently bonds directly to carbon α, or
   Y is a para-aminobenzyl alcohol linker,
      wherein the nitrogen on the linker Y connects directly to carbon α and the oxygen on the linker Y connects to the imaging agent X, or the oxygen on the linker Y connects directly to carbon α and the nitrogen on the linker Y connects to the imaging agent X through an amide bond;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein the amine of the amino acid or oligopeptide is substituted or unsubstituted; and
n is an integer from 0 to 6;
   or a diastereomer, enantiomer or pharmaceutically acceptable salt of the compound.

In some embodiments, the compound
wherein
R₁ is —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein the amine of the amino acid or oligopeptide is substituted or unsubstituted; and
n is 4.

In some embodiments, the compound
wherein Y is present, and Y is a chemical linker containing at least one amine nitrogen,
   wherein the amine nitrogen on the linker covalently bonds directly to carbon α.

In some embodiments, the compound
wherein Y is present, and Y is a para-aminobenzyl alcohol linker,
   wherein the nitrogen on the linker Y connects to directly to carbon α and the oxygen on the linker Y connects to the imaging agent X, or the oxygen on the linker Y connects directly to carbon α and the nitrogen on the linker Y connects to the imaging agent X through an amide bond.

In some embodiments, the compound
wherein the nitrogen on the linker Y connects to directly to carbon α and the oxygen on the linker Y connects to the imaging agent X.

In some embodiments, the compound
wherein the oxygen on the linker Y connects directly to carbon α and the nitrogen on the linker Y connects to the imaging agent X through an amide bond.

In some embodiments, the compound wherein Y is absent.

In some embodiments, the compound wherein Y is present.

In some embodiments, the compound wherein Y is present and
Y is

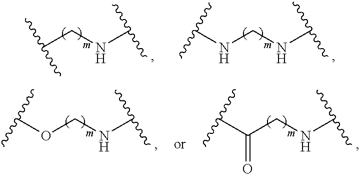

wherein
m is 0 to 6; and
a nitrogen on the linker Y connects to directly to carbon α.

In some embodiments, the compound
wherein Y is absent, and the imaging agent X contains at least one amine and an amine nitrogen on the imaging agent X connects directly to carbon α.

In some embodiments, the compound wherein n is 3, 4 or 5.

In some embodiments, the compound
wherein R₁ is —NR₂R₃,
   wherein
   R₂ is —H; and
   R₃ is an amino acid,
      wherein the amino acid is bonded to the nitrogen through an amide bond.

In some embodiments, the compound
wherein R₁ is —NR₂R₃,
   wherein
   R₂ is —H; and
   R₃ is an oligopeptide,
      wherein the oligopeptide is bonded to the nitrogen through an amide bond.

In some embodiments, the compound
wherein R₁ is

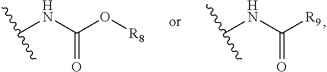

wherein R₈ and R₉ are each independently —H, —CH₃, t-butyl, phenyl, or benzyl.

In some embodiments, the compound
wherein R₁ is

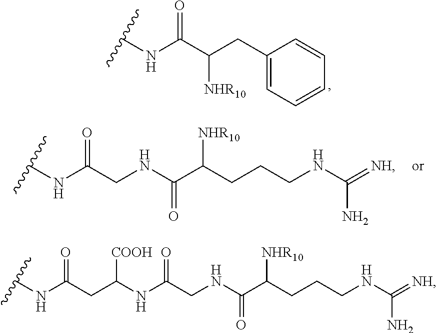

wherein R₁₀ is —H, —CH₃, Ac, —C(O)-Ot-Bu, —C(O)—OCH₂Ph, —CHO, phenyl, or benzyl.

In some embodiments, the compound
wherein R₁ is

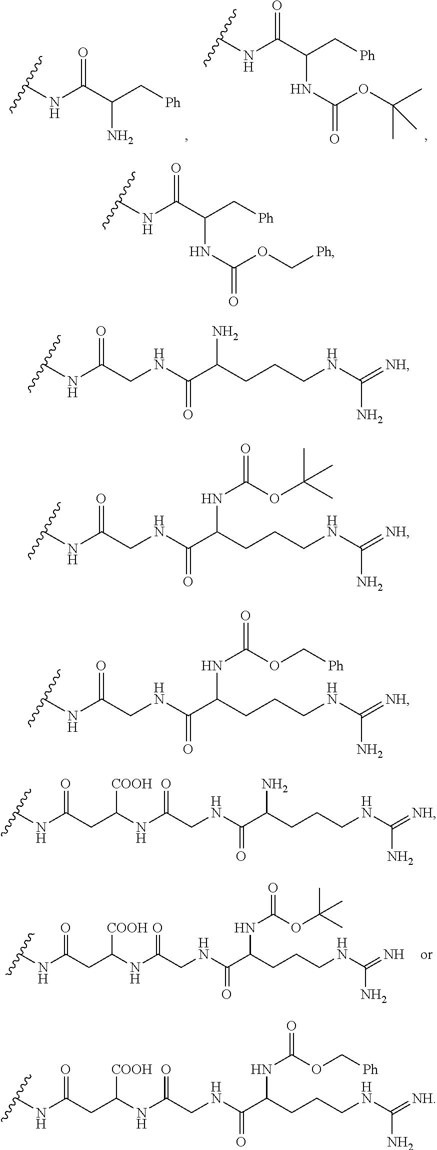

In some embodiments, the compound wherein Z is CH₃.

In some embodiments, the compound wherein Z is CF₃.

In some embodiments, the compound wherein the imaging agent X comprises at least one imaging moiety Q.

In some embodiments, the compound wherein the imaging agent X is puromycin, wherein at least one atom in the puromycin is replaced with a radioisotope of said atom.

In some embodiments, the compound wherein at least one 1H in the puromycin is replaced with ³H, or at least one ¹²C in the puromycin is replaced with ¹¹C, or at least one ¹⁴N in the puromycin is replaced with ¹³N, or at least one ¹⁶O in the puromycin is replaced with ¹⁵O.

In some embodiments, the compound having the structure:

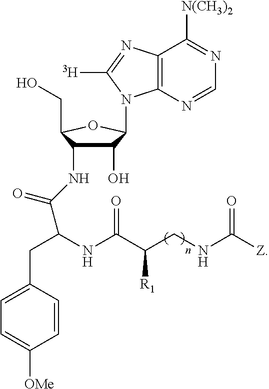

In some embodiments, the compound having the structure:

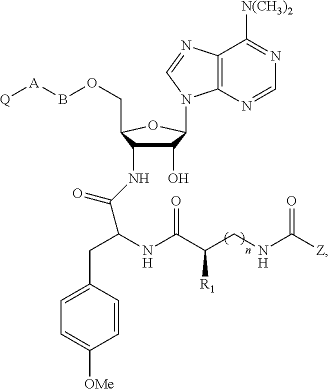

wherein
Q is an imaging moiety;
A is present or absent and when present is a alkyl linker; and
B is present or absent and when present is a nucleoside linker or nucleotide linker.

In some embodiments, the compound having the structure:

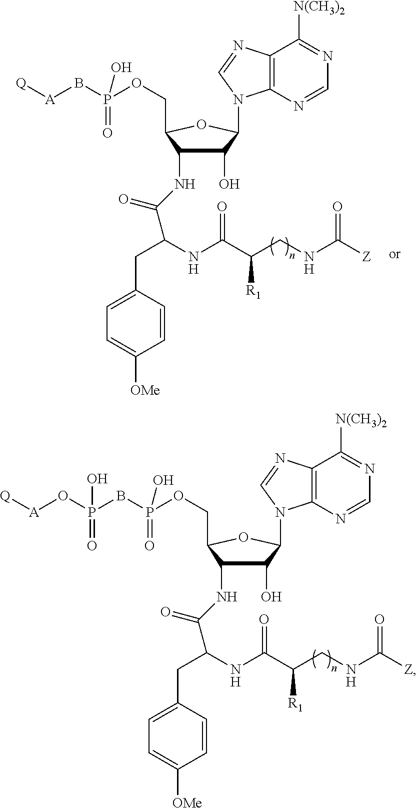

wherein
Q is an imaging moiety;
A is present or absent and when present is a alkyl linker; and
B is present or absent and when present is a nucleoside linker or nucleotide linker.

In some embodiments, the compound
wherein the alkyl linker A is present and the imaging moiety is connected to the alkyl linker by an amide or ester bond.

In some embodiments, the compound having the structure:

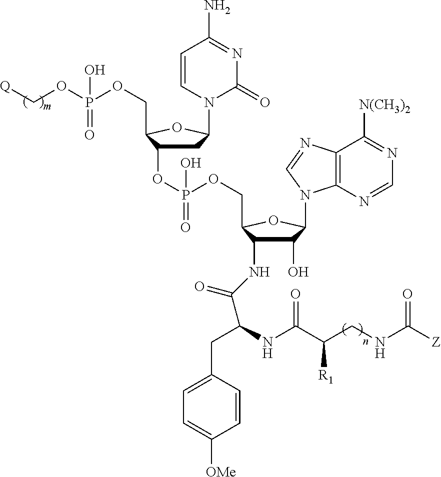

wherein M is an integer from 1 to 12.

In some embodiments, the compound wherein Q is an MRI contrast imaging moiety, an optical imaging moiety, or a PET imaging moiety.

In some embodiments, the compound wherein Q is an MRI contrast imaging moiety.

In some embodiments, the compound wherein Q contains a paramagnetic species.

In some embodiments, the compound wherein the paramagnetic species is Gd³⁺, Fe³⁺, In³⁺, or Mn²⁺.

In some embodiments, the compound wherein Q is a metal chelator.

In some embodiments, the compound wherein the metal chelator is DOTA, NOTA, DTPA, TETA, CB-TE2A or CB-DO2A.

In some embodiments, the compound wherein the metal chelator coordinates to Gd³⁺, Fe³⁺, In³⁺, or Mn²⁺.

In some embodiments, the compound wherein Q is an optical imaging moiety.

In some embodiments, the compound wherein Q contains an imaging dye.

In some embodiments, the compound wherein the imaging dye is a fluorescent dye.

In some embodiments, the compound wherein the fluorescent dye fluoresces in the visible region, the UV region or near-IR region.

In some embodiments, the compound wherein the imaging dye is 6-FAM or Cy5.

In some embodiments, the compound wherein Q contains quantum dots.

In some embodiments, the compound wherein Q is a PET imaging moiety.

In some embodiments, the compound wherein Q contains a radioisotope.

In some embodiments, the compound wherein the radioisotope is ³H, ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁴⁴Sc ⁶⁷Ga, or ⁶⁸Ga.

In some embodiments, the compound wherein Q is a metal chelator.

In some embodiments, the compound wherein the metal chelator is DOTA, NOTA, DTPA, TETA, CB-TE2A or CB-DO2A.

In some embodiments, the compound wherein the metal chelator coordinates to ^{99m}Tc, ⁹⁵Tc, ¹¹¹In, ⁶²Cu, ⁶⁴Cu, ⁴⁴Sc ⁶⁷Ga, or ⁶⁸Ga.

In some embodiments, the compound wherein the metal chelator coordinates to ⁶⁸Ga.

In some embodiments, the compound having the structure:

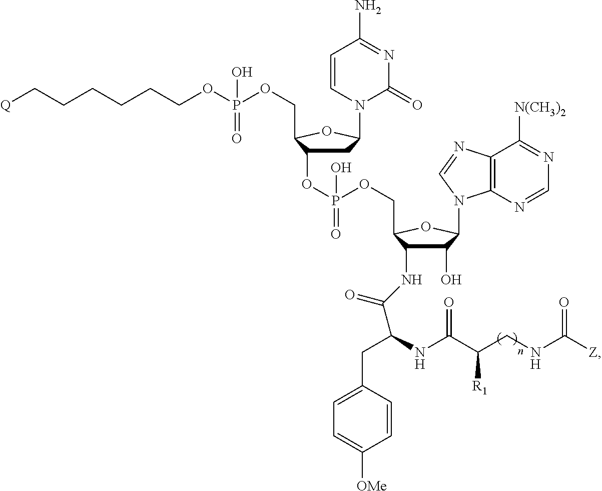

wherein Q is an ester or amide derivative of DOTA, NOTA, DTPA, TETA, CB-TE2A or CB-DO2A which is coordinated to a metal M, wherein the M is Gd³⁺, Fe³⁺, In³⁺, Mn²⁺, ^{99m}TC, ⁹⁵Tc, ¹¹¹In, ⁶²Cu, ⁶⁴Cu, ⁴⁴Sc, ⁶⁷Ga, or ⁶⁸Ga.

In some embodiments, the compound having the structure:

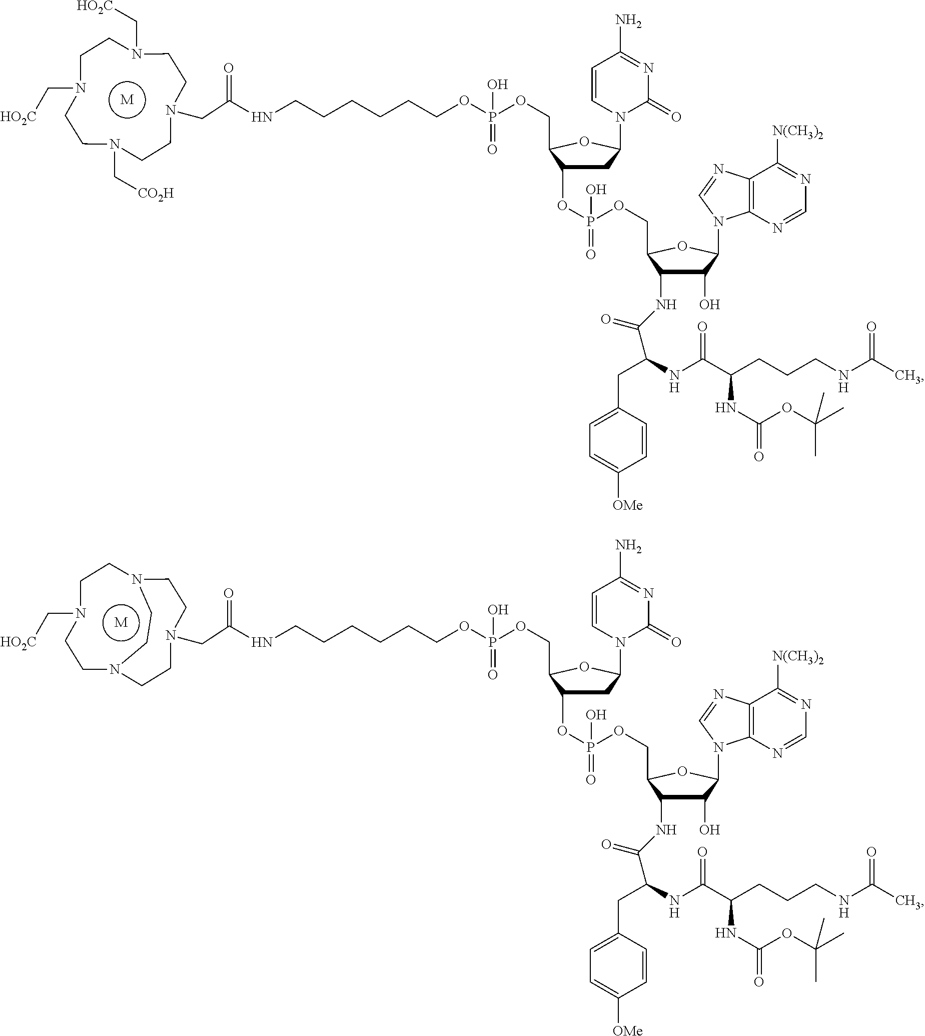

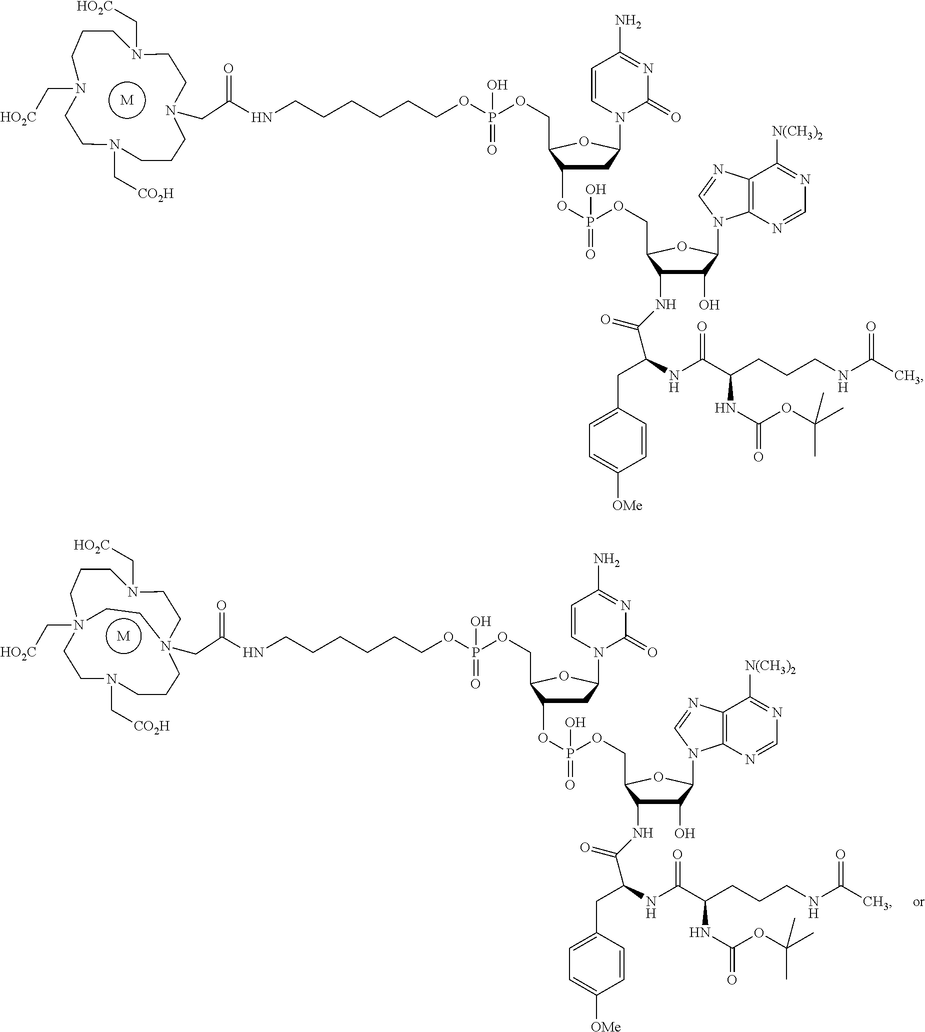

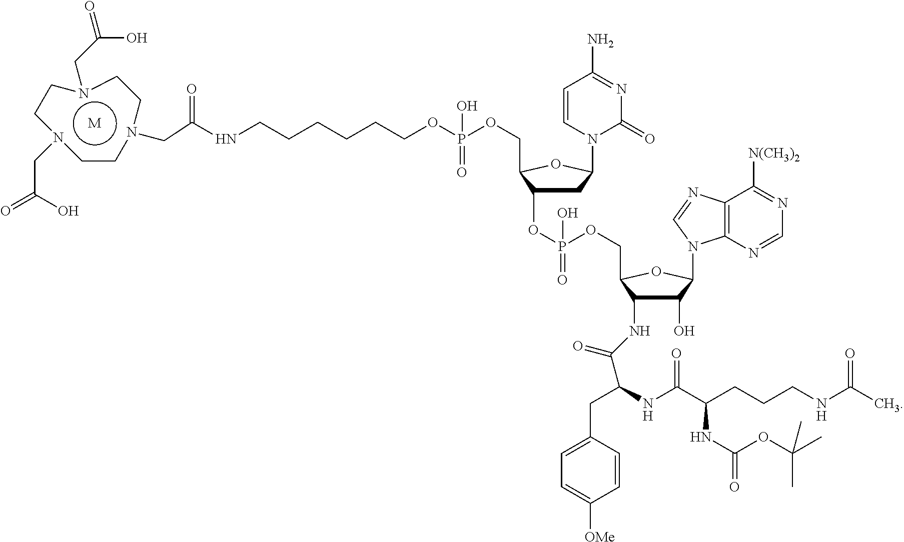

In some embodiments, the compound having the structure:

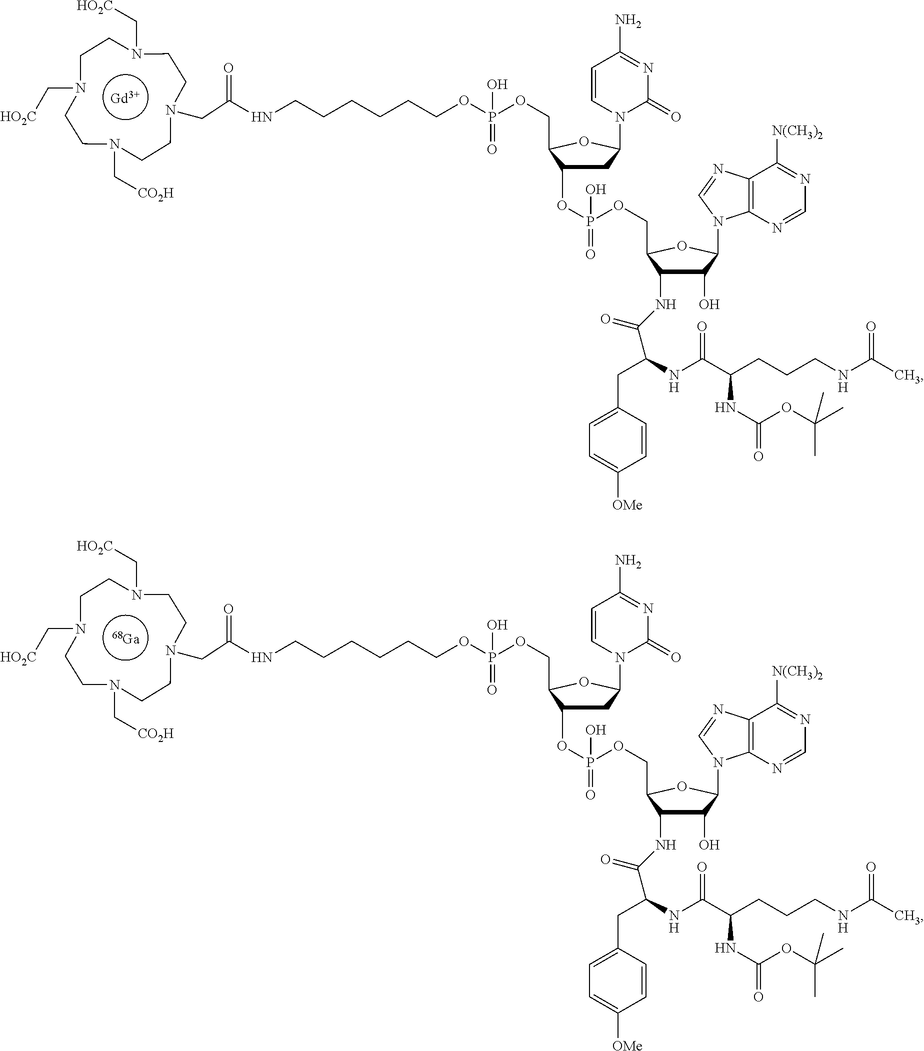

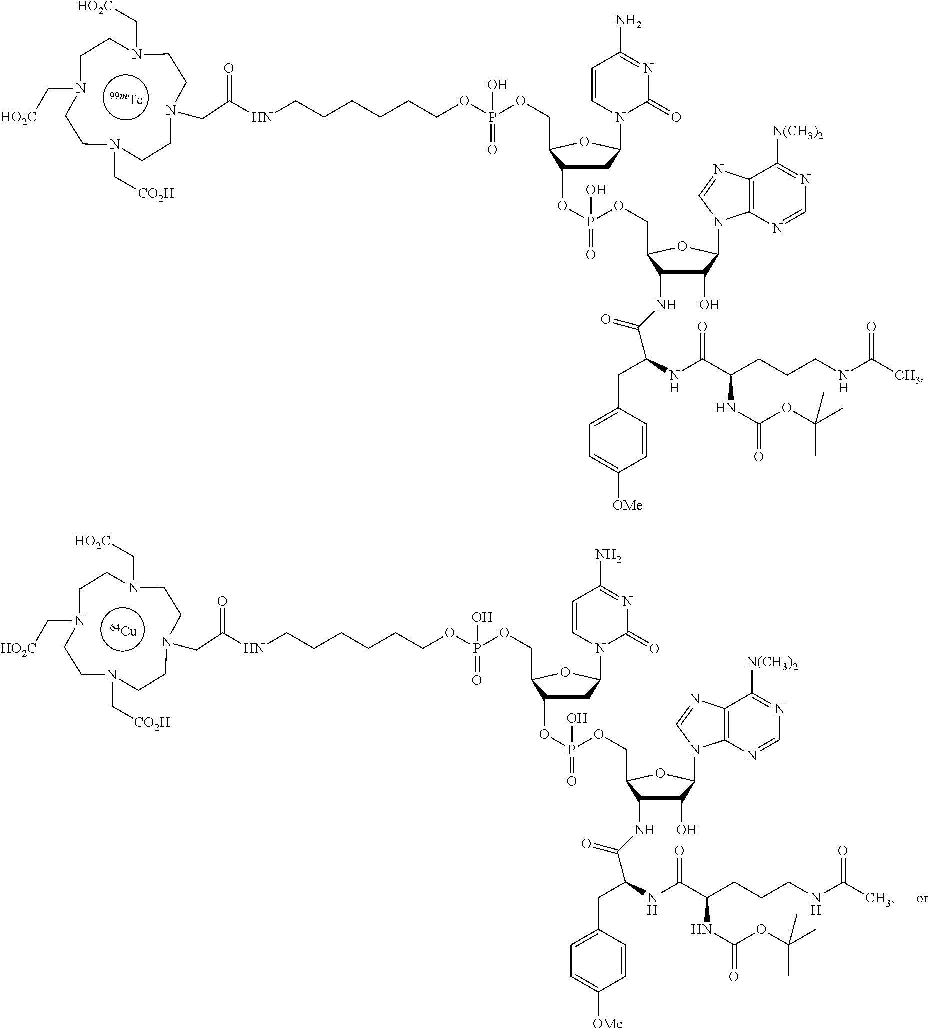

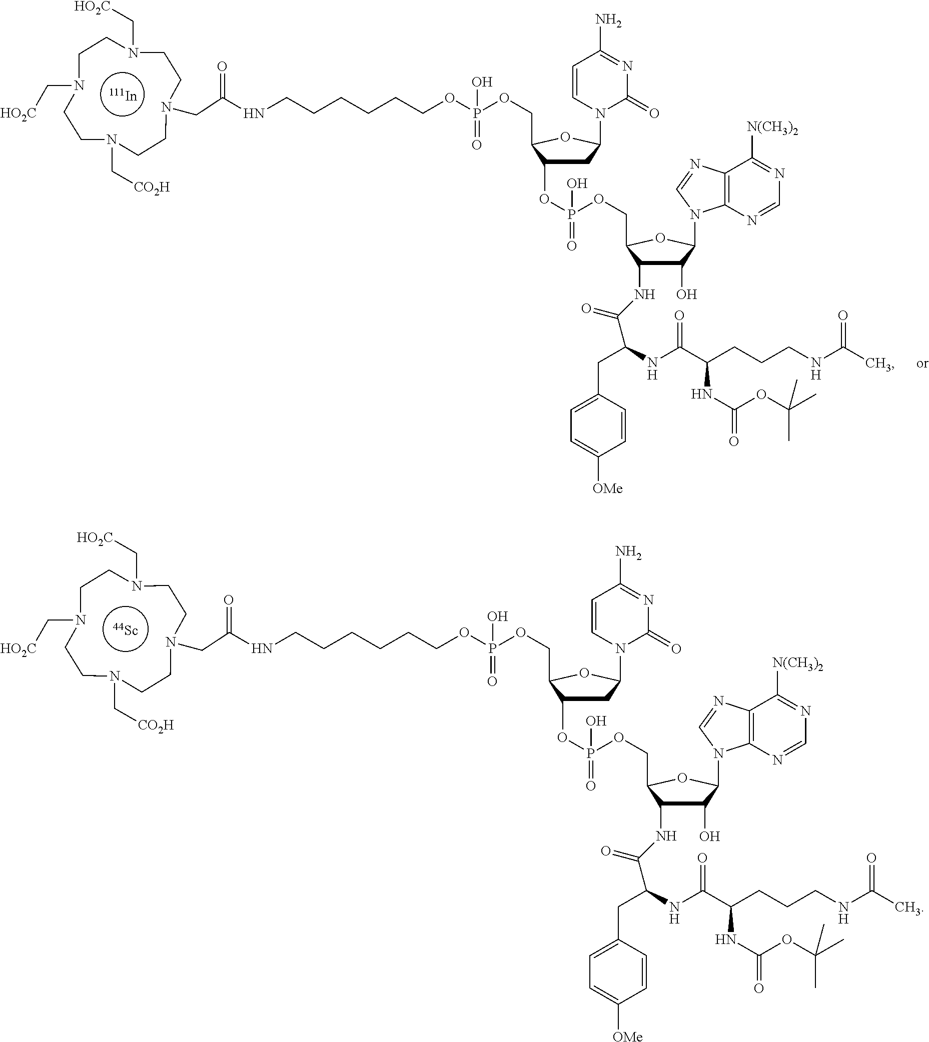

In some embodiments, the compound having the structure

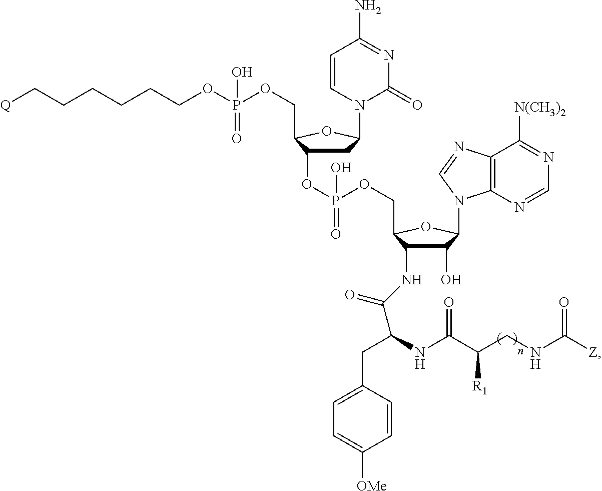

wherein Q is an ester or amide derivative of 6-FAM or Cy5.

In some embodiments, the compound having the structure:

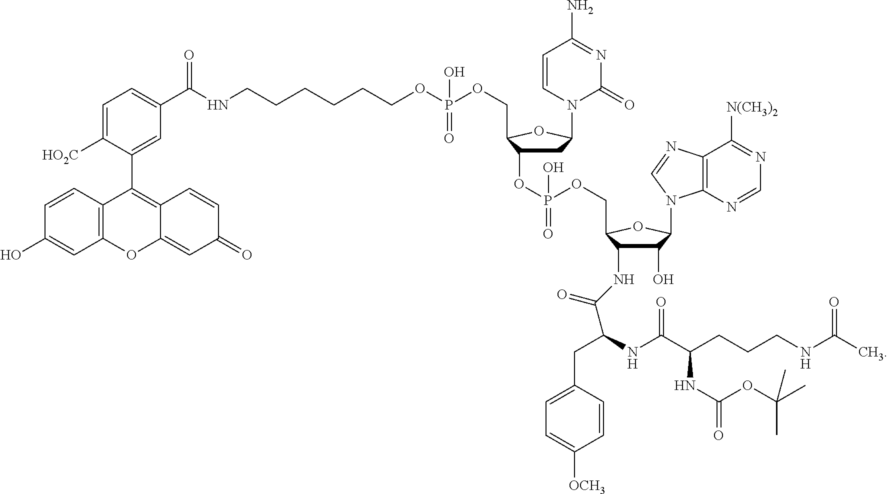

In some embodiments, the compound having the structure:

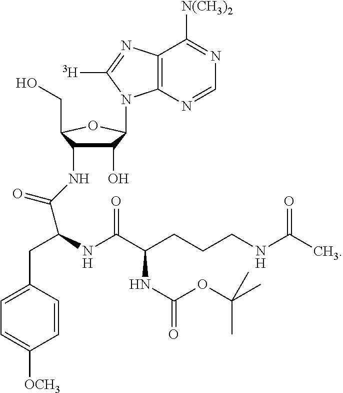

The present invention provides a compound having the structure:

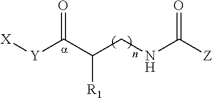

wherein
X is an imaging agent containing at least one amine nitrogen;
Y is a chemical linker,
   wherein Y is present or absent, and when present Y is a chemical linker containing at least one amine nitrogen or Y is a para-aminobenzyl alcohol linker;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

The present invention provides a pharmaceutical composition comprising a compound of the present invention and a pharmaceutically acceptable carrier.

The present invention provides a method for detecting cancer cells in a subject comprising administering an effective amount of a compound of the present invention or a composition of the present invention to the subject, and imaging the subject with a molecular imaging device to detect the compound or composition in the subject.

In some embodiments of the method, wherein the compound or composition specifically accumulates in cancer cells relative to non-cancer cells.

In some embodiments of the method, wherein detection of the compound or composition in an organ of the subject is an indication that cancers cells are present in the organ.

In some embodiments of the method, wherein the cancer cells are lung cancer, breast cancer, prostate cancer, cervical cancer, pancreatic cancer, colon cancer, ovarian cancer, stomach cancer, esophagus cancer, skin cancer, heart cancer, liver cancer, bronchial cancer, testicular cancer, kidney cancer, bladder cancer, spleen cancer, thymus cancer, thyroid cancer, brain cancer, or gall bladder cancer cells.

In some embodiments of the method, wherein the cancer cells have elevated levels of histone deacetylases or proteases or both.

In some embodiments of the method, wherein the compound or composition is detected using an MRI imaging device, a PET imaging device or an optical imaging device.

In some embodiments, the imaging moiety comprises a metal chelator selected from ethylenediamine, 1,4,7,10-tetraazacyclododecane-1,4,7,10-tetraacetic acid (DOTA), 1,4,7-triazacyclononanetriacetic acid (NOTA), (1,4,8,11-tetraazacyclotetradecane-1,4,8,11-tetrayl)tetraacetic acid (TETA), 2,2′-(1,4,7,10-tetraazabicyclo[5.5.2]tetradecane-4,10-diyl)diacetic acid (CB-DO2A), 2,2′-(1,4,8,11-tetraazabicyclo[6.6.2]hexadecane-4,11-diyl)diacetic acid (CB-TE2A), diethylene triamine pentaacetic acid (DTPA), 1,4,7,10-tetraazacyclododecane-1,4,7-triacetic acid (DO3A), ethylenediaminetetraacetate (EDTA) or its derivatives, 1,4,7,10-tetraazacyclotridecanetetraacetic acid (TRITA), 1,4,8,11-tetraazacyclotetradecane-1,4,8,11-tetraacetic acid (TETA), 1,4,7,10-tetraazadodecanetetramethylacetate (DOTMA), 1,4,7,10-tetraazadodecane-1,4,7-trimethylacetate (DO3MA), N,N′,N″,N′″-tetraphosphonatomethyl-1,4,7,10-tetraazacyclododecane (DOTP), 1,4,7,10-tetraazacyclododecane-1,4,7,10-tetrakis(methylene methyl-phosphonic acid) (DOTMP), 1,4,7,10-tetraazacyclododecane-1,4,7,10-tetrakis(methylene phenylphosphonic acid) (DOTPP), or N,N′-ethylenedi-L-cysteine, coordinated to a metal selected from Gd⁺³, Eu₊₃, Tm⁺³, Dy⁺³, Yb⁺³, Mn⁺², Fe⁺³, ⁵⁵C, ⁶⁴Cu, ⁶⁷Cu, ⁴⁷Sc, ⁶⁶Ga, ⁶⁸Ga, ⁹⁰Y, ⁹⁷Ru, ^{99m}Tc, ¹¹¹In, ¹⁰⁹Pd, ¹⁵³Sm, ¹⁷⁷Lu, ¹⁸⁶Re, or ¹⁸⁸Re.

In some embodiments, the imaging moiety comprises a derivative of any one of the above listed metal chelators. In some embodiments, the imaging moiety comprises an amide derivative of any one of the above listed metal chelators. In some embodiments, the imaging moiety comprises an ester derivative of any one of the above listed metal chelators.

Structures of metal chelators DOTA, NOTA, DTPA, TETA, CB-TE2A and CB-DO2A:

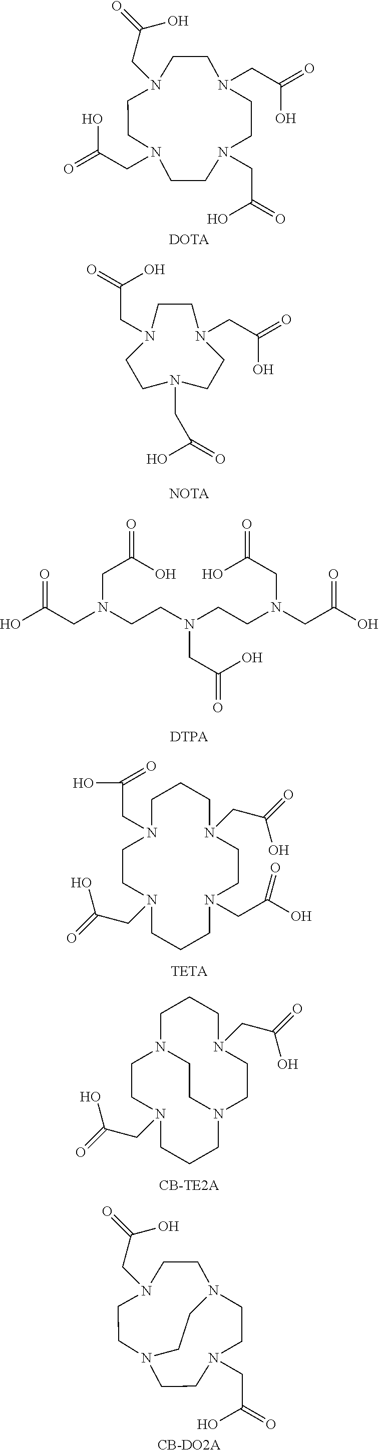

In some embodiments, the imaging agent X comprises Methoxycoumarin, DyLight® 405, HiLyte Fluor™ 405, DyLight® 350, Aminocoumarin, Pacific Blue™, EviTag™ quantum dots-Lake Placid Blue, Cy2®, Chromeo™ 488, DyLight® 488, Alexa Fluor® 488, FAM, Fluorescein Iso-thiocyanate (FITC), EviTag™ quantum dots-Adirondack Green, Chromeo™ 505, HiLyte Fluor™ 488, EviTag™ quantum dots-Catskill Green, Alexa Fluor® 430, Alexa Fluor® 532, HEX, EviTag™ quantum dots-Hops Yellow, Chromeo™ 546, Cy3®, Alexa Fluor® 555, HiLyte Fluor™ 555, 5-TAMRA, Alexa Fluor® 546, DyLight® 549, Phycoerythrin (PE), Tetramethyl Rhodamine Isothiocyanate (TRITC), EviTag™ quantum dots-Birch Yellow, Cy3.5®, Rhodamine Red-X, PE-Dyomics® 590, EviTag™ quantum dots-Fort Orange, ROX, Alexa Fluor® 568, Red 613, Texas Red®, HiLyte Fluor™ 594, PE-Texas Red®, Alexa Fluor® 594, DyLight® 594, EviTag™ quantum dots-Maple-Red Orange, Chromeo™ 494, Alexa Fluor® 633, SureLight® APC, DyLight® 633, Allophycocyanin (APC), Chromeo™ 642, Quantum Red, SureLight® P3, Alexa Fluor® 647, Cy5®, PE-Cy5®, SureLight® P1, PE-Alexa Fluor® 647, PE-Dyomics® 647, DyLight® 649, HiLyte Fluor™ 647, Peridinin Chlorophyll (PerCP), IRDye® 700DX, Alexa Fluor® 660, PE-Cy5.5®, APC-Cy5.5®, Cy5.5®, TruRed, HiLyte Fluor™ 680, Alexa Fluor® 680, DyLight® 680, APC-Cy7®, Cy7®, PE-Dyomics® 747, DyLight® 750, HiLyte Fluor™ 750, PE-Cy7®, IRDye® 800RS, DyLight® 800, IRDye® 800CW.

In some embodiments, the anti-puromycin antibody is conjugated to any of the above imaging agents.

In some embodiments, the imaging moiety Q is Methoxycoumarin, DyLight® 405, HiLyte Fluor™ 405, DyLight® 350, Aminocoumarin, Pacific Blue™, EviTag™ quantum dots-Lake Placid Blue, Cy2®, Chromeo™ 488, DyLight® 488, Alexa Fluor® 488, FAM, Fluorescein Iso-thiocyanate (FITC), EviTag™ quantum dots-Adirondack Green, Chromeo™ 505, HiLyte Fluor™ 488, EviTag™ quantum dots-Catskill Green, Alexa Fluor® 430, Alexa Fluor® 532, HEX, EviTag™ quantum dots-Hops Yellow, Chromeo™ 546, Cy3®, Alexa Fluor® 555, HiLyte Fluor™ 555, 5-TAMRA, Alexa Fluor® 546, DyLight® 549, Phycoerythrin (PE), Tetramethyl Rhodamine Isothiocyanate (TRITC), EviTag™ quantum dots-Birch Yellow, Cy3.5®, Rhodamine Red-X, PE-Dyomics® 590, EviTag™ quantum dots-Fort Orange, ROX, Alexa Fluor® 568, Red 613, Texas Red®, HiLyte Fluor™ 594, PE-Texas Red®, Alexa Fluor® 594, DyLight® 594, EviTag™ quantum dots-Maple-Red Orange, Chromeo™ 494, Alexa Fluor® 633, SureLight® APC, DyLight® 633, Allophycocyanin (APC), Chromeo™ 642, Quantum Red, SureLight® P3, Alexa Fluor® 647, Cy5®, PE-Cy5®, SureLight® P1, PE-Alexa Fluor® 647, PE-Dyomics® 647, DyLight® 649, HiLyte Fluor™ 647, Peridinin Chlorophyll (PerCP), IRDye® 700DX, Alexa Fluor® 660, PE-Cy5.5®, APC-Cy5.5®, Cy5.5®, TruRed, HiLyte Fluor™ 680, Alexa Fluor® 680, DyLight® 680, APC-Cy7®, Cy7®, PE-Dyomics® 747, DyLight® 750, HiLyte Fluor™ 750, PE-Cy7®, IRDye@800RS, DyLight® 800, IRDye® 800CW.

In some embodiments, the anti-puromycin antibody is conjugated to any of the above imaging moieties.

The present invention provides a method of imaging cancer cells in a subject comprising:
1) administering to the subject an effective amount of a compound of the present application, or a pharmaceutically acceptable salt thereof,
   wherein the compound specifically accumulates at cancer cells in the subject and releases puromycin;
2) administering to the subject an amount of an antibody conjugated to a detectable marker, which antibody is capable of specifically binding to the puromycin at the cancer cells in the subject;
3) detecting in the subject the location of the detectable marker; and
4) obtaining an image of the cancer cells in the subject based on the location of the detectable marker in the subject.

The present invention provides a method of detecting cancer cells in a subject comprising:
1) administering to the subject an effective amount of a compound of the present application, or a pharmaceutically acceptable salt thereof,
   wherein the compound specifically accumulates at cancer cells in the subject and releases puromycin;
2) administering to the subject an amount of an antibody conjugated to a detectable marker, which antibody is capable of specifically binding to the puromycin at the cancer cells in the subject;
3) detecting in the subject the location of the detectable marker; and
4) obtaining an image of the cancer cells in the subject based on the location of the detectable marker in the subject, thereby detecting the cancer cells in a subject.

In some embodiments of the above method, the compound specifically accumulates at cancer cells in the subject and releases puromycin into the cancer cells.

In some embodiments of the above method, the compound specifically accumulates at the surface of cancer cells in the subject and releases puromycin onto the surface of the cancer cells.

In some embodiments of the above method, the compound is a puromycin derivative.

In some embodiments of the above method, the compound is a puromycin derivative containing an ε-acetylated lysine moiety.

In some embodiments of the above method, the compound has the following structure:

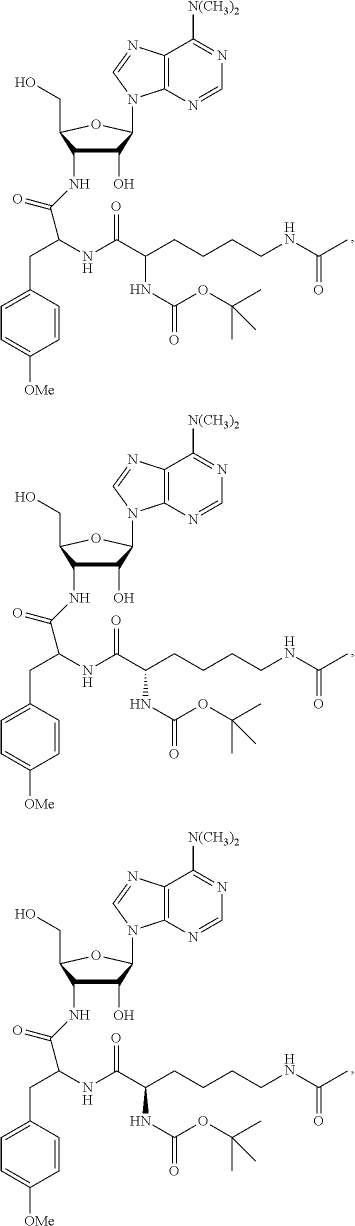

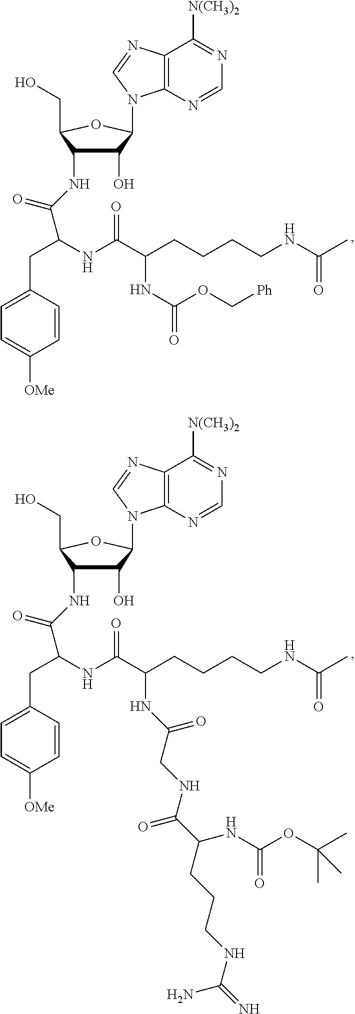

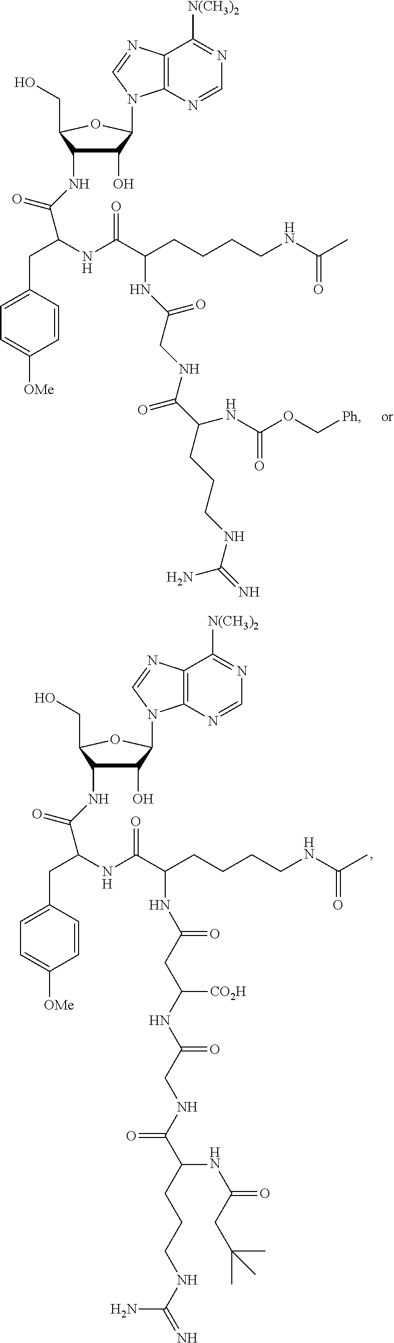

or a pharmaceutically acceptable salt thereof.

In some embodiments of the above method, the compound administered in step 1) specifically accumulates at cancer cells relative to non-cancer cells.

In some embodiments of the above method, detection of of the detectable marker in an organ of the subject is an indication that cancer cells are present in the organ.

In some embodiments of the above method, the cancer cells are lung cancer, breast cancer, prostate cancer, cervical cancer, pancreatic cancer, colon cancer, ovarian cancer, stomach cancer, esophagus cancer, skin cancer, heart cancer, liver cancer, bronchial cancer, testicular cancer, kidney cancer, bladder cancer, spleen cancer, thymus cancer, thyroid cancer, brain cancer, gall bladder cancer, lymphoma or myeloma cells.

In some embodiments of the above method, the cancer cells are cancerous lymphocytes.

In some embodiments of the above method, the cancer cells are cancerous plasma cells.

In some embodiments of the above method, the cancer cells have elevated levels of histone deacetylases or proteases or both.

In some embodiments of the above method, the detectable marker is a fluorescent dye.

In some embodiments of the above method, an in vivo fluorescence imaging device is used to detect the fluorescence emission by the fluorescent dye.

In some embodiments of the above method, an image of the cancer cells is obtained based on the detected fluorescence emission from the fluorescent dye.

In some embodiments of the above method, the detectable marker is an imaging moiety or imaging agent capable of being detected by a molecular imaging device.

In some embodiments of the above method, the detectable marker is the imaging moiety Q as described in any the embodiments contained herein.

In some embodiments of the above method, the detectable marker is the imaging agent X as described in any the embodiments contained herein.

In some embodiments of the above method, the fluorescent dye fluoresces in the visible region, the UV region or near-IR region.

In some embodiments of the above method, the detectable marker is detected using an optical imaging device.

In some embodiments of the above method, the detectable marker is detected using an MRI imaging device or a PET imaging device

In some embodiments of the above method, the optical imaging device is an in vivo fluorescence imaging device.

In some embodiments of the above method, the antibody is an anti-puromycin antibody.

In some embodiments of the above method, the antibody is anti-puromycin antibody, clone 12D10.

In some embodiments of the above method, the detectable marker conjugated to the antibody is Alexa Fluor® 647.

In some embodiments of the above method, the image obtained is a two-dimensional image.

In some embodiments of the above method, the image obtained is a three-dimensional image.

The invention provides a compound having the structure:

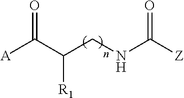

wherein
A is OH, O(C₁-C₆ alkyl) or O(CH₂-aryl);
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

In some embodiments, the compound having the structure:

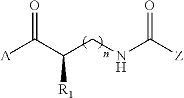

wherein
A is OH, O(C₁-C₆ alkyl) or O(CH₂-aryl);
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

In some embodiments, the compound having the structure:

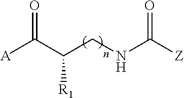

wherein
A is OH, O(C₁-C₆ alkyl) or O(CH₂-aryl);
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
   wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
      wherein an amine of the amino acid or oligopeptide is substituted or unsubstituted; and
n is an integer from 0 to 6;
   or a diastereomer, enantiomer or pharmaceutically acceptable salt of the compound.

As used herein, the term “amino acid” refers to any natural or unnatural amino acid including its salt form, ester derivative, protected amine derivative and/or its isomeric forms. Amino Acids comprise, by way of non-limiting example: Agmatine, Alanine Beta-Alanine, Arginine, Asparagine, Aspartic Acid, Cysteine, Glutamine, Glutamic Acid, Glycine, Histidine, Isoleucine, Leucine, Lysine, Methionine, Phenylalanine, Phenyl Beta-Alanine, Proline, Serine, Threonine, Tryptophan, Tyrosine, and Valine. The amino acids may be L or D amino acids.

As used herein, the term “oligopeptide” refers to a peptide comprising of between 2 and 20 amino acids and includes dipeptides, tripeptides, tetrapeptides, pentapeptides, etc.

An amino acid or oligopeptide may be covalently bonded to an amine of another molecule through an amide linkage, resulting in the loss of an “OH” from the amino acid or oligopeptide.

As used herein, the term “para-aminobenzyl alcohol linker” refers to

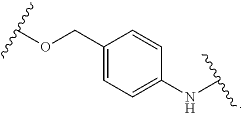 [SMILES: *Nc1ccc(CO*)cc1]

As used herein, the term “therapeutic agent” refers to any agent used to treat a disease or that provides a beneficial therapeutic effect to a subject.

As used herein, the term “chemotherapeutic agent” refers to any agent used to treat cancer or that provides a beneficial therapeutic effect to a subject suffering from cancer.

Certain embodiments of the invention provide compositions or compounds containing therapeutic agents such as a cytotoxin, e.g., a cytostatic or cytocidal agent. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, coichicin, doxorubicin, daunorubicin, dihydroxy anthracin dione, mitoxantrone, mithramycin, actinomycin D, I-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II), (DDP) cisplatin), anthracyclines (e.g., daunorubicin (formerly daunomycin) and doxorubicin), antibiotics (e.g., dactinomycin (fonnerly actinomycin), bleomycin, mithramycin, and anthramycin), and anti-mitotic agents (e.g., vincristine and vinblastine).

Certain embodiments of the invention provide compositions or compounds containing chemotherapeutic agents, which are any agents detrimental to cancer cells. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosinearabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mito-xantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphor-amide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206-1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other chemotherapeutic agents are also within the scope of this invention. Two or more combined compounds may be used together or sequentially.

In some embodiments, the invention provides a method of reducing one or more symptoms of cancer or of imaging cancer cells. Cancers or cells thereof include, but are not limited to, lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. Malignant neoplasms are further exemplified by sarcomas (such as osteosarcoma and Kaposi's sarcoma).

The therapeutic or chemotherapeutic agent is not to be construed as limited to classical chemical therapeutic or chemotherapeutic agents. For example, the agent may be a protein, nucleotide or polypeptide possessing a desired biological activity.

However, the term “therapeutic agent” does not include fluorogenic probes, optical probes, radiolabeled probes, dyes, or other agents that function as imaging or contrast agents.

As used herein, the term “imaging agent” refers to any agent or portion (i.e. imaging moiety) of an agent that is used in medical imaging to visualize or enhance the visualization of the body including, but not limited to, internal organs, cells, cancer cells, cellular processes, tumors, and/or normal tissue. Imaging agents or imaging moieties include, but are not limited to, fluorogenic probes, optical probes, radiolabeled probes, dyes, contrast agents, radioactive contrast agents, MRI contrast agents, PET imaging agents, and SPECT imaging agents. Imaging agents or moieties include, but are not limited to, any compositions useful for imaging cancer cells.

As used herein, the term “histone deacetylase” or “HDAC” refers to any member of the classes of enzymes capable of cleaving an acetyl group (—C(═O)CH₃) from proteins, which include, but are not limited to, histones and microtubules. A histone deacetylase may be zinc-dependent. Examples of HDACs include, but are not limited to, HDAC1, HDAC2, HDAC3, HDAC4, HDAC5, HDAC6, HDAC7, HDAC8, HDAC9, HDAC10, and HDAC11. Additional examples of histone decetylases include, but are not limited to, the Sir2 proteins SIRT1, SIRT2, SIRT3, SIRT4, SIRT5, SIRT6 and SIRT7.

As used herein, “protease” refers to any enzyme that conducts proteolysis.

As used herein, “proteolysis” refers to the hydrolysis or cleavage of a peptide or amide bond.

Antibodies

In some embodiments the anti-puromycin antibody is a monoclonal antibody. Examples include, but are not limited to, Anti-Puromycin Antibody, clone 12D10 (available from Merck Millipore, Billerica, Mass., USA Catalog No. MABE343); Anti-Puromycin, clone 12D10, Alexa Fluor® 488 Conjugate (available from Merck Millipore Catalog No. MABE343-AF488); Anti-Puromycin, clone 12D10, Alexa Fluor® 647 Conjugate (available from Merck Millipore Catalog No. MABE343-AF647); Anti-Puromycin Antibody, clone 4G11 (available from Merck Millipore Catalog No. MABE342); Anti-Puromycin, clone 4G11, Alexa Fluor 488® Conjugate (available from Merck Millipore Catalog No. MABE342-AF488); Anti-Puromycin, clone 4G11, Alexa Fluor 647® Conjugate (available from Merck Millipore Catalog No. MABE342-AF6473); Anti-Puromycin, clone 4G11, Alexa Fluor 647® Conjugate (available from Merck Millipore Catalog No. MABE342-AF647); Anti-Puromycin Antibody, clone 17H1 (available from Merck Millipore Catalog No. MABE341); Anti-Puromycin, clone 17H1, Alexa Fluor® 488 Conjugate (available from Merck Millipore Catalog No. MABE341-AF488); Anti-Puromycin, clone 17H1, Alexa Fluor® 647 Conjugate (available from Merck Millipore Catalog No. MABE341-AF647). Additional commercially available clones may also be used.

The non-conjugated anti-puromycin antibody (including clones 12D10, 4G11 and 17H1) can be conjugated with other fluorescent dyes including Alexa Fluor® 750 using SAIVI™ Rapid Antibody Labeling Kit (Alexa Fluor® 750, available from Life Technologies). These near-infrared dyes can provide much deeper tissue penetration for optical in vivo imaging.

Methods of the present invention relate to the administration of monoclonal antibodies that recognize puromycin incorporated into protein.

An “antibody” as used herein is defined broadly as a protein that characteristically immunoreacts with an epitope (antigenic determinant) of an antigen. As is known in the art, the basic structural unit of an antibody is composed of two identical heavy chains and two identical light chains, in which each heavy and light chain consists of amino terminal variable regions and carboxy terminal constant regions. The antibodies of the present invention include polyclonal antibodies, monoclonal antibodies (mAbs), chimeric antibodies, CDR-grafted antibodies, humanized antibodies, human antibodies, catalytic antibodies, multispecific antibodies, as well as fragments, regions or derivatives thereof provided by known techniques, including, for example, enzymatic cleavage, peptide synthesis or recombinant techniques.

As used herein, “monoclonal antibody” means an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants, each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256:495-97 (1975), or may be made by recombinant DNA methods (see, e.g., U.S. Pat. No. 4,816,567). The monoclonal antibodies may also be isolated from phage display libraries using the techniques described, for example, in Clackson et al., Nature 352:624-28 (1991) and Marks et al., J. Mol. Biol. 222(3):581-97 (1991).

The term “hybridoma” or “hybridoma cell line” refers to a cell line derived by cell fusion, or somatic cell hybridization, between a normal lymphocyte and an immortalized lymphocyte tumor line. In particular, B cell hybridomas are created by fusion of normal B cells of defined antigen specificity with a myeloma cell line, to yield immortal cell lines that produce monoclonal antibodies. In general, techniques for producing human B cell hybridomas, are well known in the art [Kozbor et al., Immunol. Today 4:72 (1983); Cole et al., in Monoclonal Antibodies and Cancer Therapy, Alan R. Liss, Inc. 77-96 (1985)].

The term “epitope” refers to a portion of a molecule (the antigen) that is capable of being bound by a binding agent, e.g., an antibody, at one or more of the binding agent's antigen binding regions. Epitopes usually consist of specific three-dimensional structural characteristics, as well as specific charge characteristics.

Humanized anti-puromycin antibodies are also encompassed by the terms “antibody”. “Humanized antibodies” means antibodies that contain minimal sequence derived from non-human immunoglobulin sequences. For the most part, humanized antibodies are human immunoglobulins (recipient antibody) in which residues from a hyper variable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205, each herein incorporated by reference. In some instances, framework residues of the human immunoglobulin are replaced by corresponding non-human residues (see, for example, U.S. Pat. Nos. 5,585,089; 5,693,761; 5,693,762, each herein incorporated by reference). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331:522-25 (1986); Riechmann et al., Nature 332:323-27 (1988); and Presta, Curro Opin. Struct. Biol. 2:593-96 (1992), each of which is incorporated herein by reference.

Also encompassed by the term “antibody” are xenogeneic or modified anti-puromycin antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598, the entire contents of which are incorporated herein by reference.

Those skilled in the art will be aware of how to produce anti-puromycin antibody molecules of the present invention. For example, polyclonal antisera or monoclonal antibodies can be made using standard methods. To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art. Hybridoma cells can be screened immunochemically for production of antibodies which are specifically reactive with the oligopeptide, and monoclonal antibodies isolated.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

This invention also provides isotopic variants of the compounds disclosed herein, including wherein the isotopic atom is 2H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing 2H or 3H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As sued herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

The term “ester” is intended to a mean an organic compound containing the R—O—CO—R′ group.

The term “amide” is intended to a mean an organic compound containing the R—CO—NH—R′ or R—CO—N—R′R″ group.

The term “phenyl” is intended to mean an aromatic six membered ring containing six carbons and five hydrogens.

The term “benzyl” is intended to mean a —CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

As used herein, “nucleosides” refers to glycosylamines consisting of a nucleobase bound to a ribose or deoxyribose sugar via a beta-glycosidic linkage. Such “nucleosides” may be naturally occurring or synthetic. Examples of such nucleosides are, but not limited to, 5-fluorocytidine and cytarabine.

As used herein, “dexoynucleosides” refers to nucleosides with at least one less oxygen atom. Such “deoxynucleosides” may be naturally occurring or synthetic. Examples of such deoxynucleosides are, but not limited to, 5′-deoxy-5-fluorouridine and 2′-deoxy-5-fluorocytidine.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylaspartamidephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric coated to prevent release of the active ingredient compounds before they reach the small intestine. Materials that may be used as enteric coatings include, but are not limited to, sugars, fatty acids, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

The compounds of the present invention can be synthesized according to general Schemes. Variations on the following general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Materials and Methods

Those having ordinary skill in the art of organic synthesis will appreciate that modifications to general procedures and synthetic routes contained in this application can be used to yield additional derivatives and structurally diverse compounds. Suitable organic transformations are described in in March's Advanced Organic Chemistry: Reactions, Mechanisms, and Structure (Wiley-Interscience; 6^{th} edition, 2007), the content of which is hereby incorporated by reference.

α-Boc-Lys(ε-Ac)—OH and puromycin dihydrochloride were purchased from Bachem and TOKU-E, respectively. 1-ethyl-3-(3-dimethylaminopropyl) carbodiimide hydrochloride (EDC.HCl) and 1-hydroxybenzotriazole (HOBt.H2O) were purchased from Advanced Chem Tech. Dichloromethane (DCM) and dimethylformamide (DMF) were obtained from EMD chemical. DMF was dried and purified by solvent pushstill (SG water USA LLC, Nashua, N.H.). 1H NMR data are reported as chemical shift in ppm (multiplicity, coupling constant in Hz, integration, and tentative assignment. ¹³C NMR data are reported as chemical shift in ppm (tentative assignment). Assignments are based on expected chemical shifts, multiplicities, and coupling constants.

Cell Culture

Colon cancer (Caco-2, COGA-10, HCA-7, HT29, HCT116), normal colon CCD841-CoN, pancreatic cancer (MiaPaca-2, BXPC-3), liver cancer HepG2, and cervical cancer HeLa and normal mouse mammary epithelial Eph4 cell lines were maintained in Iscove's Modified Dulbecco's Medium (IMDM, Invitrogen) supplemented with 10% fetal bovine serum and 100 U/ml penicillin/streptomycin at 37° C. with 5% CO₂ atmosphere. Myeloma cell lines (ARH-77, RPMI8226) were also maintained in Iscove's Modified Dulbecco's Medium (IMDM, Invitrogen) supplemented with 10% fetal bovine serum and 100 U/ml penicillin/streptomycin at 37° C. with 5% CO₂ atmosphere.

Enzymatic Assays

Tissue culture based standard HDAC assays were performed by commercially available fluorimetric HDAC assay kit (SensoLyte, AnaSpec) according to the manufacturer's instruction with modification of using 25 μM Boc-Lys(Ac)-AMC (Bachem) as substrate and measuring parameter (Ex/Em=355 nm/460 nm) for AMC. Basically, HDAC substrate 25 μM Boc-Lys(Ac)-AMC [either with DMSO or 1 μM TSA (Sigma)] was applied to the overnight culture seeded from 6×104 cells in 100 μl medium in 96-well tissue culture plates, followed by 2 to 3 h incubation at 37° C. with 5% CO₂ atmosphere. Then the deacetylation reaction was terminated by addition of HDAC developer solution containing cell lysis buffer and trypsin reagents. After 15 m incubation at room temperature, the fluorescent signal of AMC was measured by SpectraMax M5 microplate reader (Molecular Devices) at Ex/Em=355 nm/460 nm. Live cell lysyl endopeptidase assay was performed by applying the substrate 25 μM Boc-Lys-AMC (Bachem) [either with DMSO or 100 μM Z-FY-CHO (EMD)] to the overnight culture seeded from 6×104 cells in 100 μl medium in 96-well tissue culture plates, followed by 20 h incubation at 37° C. with 5% CO₂ atmosphere, then the fluorescent signal of AMC was measured. Live cell enzymatic assay using Boc-Lys(Ac)-AMC was performed by applying the substrate 25 μM Boc-Lys(Ac)-AMC (either with DMSO, 1 μM TSA or 100 μM Z-FY-CHO) to the overnight culture seeded from 6×104 cells in 100 μl medium in 96-well tissue culture plates, followed by 20 h incubation at 37° C. with 5% CO₂ atmosphere, then the fluorescent signal of AMC was measured. Experiments were repeated at least three times.

Cell Viability Assays

Cell number was determined by cell counting using improved Neubauer hemacytometer. Cell viability was calculated as the number of viable cells divided by the total number of cells using Trypan Blue Stain (Invitrogen) to distinguish non-viable cells. Data were obtained from duplicated samples with quadruplicate measurements. MTS based cell viability assay was performed using CellTiter 96 Aqueous Cell Proliferation Assay (Promega) according to the manufacturer's instruction. Either 1 μl of DMSO alone or variable concentrations of BKAc-Puro or Puro in DMSO were added to the cell lines seeded at 5×104 cells per well in 96-well tissue culture plates in 100 μl of the growth medium, followed by 3-5 d incubation at 37° C. with 5% CO2 atmosphere. Then 20 μl of the MTS reagent was added to each well. After additional 2-3 h incubation, the absorbance of the formazan at 490 nm was measured by the microplate reader. Percent cell viability was expressed relative to the wells containing cells treated with DMSO alone. Data were obtained from triplicate measurements. The IC₅₀ values (the concentration resulting in 50% inhibition) of BKAc-Puro were determined by dose-response curve analysis (GraphPad Prism software). Experiments were repeated at least three times. Determination of non-viable cells using PI staining was performed by adding 1 μg/ml of PI solution (Sigma) to the cell culture, prior to the examination under fluorescence microscopy (Axiovert 3, Carl Zeiss) through a×32 objective equipped with a digital imaging processor (Infinity 3, Lumenera).

In Vivo Assay

To assess the in vivo efficacy of the compounds, a BxPC3 xenograft model is used as an established evaluation method for the anticancer drugs against pancreatic cancer (Kano, M. R. et al. 2007; O'Toole, J. M. et al. 2006). The cells are transfected with expression plasmids for GFP and selected for GFP expression in order to mark the tumor cells. The growth inhibitory effects of the compounds on size-matched BxPC3 xenografts are examined by subcutaneous implantation of BxPC3 cells into nude mice. BxPC3 cells are injected subcutaneously and allowed to grow for 2-3 weeks to reach proliferative phase before initiation of drug administration. Mice (5 animals per group) are treated with the vehicle (PBS), the various prodrugs, or 5-FU by intraperitoneal administration every 3 days for a total of 4 doses. The prodrugs are expected to have lower toxicity than the parental 5-FU, thus dose ranges that have been established by others for 5-FU are used (Overholser, J. P. et al. 2000). Then, the mice are imaged over a 3 week period using the Maestro small animal imaging system. This scanner captures tumor growth in the mice by detection of increasing area and intensity of green fluorescence emitted from the GFP. Xenograft tumors are measured externally every second day until day 16-21, and tumor volumes are approximated by using the equation vol=(a×b²)/2, where vol, a, and b represent volume, the length of the major axis, and the length of the minor axis, respectively. Relative tumor volume is calculated by dividing tumor volume by that on day 0 (the day of treatment initiation). The weight of the mice is checked to monitor unfavorable effects by the compounds being tested and upon any indication of distress the mice is humanely euthanized. Statistical significance of the data is evaluated by performing one-way ANOVA with post hoc Turkey's test to compare means (GraphPad Prism software). The efficacy of the compounds is further evaluated in a similar manner by using other pancreatic adenocarcinoma cell lines CFPac-1 and MiaPaCa-2 and the PDA lines in which HDAC3 levels have been reduced.

To assess the in vivo efficacy of the compounds, mouse xenograft model using HCT116 colon cancer cells is used as an established evaluation method for the anticancer drugs against cancer (Cao, Z. A. et al. 2006). The growth inhibitory effects of the compounds on size-matched xenografts are examined by subcutaneous implantation of HCT116 cells into nude mice. HCT116 cells are injected subcutaneously and allowed to develop palpable tumors (50-150 mm³) before initiation of drug administration. Mice (5-10 animals per group) are treated with the vehicle (DMSO or PBS) or the prodrugs by intraperitoneal administration. Dose ranges are determined by the dose escalation studies of the prodrugs. Tumor volumes are approximated by using the equation vol=(a×b²)/2, where vol, a, and b represent volume, the length of the major axis, and the length of the minor axis, respectively. Relative tumor volume is calculated by dividing tumor volume by that on day 0 (the day of treatment initiation). The weight of the mice is checked to monitor unfavorable effects by the compounds being tested and upon any indication of distress the mice is humanely euthanized. Statistical significance of the data is evaluated by performing one-way ANOVA with post hoc Turkey's test to compare means (GraphPad Prism software). The efficacy of the compounds is further evaluated in a similar manner by using other cancer cell lines.

Athymic mice (NCr, female, age 6 weeks, Taconic) were subcutaneously injected with 5×10⁵ cells (HCT116 or HT29) into the lower flank, then treatment was initiated when small palpable tumors had developed (>3 mm in diameter). Either Boc-KAc-Puro in 150 mM HCl (pH 2 adjusted by NaOH) or acidified saline (pH 2) as control was daily administered intraperitoneally for 10 d. Tumor volumes and body weight were monitored daily. Tumor volume was estimated by the equation vol=(a×b²)/2, where vol, a, and b represent volume (mm3), the length of the major axis (mm), and the length of the minor axis (mm), respectively. Data were expressed as mean±s.d.

Statistical Analyses

For the in vivo experiments, a two-tailed student's t test was used to calculate statistical significance of the observed differences. P<0.05 was considered statistically significant.

Immunological Detection of Puro-Labeled Proteins

Cultured cells or tissue samples were sonicated in RIPA lysis buffer (1% NP-40, 0.1% SDS, 50 mM Tris-HCl pH 7.4, 150 mM NaCl, 0.5% sodium deoxycholate, 1 mM EDTA) supplemented with PMSF and protease inhibitor cocktail (Roche) on ice followed by clarification. Protein lysates were separated on sodium dodecyl sulfate-polyacrylamide gels and transferred onto nitrocellulose membranes (Whatman). The primary antibodies used were anti-puromycin (3RH11, KeraFAST) and anti-α-tubulin (Sigma-Aldrich). Proteins of interest were detected with anti-mouse secondary antibody by chemiluminescence (ECL Kit, GE Healthcare Life Sciences).

Live Single Cell Imaging of Cancer by Fluorescence Microscopy

Cell surface detection of Puro by anti-puro antibody (12D10) conjugated to Alexa 647 (EMD Millipore). ARH-77 cells were treated with Puro (0.25 μg/ml) or Boc-KAc-Puro (20 μg/ml) for 16 hr then harvested by centrifugation followed by two times wash using cold washing solution (0.1% BSA in PBS). Cells were resuspended in the same solution and incubated 30 min on ice with Alexa 647 conjugated mouse IgG2a anti-puromycin antibody (12D10). Cells were washed twice with the solution followed by fluorescence microscope analysis. The specificity of the antibody was confirmed by using isotype control antibody (mouse IgG2a conjugated with Alexa 647).

Live Single Cell Detection of Cancer by Fluorescence Activated Cell Sorting (FACS)

Cell surface detection of Puro by anti-puro antibody (12D10) conjugated to Alexa 647 (EMD Millipore). RPMI8226 cells were treated with Puro (0.25 μg/ml) or Boc-KAc-Puro (20 μg/ml) for 16 hr then harvested by centrifugation followed by two times wash using cold washing solution (0.1% BSA in PBS). Cells were resuspended in the same solution and incubated 30 min on ice with Alexa 647 conjugated mouse IgG2a anti-puromycin antibody (12D10). Cells were washed twice with the solution followed by FACS analysis (BD FACSCalibur). The specificity of the antibody was confirmed by using isotype control antibody (mouse IgG2a conjugated with Alexa 647).

### Example 1

### Levels of HDAC in Cancer Cells

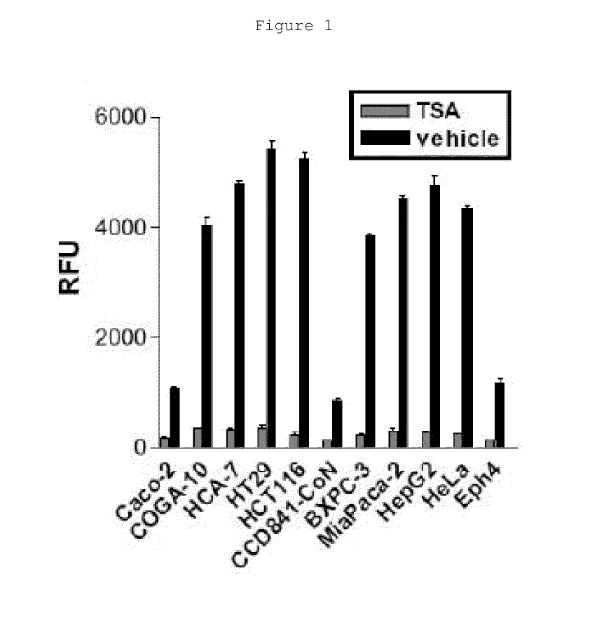

To test the hypothesis that cancer cells have elevated levels of HDAC activity, HDAC activity was measured in a panel of human cancer cell lines including colon cancer (Caco-2, COGA-10, HCA-7, HT29, HCT116), pancreatic cancer (BXPC-3, MiaPaca-2), liver cancer (HepG2), and cervical cancer (HeLa), as well as non-tumorigenic human colon epithelial cells (CCDB41-CoN) and normal mouse mammary epithelial cells (Eph4) (Mariadason, J. M. et al. 2001; Wegener, D. et al. 2003) (FIG. 1).

Using a standard fluorometric assay, marked high levels of HDAC activity was observed in all malignant cell lines tested but, in clear contrast, low levels in less-tumorigenic and normal cell lines (Caco-2, CCD841-CoN, Ehp4). The specificity of the HDAC activity was confirmed by a HDAC inhibitor Trichostatin A (TSA) (Yoshida, M. et al. 1990). Although Caco-2 cells originated from human colon adenocarcinoma, these cells are known to be less-tumorigenic and behave like normal, differentiated enterocytes in vivo when cultured as confluent cells (Mariadason, J. M. et al. 2001). These results supported our approach to target HDAC activity for cancer therapy and lead us to explore the possibility of designing new anticancer agents by incorporating chemical HDAC substrates into cytotoxic drugs so that their cytotoxicity can be selectively activated in cancer cells in a HDAC dependent manner.

### Example 2

### Boc-Lys(Ac)-AMC

Typical fluorogenic substrates for class I HDACs are comprised of an ε-acetylated lysine residue (alone or in short peptides) coupled to a fluorophor moiety AMC (7-amino-4-methylcoumarin) such as cell-permeable Boc-Lys(Ac)-AMC (Wegener, D. et al. 2003; Bonfils, C. et al. 2008). The standard HDAC assay is based on the two-step conversion of the substrate: 1) HDAC-dependent deacetylation of ε-acetylated lysine, 2) protease-dependent cleavage of Lys-AMC amide bond and subsequent release of free fluorophor AMC, which fluoresces (Wegener, D. et al. 2003). In the tissue culture based assays, the second step is normally performed following cell lysis by addition of excessive amounts of trypsin, which only recognizes and processes deacetylated form of lysine. Given that the Lys-AMC amide bond can be cleaved by other endogenous proteases commonly associated with cancer cells such as lysosomal proteases (Weissleder, R. et al. 1999), it was hypothesized that the second step can occur in live cells without cell lysis and trypsin treatment.

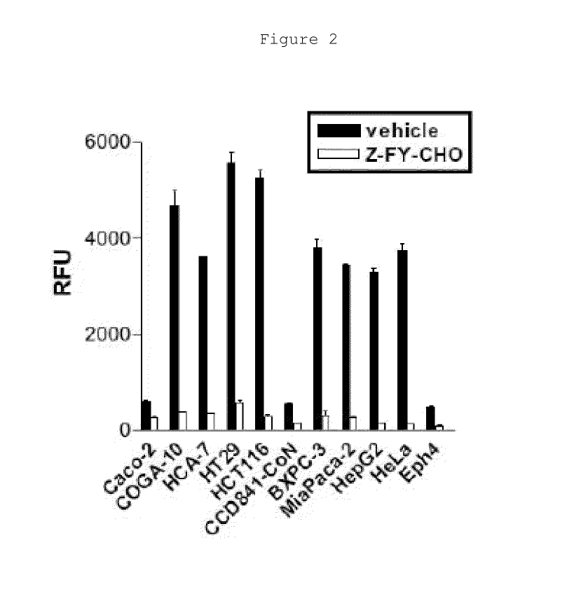

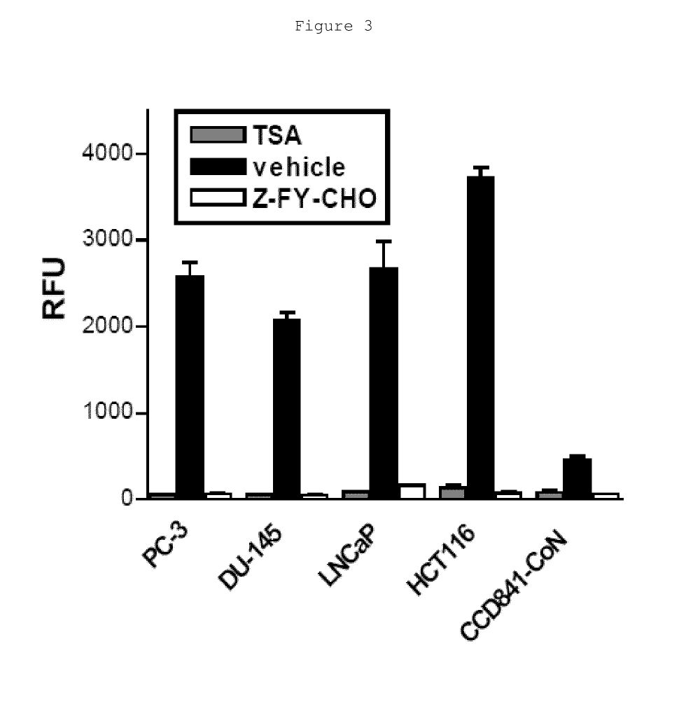

To test this possibility, levels of lysyl endopeptidase activity in live cells using Boc-Lys-AMC as a substrate was assessed. As shown in FIG. 2, the levels of protease activity were much greater in the malignant cells, whereas those in the less-tumorigenic Caco-2, CCD841-CoN and Eph4 cells were only basal, showing striking correlation between elevated HDAC and lysyl endopeptidase activities in the malignant cells. These results prompted identification of the endogenous protease releasing AMC in live cells. Considering the mode of substrate recognition and elevated activity in malignant cells, Z-FY-CHO, a specific inhibitor of lysosomal cysteine protease catepsin L (CTSL), was tested (Gonzalez-Suarez, I. et al. 2011). CTSL is known to play crucial roles at multiple stages of tumor progression and metastasis (Lankelma, J. M. et al 2010; Jedeszko, C. & Sloane, B. F. 2004). Furthermore, there is growing evidence that upregulation of CTSL is a hallmark of metastatic cancers and could be utilized as a prognostic marker (Gonzalez-Suarez, I. et al. 2011; Joyce, J. A. et al. 2004). In all cases tested, the release of AMC was significantly blocked by Z-FY-CHO (FIGS. 2 & 3), indicating that CTSL is responsible for the second step reaction in the live cell assay.

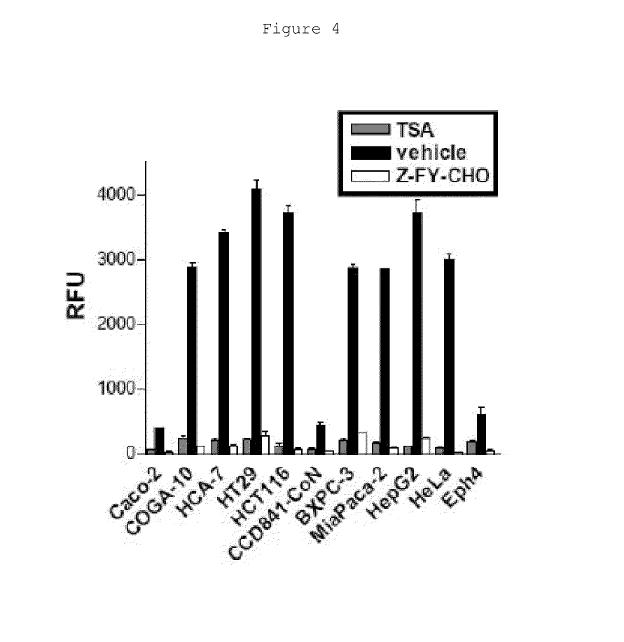

To confirm if the two-step conversion of the HDAC substrate seamlessly proceeded in live cells, the levels of released AMC using Boc-Lys(Ac)-AMC was monitored (FIG. 4). Consistent with the levels of HDAC and CTSL activities, selectively higher levels of AMC were detected in all malignant cell lines tested. The observed effects shown in the presence of TSA or Z-FY-CHO confirmed that both HDAC and CTSL activities were required for the release of AMC in live cells (FIG. 4). The data clearly indicated that the HDAC substrate can be selectively processed in live cells, further supporting the approach to create HDAC activity based anticancer agents.

### Example 3

### Boc-Lys(Ac)-AMC vs. Ac-Arg-Gly-Lys(Ac)-AMC

Commercially available fluorescent compounds were used to measure HDAC activity in cells to obtain important preliminary data. Established substrates for the sensitive fluorogenic assay of class I HDACs are comprised of a short peptide sequences coupled with an ε-acetylated lysine residue followed by a fluorophor moiety (Wegener, D. et al 2003). Such substrates include Boc-Lys(Ac)-X and Ac-Arg-Gly-Lys(Ac)-X, where X represents fluorophor moiety such as AMC. The assay was also based on the two-step conversion of the fluorogenic peptide substrate: 1) Intracellular HDAC-dependent deacetylation of ε-acetylated lysil moiety, 2) protease-dependent cleavage of Lys-X amide bond and subsequent release of free fluorophor. The second step required cell lysis followed by addition of excessive amounts of exogenous proteases (commonly trypsin) to the reaction.

Fluorescence measurement was done at λₑₓ=390 nm and λₑₘ=460 nm for AMC. FIG. 4A shows the standard assay using these compounds, which were incubated with cells for various times, in this case 2.5 hr, during which time they were readily taken up by the cells and the endogenous HDAC enzymes deacetylate the lysine residues. Cells were then lysed and the lysates incubated with exogenously added excess trypsin to cleave only those lysine residues that are de-acetylated and activate fluorescence. As tumor cells are known to frequently exhibit increased levels of intracellular protease activity, it was postulated that intracellular tumor-associated protease activity would be able to cleave the exposed lysine residue in live cells and release the fluorescent compounds. This live cell reaction would clearly not be indicative of total cellular HDAC activity as it would now also depend on access of the compounds to cellular proteases and also require the proteases to be able to cleave the lysine in its specific context.

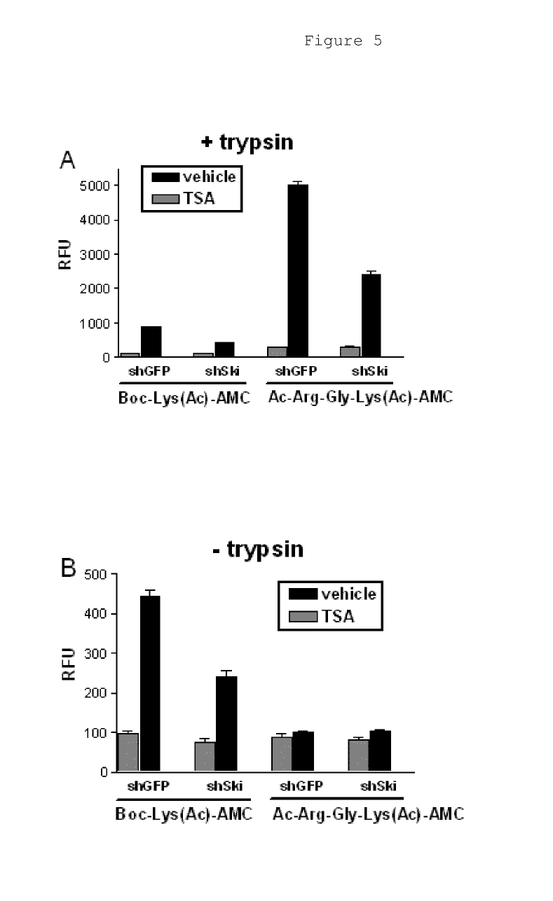

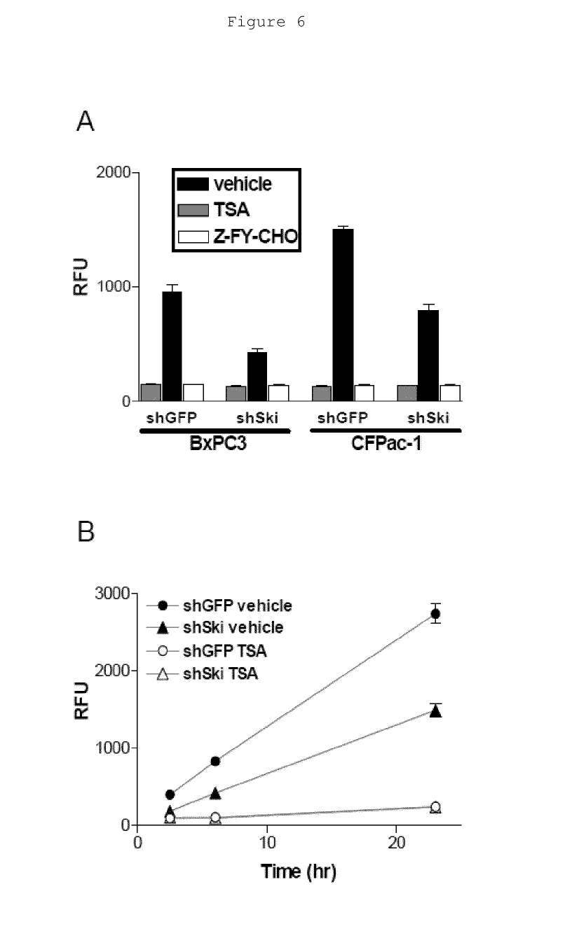

Nevertheless if fluorescence was activated it would indicate that the live PDA (Pancreatic ductal adenocarcinoma) cells had the necessary enzymatic activities to activate these compounds and provide key evidence that they should also be able to activate the pro-drugs described above. FIG. 5 shows a comparison of the normal assay (A) in which cells are lysed and then incubated with trypsin with a similar experiment (B) in which live cells were incubated with the same fluorescent compounds. Two substrates were compared, one substrate that just had a single acetylated lysine residue, Boc-Lys(Ac)-AMC, with one that has a three amino acid peptide, Ac-Arg-Gly-Lys(Ac)-AMC. Note the scales on the two graphs differ by a factor of ten. As can be seen in FIG. 5A, both compounds measured total HDAC activity, albeit with differing sensitivities, and again showed that Ski knockdown by shRNA against Ski lowers the HDAC activity. More importantly as shown in FIG. 4B, in which live cells were incubated with the two substrates, the Boc-Lys(Ac)-AMC was activated in a HDAC and Ski-dependent manner to approximately 50% of total HDAC activity, whereas the other substrate was not activated at all. This experiment demonstrated that live PDA cells have the necessary enzymatic activities (HDAC plus proteolytic) to activate acetylated-lysine substrates and that the context of the acetylated-lysine was important for the proteolytic cleavage activation step. This data also indicated that it should be possible to synthesize prodrugs of similar structures that can deliver cytotoxic compounds inside cells whose activation is dependent on intracellular HDAC and protease activities and the drugs can be varied in both payload and peptide side chains to generate maximal tumor specific activity.

To address possible intracellular proteases that may be responsible for the activation of the substrate it was noted that a lysosomal cystein protease cathepsin L has been studied as a tumor associated protease, and it's utility as a prognostic marker has been evaluated for numerous different tumor types including pancreatic cancers (Joyce, J. A. et al. 2004; Von Burstin, J. et al. 2008; Niedergethmann, M. et al. 2004). Thus, a highly specific inhibitor of cathepsin L, Z-FY-CHO was tested in the live cell assay (Pacheco, F. J. et al. 2005). As shown in FIG. 6A, incubation of the cells with the cathepsin L inhibitor completely abrogated the activation of the substrate. Thus, cathepsin L was targeted as a candidate enzyme for the second necessary step in the prodrug activation reaction. This experiment also confirmed that both HDAC and protease activities are required for activation as HDAC inhibition (TSA) and protease inhibition (Z-FY-CHO) completely inhibited activation. In addition, a time course experiment showed that AMC was released linearly over the time (20 hr), while in the presence of TSA, AMC release was inhibited (FIG. 6B), demonstrating that cathepsin L hardly cleaves Boc-Lys(Ac)-AMC even after longer incubation periods.

### Example 4

### Boc-Lys(Ac)-Puromycin

Synthesis Boc-Lys(Ac)-Puromycin

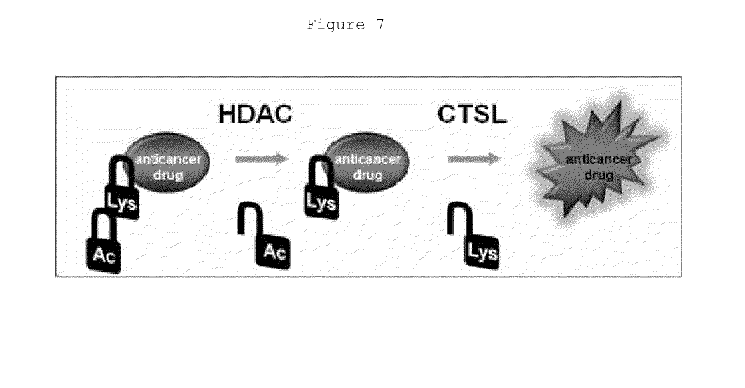

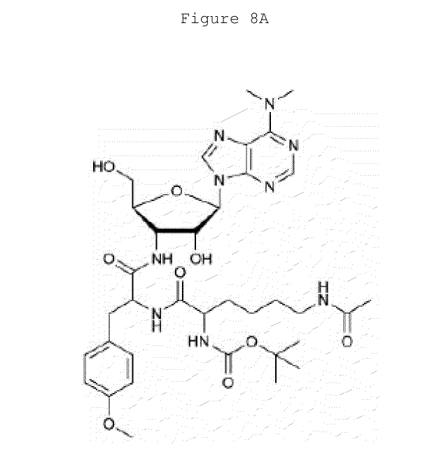

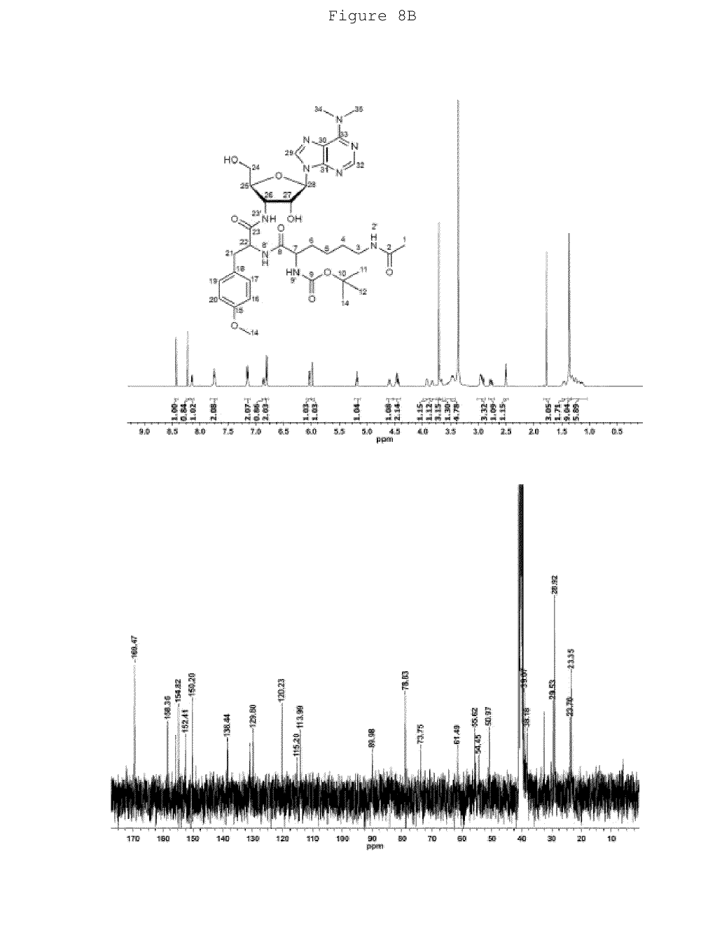

Having established the utility of the HDAC substrate Boc-Lys(Ac)-AMC in live cells, a compound was synthesized by applying the HDAC substrate into cytotoxic drugs (FIG. 7). Among many cytotoxic drugs, puromycin (Puro), an aminonucleoside antibiotic produced by the bacterium Streptomyces alboniger and a potent protein synthesis inhibitor that causes premature termination of growing polypeptide chains on ribosome was selected (Vara, J. A. et al. 1986). Puro is a potent translational inhibitor in both prokaryotic and eukaryotic cells. Resistance to Puro is conferred by the puromycin N-acetyltransferase gene, pac, which is widely used as a selection marker for gene transfer (Vara, J. A. et al. 1986). Given that the cytotoxicity of Puro is completely inactivated by the acetylation of its amino group by the pac gene product, it was reasoned that this antibiotic would be an ideal candidate for the evaluation of the HDAC dependent release of cytotoxicity. Thus, Boc-Lys(Ac)-puromycin (BKAc-Puro) was synthesized by conjugating Boc-Lys(Ac)—OH with the amino group of Puro (FIG. 8).

N-[3′-(α-Amino-p-methoxyhydrocinnamamido)-3′-deoxy-N,N-dimethyldenosinyl)-N-α-(t-butoxycarbonyl]-N-ε-acetyl-L-lysineamide (Boc-Lys(Ac)-Puromycin): A mixture of puromycin dihydrochloride (50 mg, 0.092 mmol), α-Boc-Lys(ε-Ac)—OH (31 mg, 0.11 mmol), EDC.HCl (21 mg, 0.11 mmol), HOBt.H₂O (17 mg, 0.11 mmol), and DIEA (N,N-diisopropylethylamine) (18 μL, 0.11 mmol) was stirred in DMF (10 ml) at rt for 18 h. After concentrating the solution under reduced pressure, the residue was dissolved in DCM. The DCM solution was washed with H₂O three times, dried over Na₂SO₄, and concentrated. The crude oil was purified by silica gel column chromatography using a linear gradient from 5 to 10% MeOH in DCM and dried to yield α-Boc-Lys(ε-Ac)-Puromycin as a white solid (55 mg, 75% yield): mp: 182-183° C.; ¹H NMR (500 MHz, DMSO) δ 8.43 (s, 1H, H-29), 8.22 (s, 1H, H-32), 8.15 (d, J=7.7 Hz, 1H, H-8′), 7.74 (d, J=7.1 Hz, 2H, H-2′, H-23′), 7.15 (d, J=8.4 Hz, 2H, H-17), 6.86 (d, J=8.3 Hz, 1H, H-9′), 6.80 (d, J=8.6 Hz, 2H, H-16, H-20), 6.03 (d, J=4.6 Hz, 1H, H-28), 5.98 (d, J=2.8 Hz, 1H, H-27), 5.18 (t, J=5.4 Hz, 1H, H-22), 4.60 (dd, J=13.6, 8.1 Hz, 1H, H-7), 4.52-4.39 (m, 2H, H-25, H-26), 3.98-3.76 (m, 2H, H-24), 3.70 (s, 3H, H-14), 3.69-3.40 (m, 6H, H-34, H-35), 3.02-2.86 (m, 3H, H-3, H-21), 2.76 (dd, J=13.7, 8.7 Hz, 1H, H-21), 1.77 (s, 3H, H-1), 1.52-1.38 (m, 2H, H-6), 1.38-1.32 (s, 9H, H-11, H-12, H-13), 1.33-1.05 (m, 6H, H-4, H-5) ppm. ¹³C NMR (101 MHz, DMSO) δ 169.5 (C-8, C-23), 158.4 (C-9), 155.8 (C-2), 154.8 (C-33), 152.4 (C-32), 150.2 (C-31), 138.4 (C-29), 138.3 (C-15), 131.0 (C-17, C-19), 129.8 (C-18), 120.2 (C-30), 114.0 (C-16, C-20), 90.0 (C-28), 78.8 (C-10, C-25), 73.8 (C-27), 61.5 (C-24), 55.6 (C-26), 55.3 (C-7), 54.5 (C-22), 51.0 (C-14), 40 ((C-34, C-35, underneath DMSO peak), 39.1 (C-3), 38.2 (C-21), 32.5 (C-6), 29.5 (C-4), 28.9 (C-11, C-12, C-13), 23.7 (C-5), 23.4 (C-1); MS (m/z): [M]⁺ calcd. for C₃₅H₅₁N₉O₉, 742.38. found, 742.47.

Biological Data

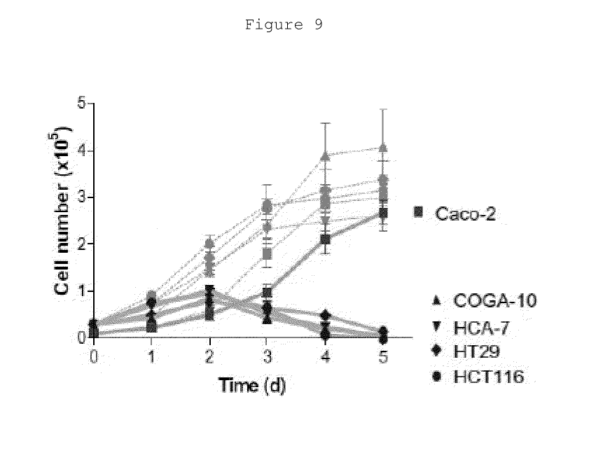

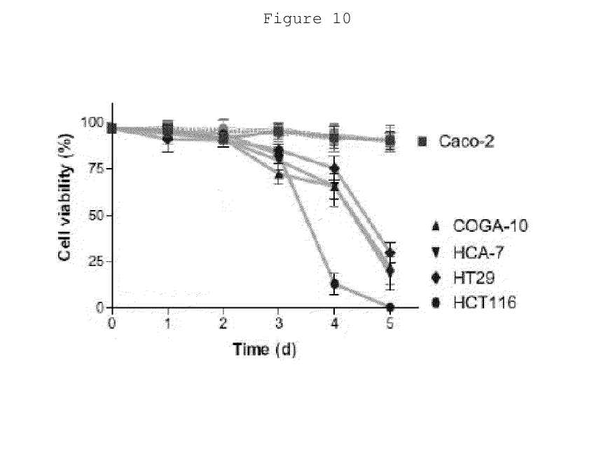

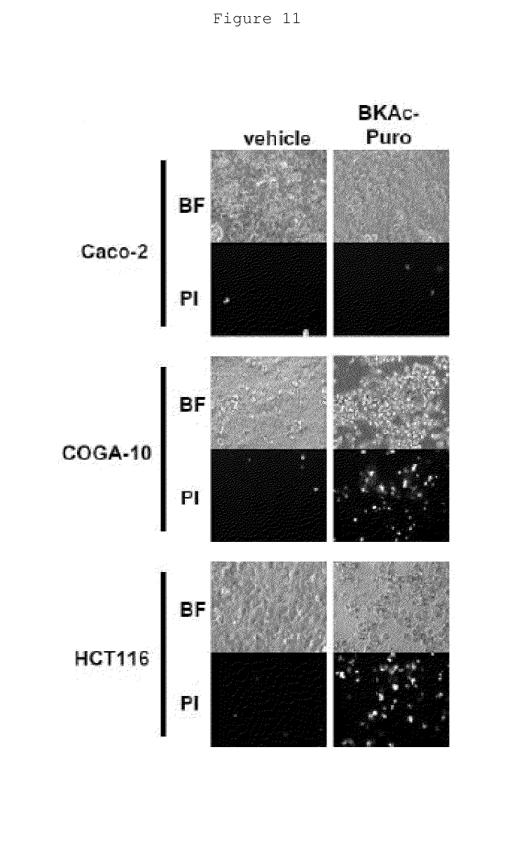

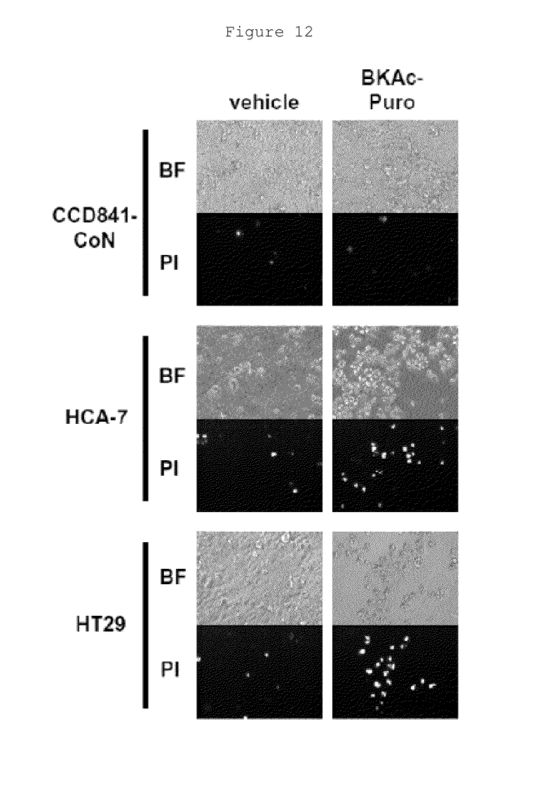

Anticancer properties of BKAc-Puro in a panel of colon cell lines were assessed. The agent impaired the proliferation of all four malignant cells with high HDAC and CTSL activity (FIG. 9), while in non-malignant Caco-2 cells, it allowed them to proliferate to confluency, showing a noteworthy feature distinguished from the general cytostatic effect on normal cells by most widely used antimetabolite cancer drugs and HDACi (Mark, P. A. & Xu, W. S. 2009). Consistent with the effect on cell proliferation, massive cell death was detected only in the malignant cells as judged by trypan blue exclusion (FIG. 10), morphological changes under phase contrast and propidium iodide (PI) staining (FIGS. 11-12). The results indicated that the antiproliferative and cytotoxic effects of BKAc-Puro was selective to malignant cells and exclusively associated with HDAC activity.

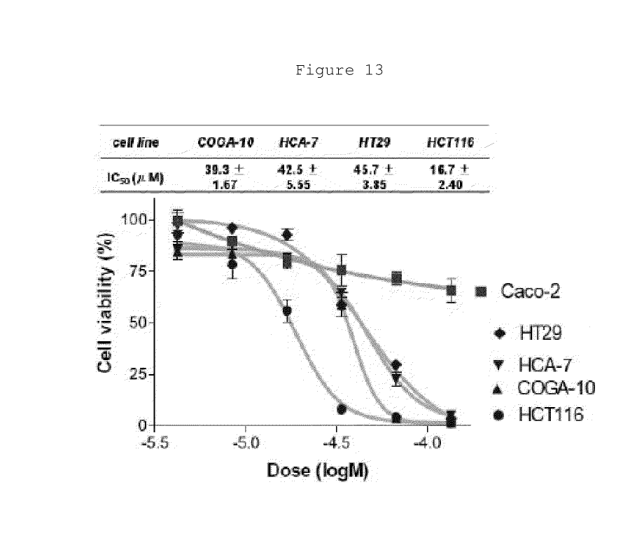

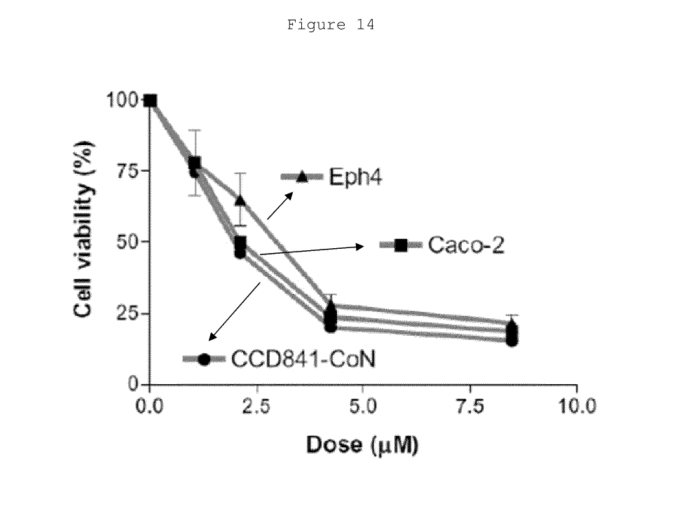

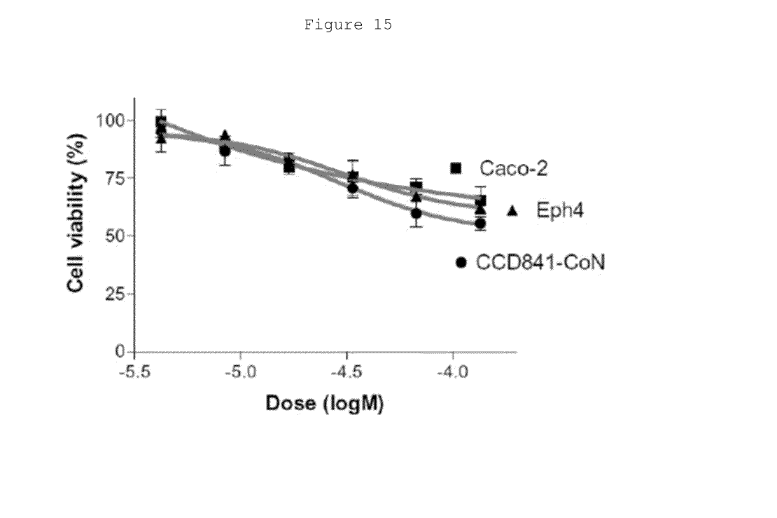

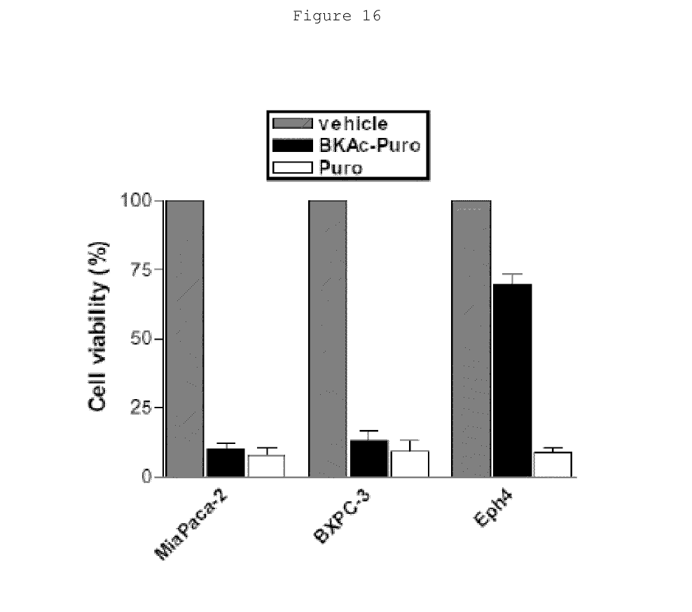

To further evaluate the anticancer efficacy of BKAc-Puro, cell viability assays using MTS, 3-(4,5-dimethylthiazol-2-yl)-5-(3-carboxymethoxyphenyl)-2-(4-sulfophenyl)-2H-tetrazolium were performed (FIG. 13). The dose-response curves obtained from the panel of colon cell lines indicate comparable values of IC₅₀ (10¹ μM range) for the malignant cell lines with high HDAC and CTSL activity. In striking contrast, the IC₅₀ values for non-malignant Caco-2 cells were not available, as sufficient inhibition of cell viability was not achieved with doses up to 135 μM, more than 30 times higher dose of parental Puro effective to these cells (FIG. 14). Normal cell lines with low HDAC activity (CCD841-CoN and Eph4) showed similar pattern of dose-response to Caco-2 (FIG. 15), proving that the observed inactivity of the agent is not limited to Caco-2. Furthermore, the BKAc-Puro was also effective to other malignant cell lines including pancreatic cancer (FIG. 16), consistent with their increased HDAC and CTSL activities (FIGS. 1-3), suggesting that the agent could be effective to a broad spectrum of cancers. Taken together, these results demonstrated that the BKAc-Puro exhibits high grade of selectivity toward cells with high HDAC and CTSL activity, which is the characteristic of malignant cancer, while securing tight protection to non-malignant and normal cells with low (basal) HDAC and CTSL activity.

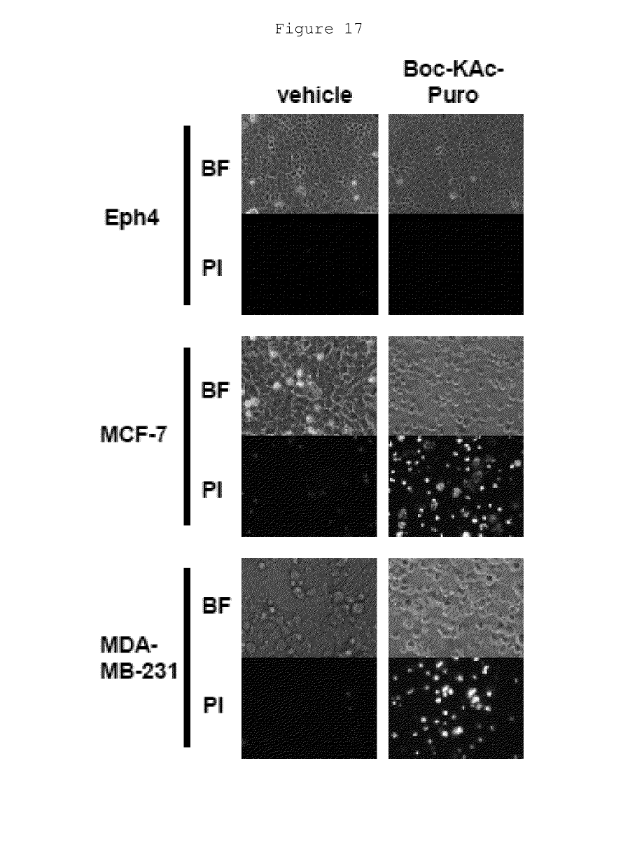

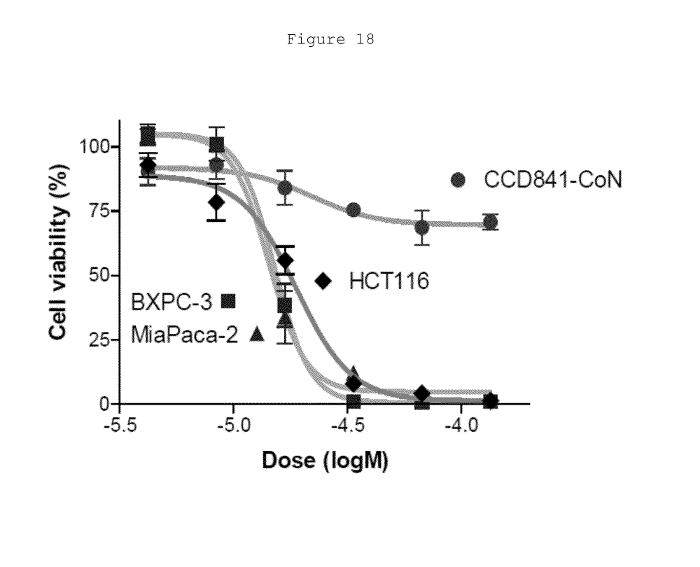

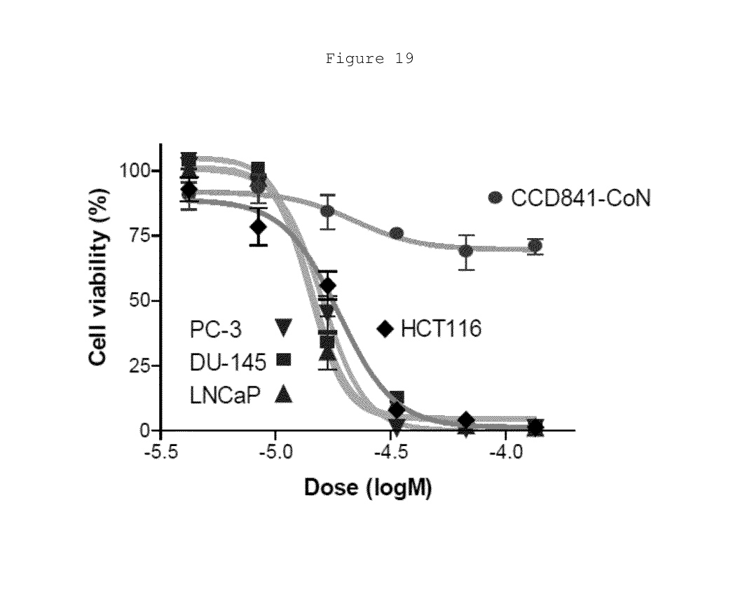

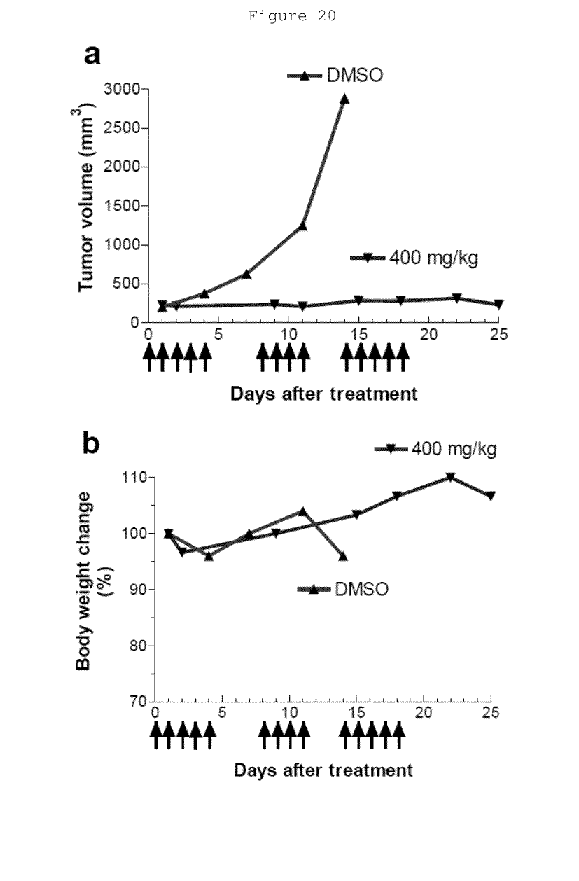

BKAc-Puro selectively caused cell death in breast cancer cells (MCF-7 and MDA-MB-231) while leaving normal mammary gland epithelial cells (Eph4) unharmed (FIG. 17). BKAc-Puro effectively caused cell death in pancreatic cancer cells (BXPC-3 and MiaPaca-2) that are known to resistant to conventional chemotherapeutic drugs including 5-FU and Gemicitabine while having relatively little effect on normal colon cells unharmed (FIG. 18). BKAc-Puro effectively caused cell death in prostate cancer cells (PC-3, DU-145 and LNCaP) while having relatively little effect on normal colon cells unharmed (FIG. 19).

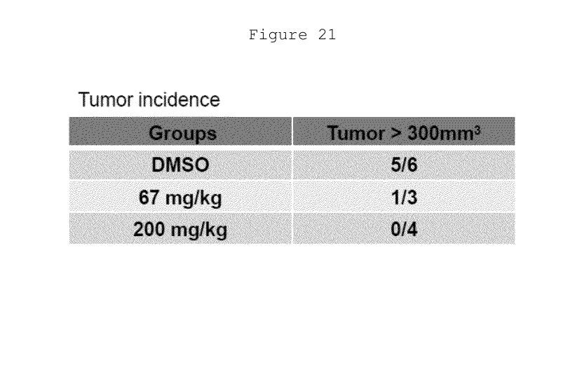

In vivo anticancer efficacy of BKAc-Puro was demonstrated by the inhibition of tumor growth in a mouse xenograft model. No toxicity was observed in the group treated with 400 mg/kg of the drug during and after 14 injections (FIG. 20A-B). Further, in vivo anticancer efficacy of BKAc-Puro was demonstrated by the inhibition of tumor incidence in mouse xenograft model (FIG. 21). When treated with 200 mg/kg, no tumors greater than 300 mm² were observed. Consistent with the data presented above, no toxicity was observed in the groups treated with the drug.

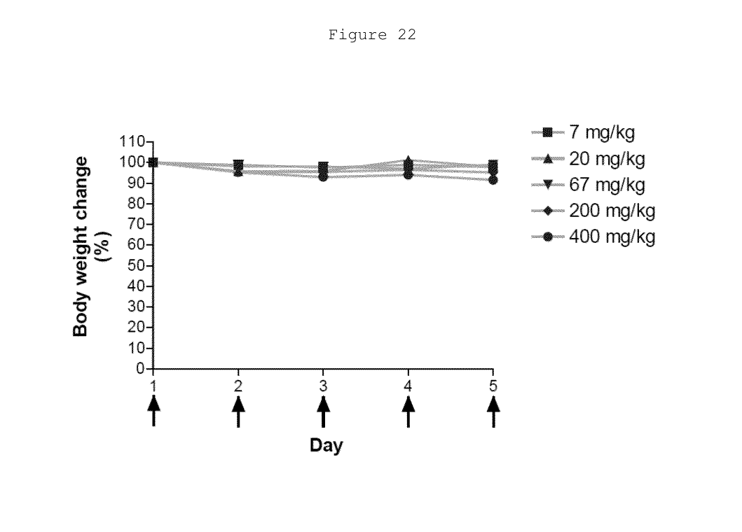

No severe systemic toxicity was observed up to 400 mg/kg from 5 consecutive daily injections (FIG. 22). Considering that the LD50 values for single administration of unmasked puromycin are 335 mg/kg (intravenously), 580 mg/kg (intraperitoneally), and 720 mg/kg (orally) [ABANAE Antibiotics Annual, 1954/1955], BKAc-Puro appears to be well-protected and well-tolerated in animals.

### Example 5

### Boc-Lys(Ac)-5-Fluorocytidine

Synthesis of Boc-Lys(Ac)-5-fluorocytidine

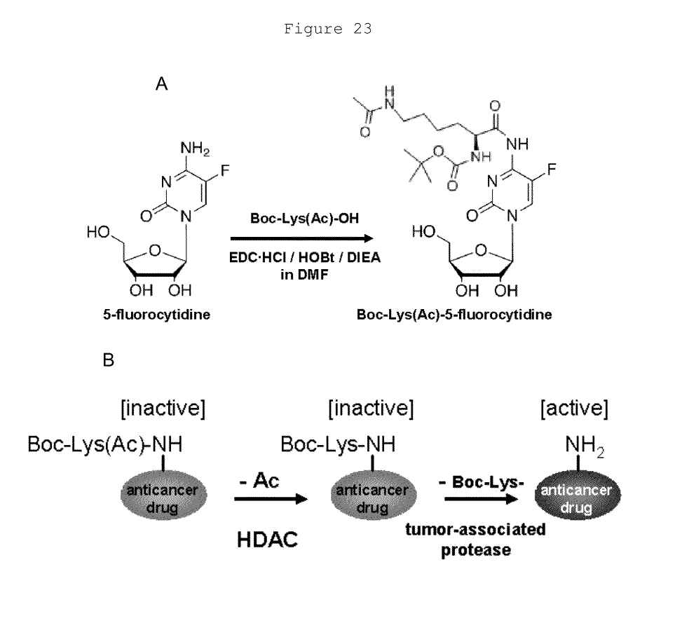

Boc-Lys(Ac)-5-fluorocytidine (BKAc-5FCR) was synthesized by conjugating Boc-Lys(Ac)—OH with the amino group of 5-fluorocytidine (5FCR) (FIG. 23A).

t-butyl (6-acetamido-1-((1-((2R,3R,4S,5R)-3,4-dihydroxy-5-(hydroxymethyl)tetrahydrofuran-2-yl)-5-fluoro-2-oxo-1,2-dihydropyrimidin-4-yl)amino)-1-oxohexan-2-yl) carbamate (Boc-Lys(Ac)-5-fluorocytidine): A mixture of 5-fluorocytidine, N-α-(t-butoxycarbonyl)-N-ε-acetyl-L-lysine (Boc-Lys(Ac)—OH), 1-ethyl-3-(3-dimethylaminoproryl) carbodiimide hydrochloride (EDC.HCl), 1-hydroxybenzotriazole (HOBt), and N,N-diisopropylethylamine were stirred in DMF (dimethylformamide) for 18 h. Water was then added and the organic soluble part was extracted with methylene chloride. Then the compound was purified by silica gel column chromatography using 5-10% MeOH in methylene chloride and dried to give Boc-Lys(Ac)-5-fluorocytidine.

Biological Data

The in vitro efficacy of the compounds in a series of BxPC3 and CFPac-1 derivatives with reduced Class I HDAC activity following Ski knockdown (shSki), HDAC3 knockdown and control (shGFP) was assessed. The growth inhibitory/viability effects of the compounds (IC₅₀ value, the concentration resulting in 50% growth inhibition) on the various cell lines were determined by dose response curve analysis (GraphPad Prism software) of MTT [3-(4,5dimethylthiazol-2-yl)-2,5-diphenyl-tetrazolium bromide] cell viability assay. If the prodrugs are activated in an HDAC-dependent manner, the cells with reduced HDAC activity would be predicted to be more resistant to the prodrug treatment than control cells, while the effect of free parental drugs on both cells would be expected to be similar.

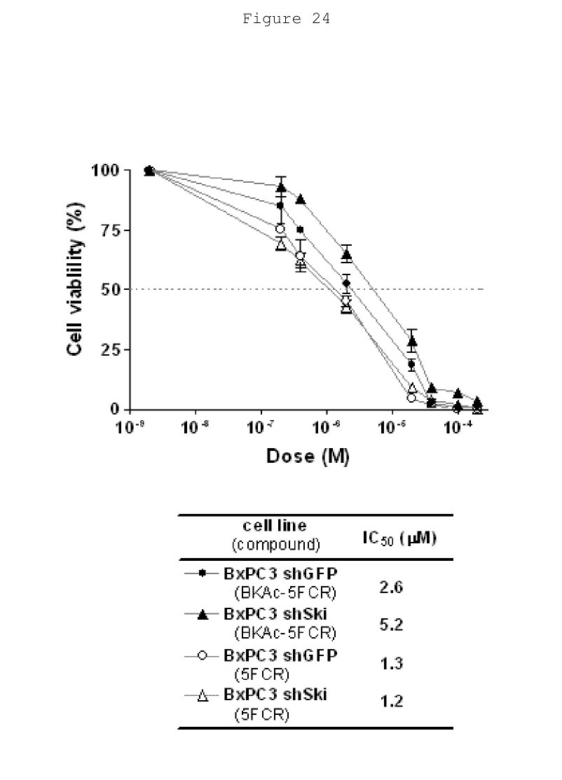

IC₅₀ values were determined for compound Boc-Lys(Ac)-5-fluorocytidine (BKAc-5FCR) and it's parental drug 5FCR in BxPC3 cells stably expressing shRNas against either GFP or Ski (shGFP and shSki). Consistent with the 50-60% difference in HDAC activity in these cells (FIGS. 4-5), a moderate but significant reduction in drug response was seen in BxPC3 shSki cells treated with BKAc-5FCR over shGFP control cells, (FIG. 24), which showed a two-fold difference in IC₅₀ values, shSki: 5.2 versus shGFP: 2.6. In contrast, no significant changes in IC₅₀ values were observed in the same cells treated with 5FCR (shSki: 1.2 versus shGFP: 1.3).

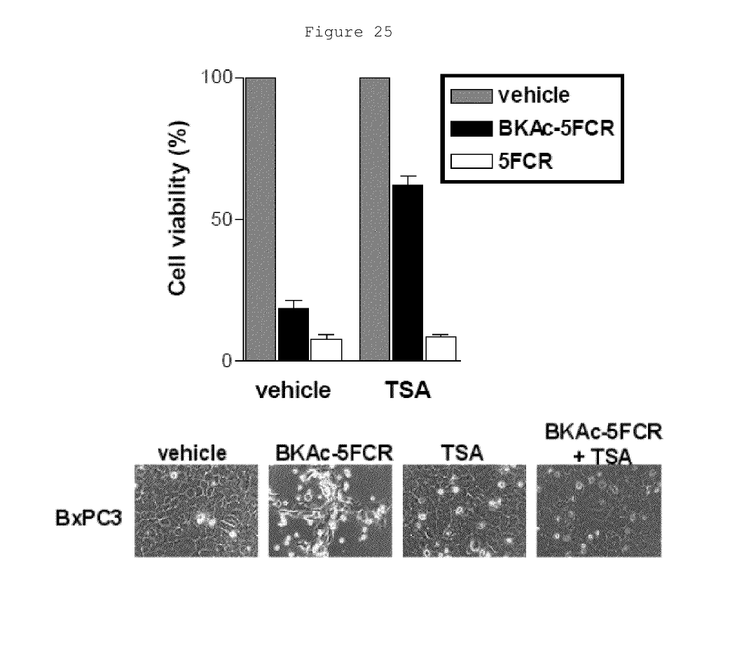

The HDAC-dependent activation of BKAc-5FCR in BXPC-3 cells was further demonstrated in the presence of HDAC inhibitor TSA where the cytotoxic effect of BKAc-5FCR was substantially compromised (FIG. 25).

### Example 6

### Additional Prodrugs

The recognition of ε-acetylated lysine residue by class I HDACs is rather insensitive to changes in sequence context. As a result, it is possible to design peptide substrates optimized with respect to the second step in the reaction, namely proteolysis. During the course of our study on characterizing HDAC activity in pancreatic cancer cell lines, we noticed that the Lys-AMC amide bond can be cleaved by endogenous proteases without adding trypsin (FIGS. 5B and 6). However this cleavage is completely inhibited by TSA treatment, indicating that TSA sensitive HDAC activity is a prerequisite.

By using pancreatic cancer cell lines such as BxPC3 and CFPac-1 cells, kinetic parameters of cleavage of the following available substrates are analyzed for cathepsin L conjugated to AMC: Z-Phe-Lys-, Boc-Phe-Lys-, Ac-Phe-Lys-, HCO-Phe-Lys-, Z-Lys-, Boc-Lys-, Ac-Lys-, HCO-Lys-, where Z (benzyloxy-carbonyl), Boc (tert-butoxycarbonyl), Ac (acetyl), and HCO (formyl) represent α-amino protecting groups.

Most of these substrates are also recognized by another lysosomal cystein protease cathepsin B, and both amino acid sequence and α-amino protecting group were reported to affect kinetic parameters of cleavage, yielding substrates that represent fast (tin/=−10 min), medium (t_{1/2}=1-3 hr), and slow (t_{1/2}=8-10 hr) cleavage reaction (Dubowchik, G. M. & Firestone, R. A. 1998). Based on the similarity in substrate recognition by cathepsin B and L, we the speed of drug activation is controlled by selecting some of the above substrates to optimize overall efficacy of the drugs. Having identified a peptide sequence, additional prodrugs are synthesized as described previously. The procedure is simple and straightforward and does not require special concerns. In fact, similar amide coupling reactions have been reported (Manfredini, S. et al. 2000; Balajthy, Z. et al. 1992).

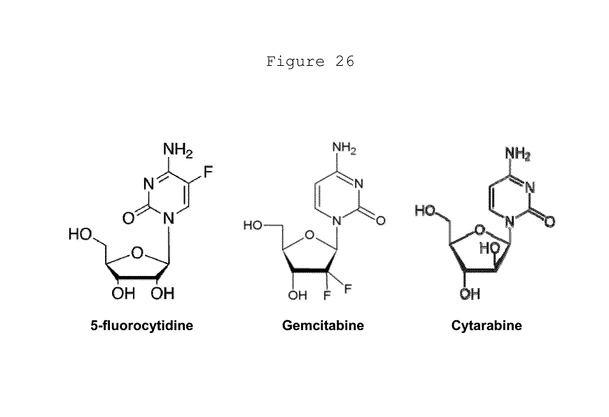

An additional aspect of the invention provides compounds with variable peptide substrates and variable parental amine-containing and non-amine containing nucleosides and deoxynucleosides including 2′-deoxy-5-fluorocytidine, 5′-deoxy-5-fluorocytidine (precursors of 5-FU), Gemicitabine, and Cytarabine (FIG. 26). Such compounds are synthesized by a similar amide coupling and are expected to function analogously to compound BKAc-5FCR and BKAc-Puro.

An additional aspect of the invention provides compounds with variable peptide substrates and variable parental amine-containing containing chemotherapeutic agents and are synthesized by a similar amide coupling. Such compounds are expected to function analogously to compound BKAc-5FCR and BKAc-Puro.

An additional aspect of the invention provides compounds with variable peptide substrates and variable non-amine containing chemotherapeutic agents and are synthesized by an amide coupling of the peptide substrate to a “Y” linker which is attached to the chemotherapeutic agent. Compounds with a variety of “Y” linkers are expected to function analogously to compound BKAc-5FCR and BKAc-Puro. The linker “Y” may be a “self-immolative” linker, which cleaves spontaneously after the carrier-linker bond, i.e. the amide bond, is broken. Example of such a “self-immolative” linker includes, nut is not limited to, a para-aminobenzyl alcohol linker (Richard, J. et al. 2008).

An additional aspect of the invention provides compounds with variable peptide substrates and variable chemotherapeutic agents that are customized to act as selective substrates for any of the specific HDACs, e.g., HDAC3.

An additional aspect of the invention provides compounds with variable peptide substrates and variable therapeutic agents that are customized to act as therapeutics for any disease that is associated with elevated levels of HDACs, proteases or both. Such diseases include, but are not limited to, neurodegenerative diseases (Chuang, D. et al. 2009), Alzheimers disease, Parkinson's disease, neuropsychiatric diseases (Fischer, A. et al. 2010), infectious disease such as HIV/AIDS (Andrew, K. T. et al. 2012), parasitic diseases (Andrew, K. T. et al. 2012) or inflammatory diseases (Halili, M. A. et al. 2009).

### Example 7

### Additional Biological Studies

Other Cell Lines

The prodrugs are evaluated by determining IC₅₀ values in other cancer cell lines. It is expected that cells which have the lowest HDAC levels, either due to improved knockdown of Ski levels or as a direct result of shRNA directed against HDAC3 (and potentially other HDACs) would be more resistant to the prodrugs. Conversely, it is expected that cells which have the highest HDAC levels would be less resistant to the prodrugs.

Monitoring of Prodrugs by HPLC

To confirm that the prodrugs are processed by HDACs and cathepsin L, commercially available purified enzymes (class I HDACs and cathepsin L are available from TEBU-BIO) are used either alone or in combination with each other, as well as assaying directly cell extracts after drug treatment. The processing of the prodrugs is monitored by HPLC (C-18 column) or TLC. In addition, hydrolysis studies are performed by measuring enzymatic and non-enzymatic stability of the prodrugs in human plasma, tissue culture medium containing 20% fetal calf serum, and defined buffer solutions with variable pH.

### Example 8

### Cell Death Induced by Boc-Lys(Ac)-Puromycin

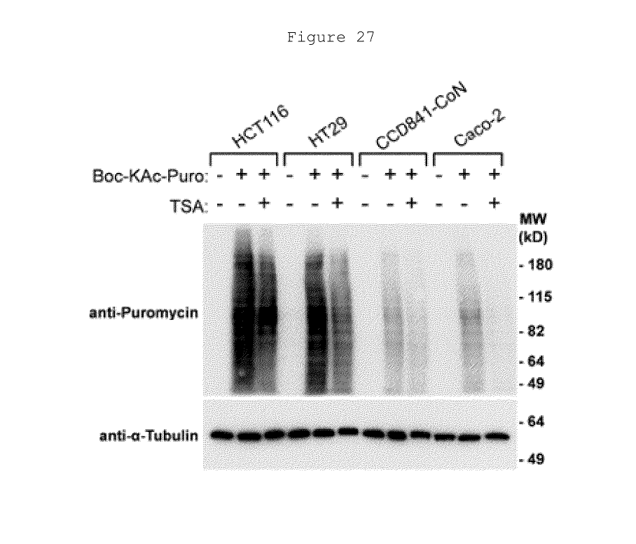

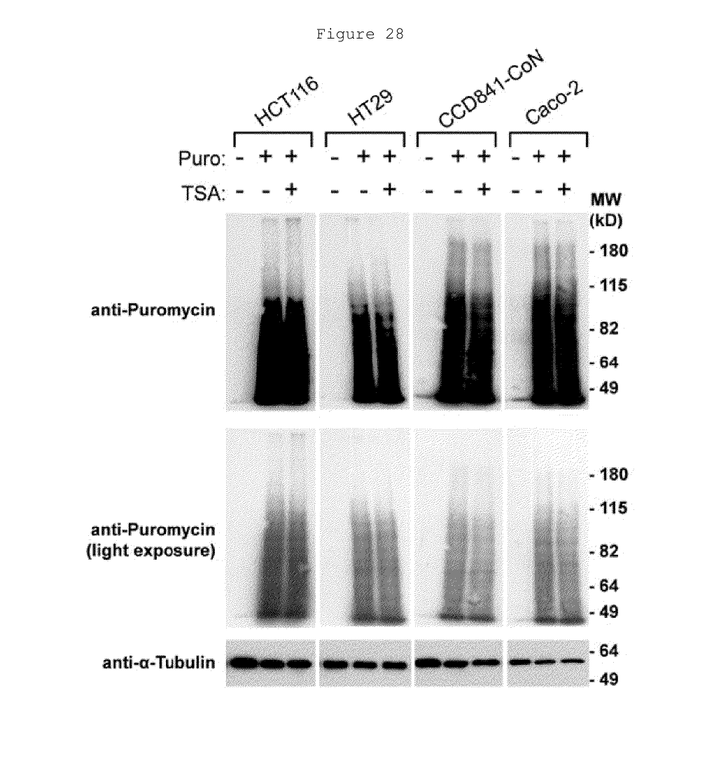

The mechanism of cell death induced by Boc-KAc-Puro was examined. As a Tyr-tRNA mimetic, Puro enters the ribosome A site and blocks peptide chain elongation by covalent incorporation into the C terminus of nascent polypeptide chains (Pestka, S. et al. 1972). In contrast, the prodrug is unable to do so since its active center amino group is masked. This allows for the monitoring of active Puro conversion in cells by immunological detection of Puro-labeled proteins using anti-Puro antibody (Schmidt, E. K. et al. 2009). To confirm that Boc-KAc-Puro is selectively converted into Puro in malignant cancer cells resulting in their death, the level of Puro incorporation was assessed by immunoblotting using cell lysates from cells treated with the agent. The intensity of Puro incorporation was substantially greater in malignant cancer cells (HCT116 and HT29) compared with normal and non-malignant cells (CCD841-CoN and Caco-2) (FIG. 27) in accordance with their sensitivity to the agent. The compromised intensity in the presence of TSA was at least partially attributable to the specific requirement of HDAC activity for the prodrug activation. When these cells were treated with parental Puro, there was no difference in terms of Puro incorporation regardless of TSA treatment (FIG. 28). This is consistent with the ability of parental Puro to kill all of these cells examined. The results indicated that the agent can be selectively converted into Puro in malignant cancer cells leading to their death by a Puro-dependent mode of action.

### Example 9

### Tumor Growth Inhibition by Boc-Lys(Ac)-Puromycin

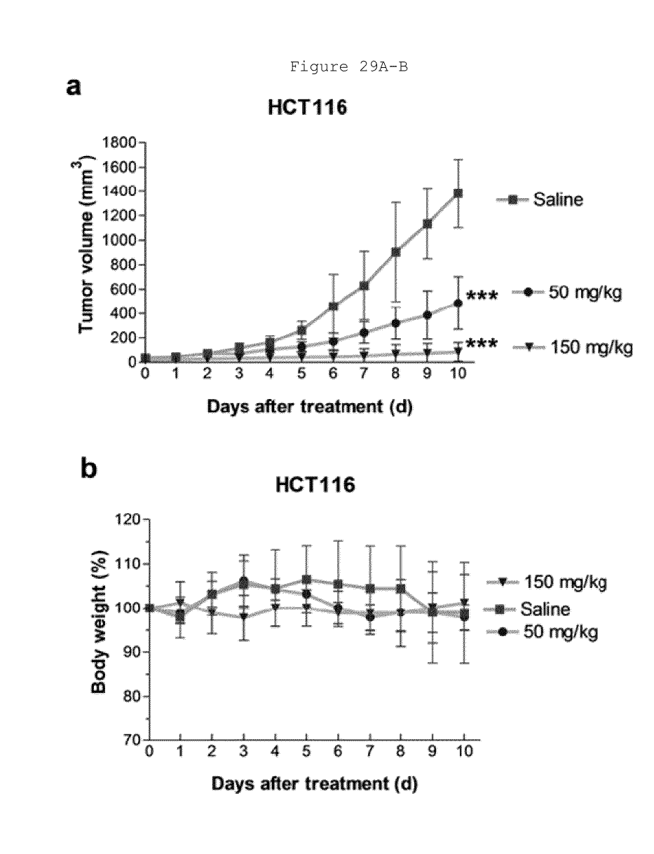

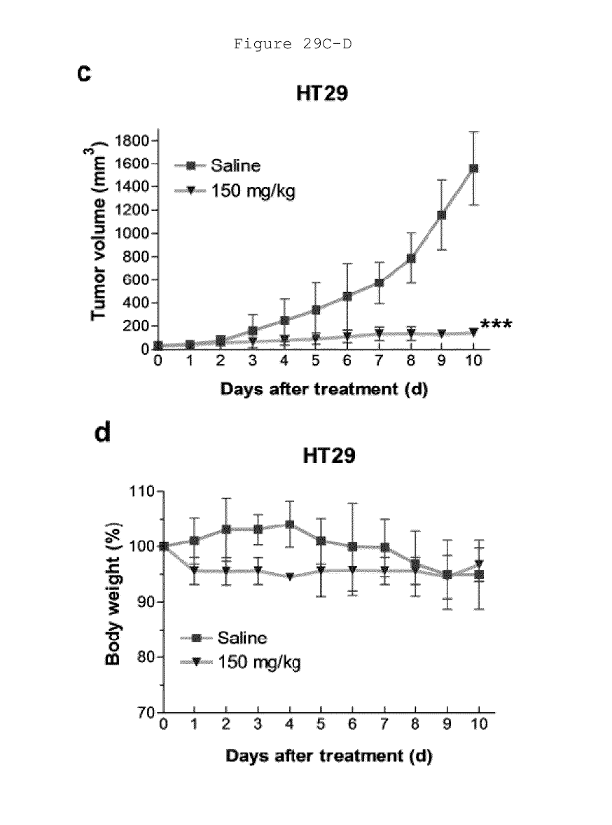

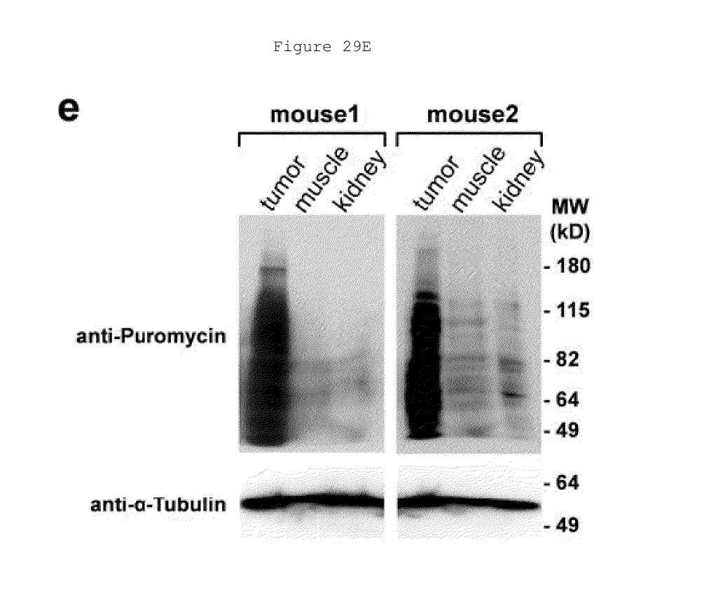

To assess in vivo anticancer efficacy of the agent, mouse xenograft models bearing human colon cancer cell lines were used (HCT116 and HT29). First, HCT116 cells were subcutaneously injected into the lower flank of mice, then dosing was initiated when small palpable tumors had developed (>3 mm in diameter). Boc-KAc-Puro was daily administered intraperitoneally at 50 and 150 mg/kg/dose for 10 d. The agent caused a dose-dependent inhibition of tumor growth (FIG. 29A). In this model, animals treated with the prodrug developed significantly smaller tumor mass in comparison to the animals treated with acidified saline control (P<0.001). Furthermore, daily intraperitoneal administration of the prodrug as high as 150 mg/kg/dose for 10 d appeared well-tolerated as judged by weight loss determination (FIG. 29B). Similar results were obtained using HT29 cells at 150 mg/kg/dose (FIG. 29C-D). These results clearly demonstrated in vivo anticancer efficacy of the prodrug Boc-KAc-Puro without severe off-target systemic toxicity.

To assess if the prodrug is selectively activated at tumor sites in vivo, levels of Puro incorporation into polypeptides in tumors and normal tissues were monitored, including muscle underneath the tumors and kidney, one of the major excretion sites for drug metabolites. Animals bearing HT29 xenograft tumor were given daily intraperitoneal administration of the prodrug at 150 mg/kg/dose for 3 d followed by preparation of tissue lysates for immunological detection of Puro-labeled proteins. The intensity of Puro incorporation was substantially greater in tumors compared with normal muscle and kidney (FIG. 29E), further supporting tumor specific activation of the agent in vivo. The results indicated that the agent can be selectively converted into Puro in tumor tissues leading to their growth inhibition by Puro-dependent mode of action.

### Example 10

### Selective Puromycin-Based Imaging Probes

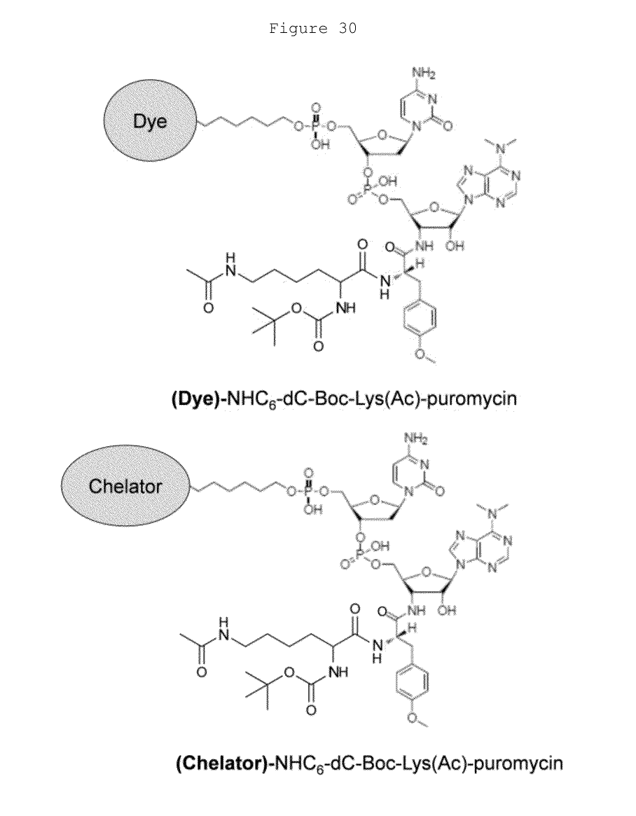

An additional aspect of the invention provides compounds with imaging agents that can be selectively removed in cancer cells but not in normal cells. The technology provides superior noninvasive cancer detection by utilizing combined enzymatic activities of histone deacetylase (HDAC) and Cathepsin L (CTSL). Masked puromycin-based imaging probes (FIG. 30) selectively label cancer cells expressing both enzymes where they are activated (releasing the amino group of the puromycin) and conjugated to proteins being synthesized. Relative to normal cells, local probe concentrations in cancer cells are retained substantially higher over longer time period. Thus, the masked puromycin derivatives enable in vivo cancer cell specific labeling, resulting in greater sensitivity and signal-noise ratio as well as safety.

An additional aspect of the invention provides compounds with variable peptide substrates and variable amine-containing and non-amine containing imaging agents.

An additional aspect of the invention provides compounds with variable peptide substrates and variable non-amine containing imaging agents and are synthesized by an amide coupling of the peptide substrate to a “Y” linker which is attached to the imaging agent. The linker “Y” may be a “self-immolative” linker, which cleaves spontaneously after the carrier-linker bond, i.e. the amide bond, is broken. Example of such a “self-immolative” linker includes, nut is not limited to, a para-aminobenzyl alcohol linker (Richard, J. et al. 2008).

An additional aspect of the invention provides compounds with variable peptide substrates and variable imaging agents that are customized to act as selective substrates for any of the specific HDACs, e.g., HDAC3.

### Example 11

### Live Single Cancer Cell Imaging Using Boc-KAc-Puro by Fluorecence Microscope and FACS

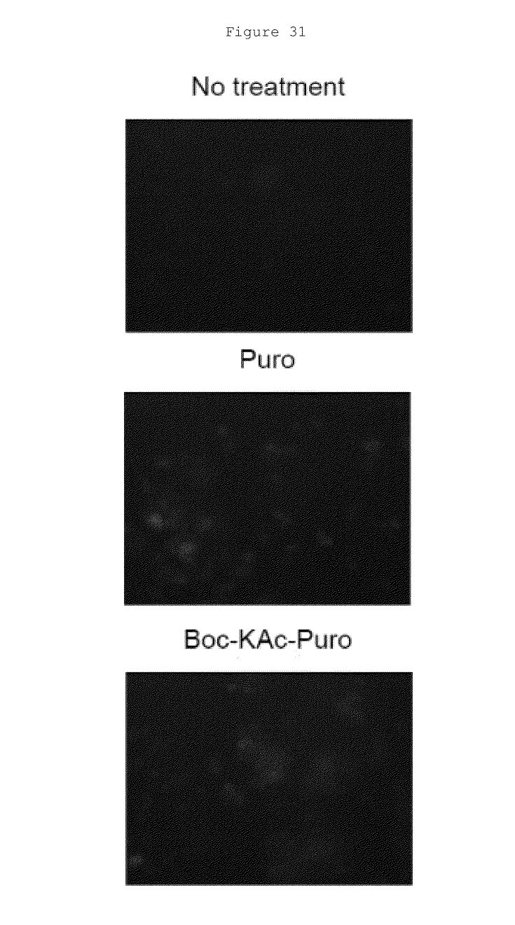

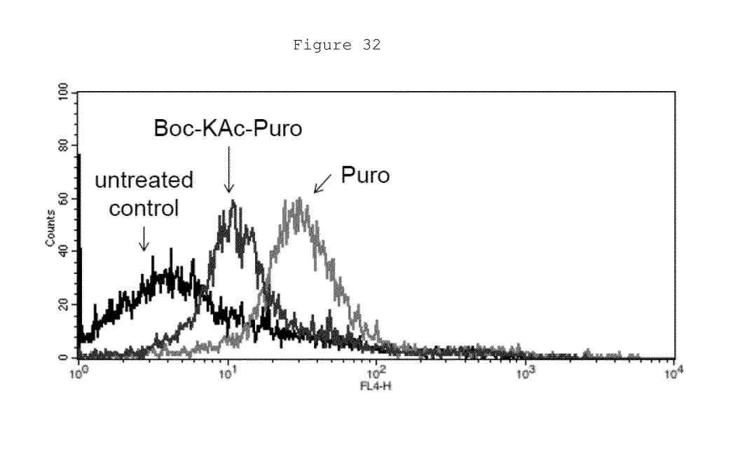

Live single cell immunodetection of Puro has been demonstrated by detecting cell surface proteins labelled with Puro (Schmidt, E. K. et al. 2009). To test if the Boc-KAc-Puro is activated in cancer cells resulting in presenting Puro on their cell surface proteins, cell surface detection of Puro by anti-puro antibody (12D10) conjugated to Alexa 647 was monitored. The Puro presentation on the cell surface proteins was confirmed by fluorescence microscopy (FIG. 31) and FACS (FIG. 32). The results demonstrated that the agent enables live cell imaging of single cancer cells. This is particularly important, because this technique allows selective imaging of live single cells by fluorecence microscopy and in heterogenous populations of cells by FACS.

### Example 12

### Single Cancer Cell Imaging in a Subject Using Boc-KAc-Puro

Boc-KAc-Puro is administered to a subject afflicted with cancer.

Puromycin is released from the Boc-KAc-Puro and accumulates at the cancer cells in the subject. An anti-puromycin antibody, which is conjugated to a detectable marker, imaging moiety or imaging agent, is administered to the subject and binds to the puromycin at the cancer cells in the subject. The cancer cells in the subject are detected using a molecular imaging device based on the location of the detectable marker, imaging moiety or imaging agent in the subject and an image of the cancer cells is obtained.

Discussion

Histone deacetylases (HDACs) are the key enzymes involved in the epigenetic regulation of histone and non-histone proteins by modulating protein structure and function through deacetylation of lysine residues (Witt, O. et al. 2009). Protein lysine acetylation is tightly regulated by HDACs and histone acetyltransferases (HATs), which influence chromatin dynamics, protein turnover and DNA damage response (Witt, O. et al. 2009; Lee, K. K. & Workman, J. L. 2007; Choudary, C. et al. 2009). Thus disregulation of these enzymes could lead to a broad spectrum of human diseases including cancer. Accumulating evidence indicate pro-proliferative and pro-survival roles of HDACs to support tumor initiation, progression and metastasis (Haberland, et al. 2009; Mariadason, J. M. 2008; Wu, M. Z. et al. 2011).

In order to develop better therapeutics to take advantage of HDACs, a new approach is needed. Instead of using HDAC inhibitors (Minucci, S. et al. 2006), intrinsically elevated HDAC activity can be taken advantage of in order to selectively deliver cytotoxicity to the tumor cells. Since certain tumor cells are reliant on their elevated levels of HDACs to survive and proliferate under stressful conditions, a chemical HDAC substrate coupled to a therapeutic agent can preferentially cause lethality in cells with high, but not low, HDAC activity. Upon deacetylation by HDAC, the HDAC substrate is, in turn, recognized as a substrate by specific intracellular proteases that cleave amide bonds, which ultimately results in release of the therapeutic agent. Although many peptide-based prodrugs activated by tumor-associated proteases have been developed (Choi, K. Y. et al. 2012), stability and nonspecific activation of parental drugs due to ubiquitous proteases are key factors limiting their clinical efficacy. By introducing an ε-acetylated lysine as the first requirement, the results disclosed herein show that the resulting prodrug can be well-protected from proteolytic cleavage until it is deacetylated by the intracellular HDAC, and thus enable highly selective drug activation in tumor tissues.

This approach allows for the targeting of two independent enzymes aberrantly activated in tumor cells, providing better selectivity. In addition, because HDAC activity is undetectable in plasma, and the amide bond between HDAC substrate and therapeutic agent is hardly cleaved by ubiquitous proteases in cytoplasm or plasma, this approach also minimizes the known drawbacks of peptide-based prodrugs.

Described herein is a new approach for selective cancer therapy by targeting increased histone deacetylase (HDAC) and increased protease activity in certain cells. Therefore, the prodrug itself or the therapeutic agent X should not be an HDACi or protease inhibitor. This approach is promising strategy for the next generation of selective anticancer drugs.

In order to develop better and more selective therapeutic approaches targeting HDACs, we took advantage of certain tumor cells that are more reliant on their elevated level of HDAC and CTSL to survive and proliferate under stressful conditions. The elevated level of HDACs in tumor cells result in cells that are highly sensitive to HDACi. Thus, instead of inhibiting HDACs, novel activity based agents were designed that selectively eliminate cancer cells by taking advantage of their elevated HDAC activity.

Although nucleosides such as gemicitabine (Heunemann, V. et al. 2011) and 5-FU (5-fluorouracil) (Lamont, E. B. et al. 1999) are current standard therapeutic regimens for pancreatic tumors, their efficacy is far from an ideal treatment for this devastating disease. Thus, there is an urgent need to develop drugs that are more effective than these conventional drugs. As explained herein, elevated HDAC activity, which is mediated by Ski oncoprotein in pancreatic ductal adenocarcinoma (PDA) can provide a therapeutic target.

Selective prodrugs incorporating nucleoside parent agents were designed and synthesized. These prodrugs were synthesized by coupling “nonselective” nucleoside antimetabolite analogs such as 5-fluorocytidine and puromycin to HDAC substrates to produce prodrugs BKAc-5FCR and BKAc-puro, respectively. The prodrugs were activated by HDACs and proteases, which resulted in selective delivery of the parent drug to the cancer cells.

Since a free N4-amino group of the parental drug of BKAc-5FCR is required for cytotoxicity, this group was protected by conjugating a peptide HDAC substrate containing ε-acetylated lysine residue through amide coupling. In this way, the activation of prodrugs was executed by a HDAC-dependent two-step enzymatic reaction. In the first step catalyzed by HDACs, acetate is released from ε-acetylated lysine. In the next step, the deacetylated lysine is recognized as substrate by specific intracellular proteases that cleave carboxyl terminal lysil amide bonds, releasing active drugs with the free N4-amino group. This approach targeted two independent key enzymes aberrantly activated in tumor cells, providing better selectivity. In addition, because HDAC activity is undetectable in plasma, and the amide bond between ε-acetylated lysine and parental drugs is hardly cleaved by proteases, our approach minimizes the known drawbacks of peptide-based prodrugs such as nonspecific activation by ubiquitous proteases in cytoplasm or plasma.

Although peptide-based nucleoside analogs activated by tumor associated proteases were developed previously (Carl, P. L. et al. 1980; Dubowchik, G. M. et al. 1998; Dubowchik, G. M. & Firestone, R. A. 1998; Balajthy, K. et al. 1992), we identified that nonspecific release of parental drugs by ubiquitous proteases in cell cytoplasm or plasma was a major drawback. To address such drawbacks and further improve selectivity in tumor cells, we designed prodrugs that were activated by two independent enzymatic reactions: HDACs and tumor associated proteases. By targeting these key enzymes that act in epigenetic and proteolytic pathways in tumors, we have developed prodrugs for selective cancer chemotherapy.

The prodrugs that are synthesized may have some basal level of cytotoxicity before they are activated by HDAC3. One way to determine the IC₅₀ values of the uncleaved forms of the prodrugs would be to use HDAC inhibitors both in vitro and in vivo. In preliminary in vitro experiments, TSA was used to inhibit HDACs in the cells, unfortunately due to the cytotoxic effect of TSA at 1 μM over the 72 hr time period, ICS values were not obtained. To overcome this difficulty, TSA concentration is reduced by titrating to levels (10-50 nM) that should maintain maximal HDAC inhibition and minimal cytotoxicity. HDAC1, 2, 3, and 6, among others, are potential candidate enzymes capable of prodrug activation and they are all known to be TSA sensitive (Bradner, J. E. et al. 2010), hence this range of TSA concentration should sufficiently inhibit all of these enzymes. Should it prove impossible to find a range of TSA that allows 72 hr cell toxicity assays, the use of other HDAC inhibitors is explored by routine experimentation. For example, among available HDAC inhibitors, SAHA (Vorinostat), a FDA approved pharmaceutical HDAC inhibitor (Bradner, J. E. et al. 2010), is known to inhibit all of these HDACs with much lower cytotoxicity than TSA. Thus, SAHA is an alternative HDAC inhibitor for the prodrug evaluation as well. By comparing the prodrug ICo values for cytotoxicity under conditions of HDAC inhibition with those values where HDACs are active we are able to provide data on the HDAC dependence of our new class of prodrugs.

Although the evaluation method using AMC-conjugated peptide substrates is rapid, highly sensitive and accurate, HPLC (high-performance liquid chromatography) or TLC (thin-layer chromatography) methods are used to directly evaluate the kinetics of the activation of the substrates conjugated to the parental drugs. As already noted in FIG. 5 and as depicted in FIG. 23, the peptide sequence can influence the ability of the compounds to be recognized by HDAC and activated by the cellular proteases. Therefore different peptide sequences are used which result in compounds with more specificity. It is also possible that because different tumor cells express different proteases or different levels of tumor-associated proteases there may be some cell-specific aspects to the prodrug activation. Therefore, an expanded cohort of PDA cell lines is evaluated.

Disclosed herein is the design, synthesis and characterization of Boc-KAc-Puro, a novel prodrug targeting increased HDAC and CTSL activities in malignant tumors. Anticancer efficacy of the agent is evidenced by its ability to inhibit tumor growth in vivo without severe adverse effects. Targeting elevated HDAC and CTSL activities in malignant cancer cells has been established as a strategy for anticancer drug development. Notably, this approach is advantageous because the simple small molecule masking group could be readily applied to many other cytotoxic agents to confer selectivity that substantially improves their therapeutic index. A potent and selective anticancer agent has been developed from a mere general cytotoxic drug Puro.

Both amino acid sequence and α-amino protecting group were reported to affect kinetic parameters of peptide-based prodrug activation by cathepsin B30. Based on the similarity in substrate recognition by cathepsin B and L, it is possible to optimize the speed of drug activation for overall efficacy. Additional embodiments could be achieved by the modification of the masking group Boc-Lys(Ac). The anticancer efficacy may be improved by introducing different peptide substrates that speed up drug activation (Wegener, D. et al. 2003).

The strategy disclose herein is applied to many chemotherapeutic drugs currently used in the clinics aiming to improve their anticancer efficacy and safety. For example, amide coupling-based prodrugs of gemcitabine and cytarabine were developed by masking their amino group (Bender, D. M. et al. 2009; Cheon, E. P. et al. 2006). Thus introduction of the Boc-Lys(Ac) group to their amino group is feasible for evaluation of their improved efficacy. In addition, when coupled with imaging probes, the masking group could be viable tools for bioimaging and non-invasive diagnosis of cancer cells. Imaging probes including Puro-based radiopharmaceuticals (Eigner, S. et al. 2013) and fluorescent agents (Starck, S. R. et al. 2004) could be modified to highly specific and safe probes for detection of cancer cells in the body.

A new cancer cell specific labeling technique was developed by taking advantage of a puromycin-based strategy to monitor global protein synthesis in vivo. As a Tyr-tRNA mimetic, puromycin enters the ribosome A site and blocks peptide chain elongation by covalent incorporation into the C terminus of nascent polypeptide chains (Miyamoto-Sato et al 2000; Pestka et al 1972). This puromycin-based strategy has been developed as a nonradioactive technique to monitor global protein synthesis in live cells as an alternative to traditional metabolic labeling methods using radiolabeled amino acids (Schmidt et al 2009). Puromycin derivatives including radiopharmaceuticals and fluorescent agents have also been developed to monitor global protein synthesis in vivo (Eigner et al 2013, Liu et al 2012; Starck et al 2004). However, they lack specificity to cancer cells thus cannot be used to detect cancer for imaging or diagnostic purposes in their current forms. In addition, these applications are very limited due to the cytotoxic nature of puromycin.

To create highly specific and safe probes for cancer cell detection in the body, a masking group that can be selectively removed in cancer cells but not in normal cells is introduced into puromycin. The technology provides superior noninvasive cancer detection by utilizing combined enzymatic activities of histone deacetylase (HDAC) and Cathepsin L (CTSL) that were proved to be specific markers of malignant cancer cells. By coupling the masking group that requires both HDAC and CTSL to be removed, masked puromycin-based imaging probes selectively label cancer cells expressing both enzymes where they will be activated and conjugated to proteins being synthesized. In this way, relative to normal cells, local probe concentrations in cancer cells can be retained substantially higher over longer time period. Thus, the masked puromycin derivatives enable in vivo cancer cell specific labeling, resulting in greater sensitivity and signal-noise ratio as well as safety.

The highly selective in vivo cancer labeling technology is an innovative strategy for the next generation of cancer detection, and it can be readily applied to existing imaging technologies including MRI and PET. Successful development of such agents has the potential to impact early cancer detection and monitoring of tumor response to therapy.

The observation that malignant cancer cells exhibit high levels of HDAC and CTSL activities has important implications for the potential use of their combined enzymatic activities as a selective modality for delivering therapeutics to their targets. By utilizing a small molecule masking group, a prototypic agent was specifically activated by these enzymes in cancer cells. Such agents have the potential to improve clinical outcomes as well as quality of life for the patients. Eradication of tumor cells while minimizing damage to healthy cells is a primary goal of cancer therapy. A new prodrug strategy has been developed herein for selective cancer therapy that utilizes increased histone deacetylase (HDAC) and tumor-associated protease activities produced in malignant cancer cells. By coupling an acetylated lysine, a requirement for HDAC with an endogenous protease cathepsin L (CTSL), a masked cytotoxic agent puromycin selectively causes lethality in human cancer cell lines with high HDAC and CTSL activities. In vivo studies confirmed tumor growth inhibition due to selective drug activation in the prodrug treated mice bearing human colon cancer xenografts.

Further, puromycin can be presented on the cell surface proteins (Schmidt et al 2009). This is particularly important, because it can be utilized for live cells both in vitro and in vivo. Thus by taking advantage of fluorescent dye conjugated anti-puromycin antibodies, this technique allows selective imaging of live single cells by fluorescence microscopy and in heterogenous populations of cells by fluorescence activated cell sorting (FACS) as well as in vivo imaging of cancer cells by injecting the antibodies. Accordingly, cancer cells are detected in a subject by administering to the subject a puromycin containing compound of the present invention and an anti-puromycin antibody.

### REFERENCES

Andrews, K. T. HDAC inhibitors in parasitic diseases. Immunol Cell Biol. 90, 1, 66-77 (2012).
Balajthy, Z., Aradi, J., Kiss, I. T. & Elodi, P. Synthesis and functional evaluation of a peptide derivative of 1-beta-D-arabinofuranosylcytosine. J Med Chem 35, 3344-3349 (1992).
Bhaskara, S. et al. HDAC3 is essential for the maintenance of chromatin structure and genome stability. Cancer Cell 18, 436-447 (2010).
Bolden, J. E., Peart, M. J. & Johnstone, R. W. Anticancer activities of histone deacetylase inhibitors. Nat Rev Drug Discov 5, 769-784 (2006).
Bonfils, C. et al. Evaluation of the pharmacodynamic effects of MGCD0103 from preclinical models to human using a novel HDAC enzyme assay. Clin Cancer Res 14, 3441-3449 (2008).
Bradner, J. E. et al. Chemical phylogenetics of histone deacetylases. Nat Chem Biol 6, 238-243 (2010).
Cao, Z. A. et al. CRA-026440: a potent, broad-spectrum, hydroxamic histone deacetylase inhibitor with antiproliferative and antiangiogenic activity in vitro and in vivo. Molecular cancer therapeutics 5, 1693-1701 (2006).
Carl, P. L., Chakravarty, P. K., Katzenellenbogen, J. A. & Weber, M. J. Protease-activated “prodrugs” for cancer chemotherapy. Proc Natl Acad Sci USA 77, 2224-2228 (1980).
Cheon, E. P. et al. Enhanced cellular uptake of Ara-C via a peptidomimetic prodrug, L-valyl-ara-C in Caco-2 cells. The Journal of pharmacy and pharmacology 58, 927-932 (2006).
Choi, K. Y., Swierczewska, M., Lee, S. & Chen, X. Protease-activated drug development. Theranostics 2, 156-178 (2012).
Choudhary, C. et al. Lysine acetylation targets protein complexes and co-regulates major cellular functions. Science 325, 834-840 (2009).
Chuang, D. et al. Multiple roles of HDAC inhibition in neurodegenerative conditions. Trends Neurosci. 32, 591-601 (2009).
Collette, J. et al. Enhanced cathepsin L expression is mediated by different Ras effector pathways in fibroblasts and epithelial cells. Int J Cancer 112, 190-199 (2004).
Denhardt, D. T. et al. Cysteine proteinase cathepsin L expression correlates closely with the metastatic potential of H-ras-transformed murine fibroblasts. Oncogene 2, 55-59 (1987).
Dubowchik, G. M. & Firestone, R. A. Cathepsin B-sensitive dipeptide prodrugs. 1. A model study of structural requirements for efficient release of doxorubicin. Bioorg Med Chem Lett 8, 3341-3346 (1998).
Dubowchik, G. M. et al. Cathepsin B-sensitive dipeptide prodrugs. 2. Models of anticancer drugs paclitaxel (Taxol), mitomycin C and doxorubicin. Bioorg Med Chem Lett 8, 3347-3352 (1998).
Eigner, S., et al. Imaging of protein synthesis: in vitro and in vivo evaluation of (44)Sc-DOTA-puromycin. Molecular imaging and biology: MIB: the official publication of the Academy of Molecular Imaging 15, 79-86 (2013).
Fischer, A. Targeting the correct HDAC(s) to treat cognitive disorders. Trends Pharmacol Sci. 31, 12 (2010).
Frangioni, J. V. (2008) New Technologies for Human Cancer Imaging. Journal of Clinical Oncology 26, 4012-4021.
Gallagher B M, et al. (1977). Radiopharmaceuticals XXVII. 18F-labeled 2-deoxy-2-fluoro-d-glucose as a radiopharmaceutical for measuring regional myocardial glucose metabolism in vivo: tissue distribution and imaging studies in animals. Journal of nuclear medicine: official publication, Society of Nuclear Medicine 18: 990-996.
Gonzalez-Suarez, I. et al. A new pathway that regulates 53BP1 stability implicates cathepsin L and vitamin D in DNA repair. EMBO J 30, 3383-3396 (2011).
Goulet, B., et al. A cathepsin L isoform that is devoid of a signal peptide localizes to the nucleus in S phase and processes the CDP/Cux transcription factor. Molecular cell 14, 207-219 (2004).
Goulet, B., et al. Increased expression and activity of nuclear cathepsin L in cancer cells suggests a novel mechanism of cell transformation. Molecular cancer research: MCR 5, 899-907 (2007).
Grotsky, D. A., et al. BRCA1 loss activates cathepsin L-mediated degradation of 53BP1 in breast cancer cells. J Cell Biol 200, 187-202 (2013).
Haberland, M. et al. Genetic dissection of histone deacetylase requirement in tumor cells. Proc Natl Acad Sci USA 106, 7751-7755 (2009).
Halili, M. A. et al. Histone Deacetylase Inhibitors In Inflammatory Disease. Curr Top Med Chem. 9, 309-319 (2009).
Heinemann, V. Gemcitabine: progress in the treatment of pancreatic cancer. Oncology 60, 8-18 (2001).
Jedeszko, C. & Sloane, B. F. Cysteine cathepsins in human cancer. Biol Chem 385, 1017-1027 (2004).
Joseph, L. J., Chang, L. C., Stamenkovich, D. & Sukhatme, V. P. Complete nucleotide and deduced amino acid sequences of human and murine preprocathepsin L. An abundant transcript induced by transformation of fibroblasts. J Clin Invest 81, 1621-1629 (1988).
Joyce, J. A. et al. Cathepsin cysteine proteases are effectors of invasive growth and angiogenesis during multistage tumorigenesis. Cancer Cell 5, 443-453 (2004).
Kano, M. R. et al. Improvement of cancer-targeting therapy, using nanocarriers for intractable solid tumors by inhibition of TGF-beta signaling. Proc Natl Acad Sci USA 104, 3460-3465 (2007).
Lankelma, J. M. et al. Cathepsin L, target in cancer treatment? Life Sci 86, 225-233 (2010).
Lee, J. H., Choy, M. L., Ngo, L., Foster, S. S. & Marks, P. A. Histone deacetylase inhibitor induces DNA damage, which normal but not transformed cells can repair. Proc Natl Acad Sci USA 107, 14639-14644 (2010).
Lee, K. K. & Workman, J. L. Histone acetyltransferase complexes: one size doesn't fit all. Nat Rev Mol Cell Biol 8, 284-295 (2007).
Liu J, Xu Y, Stoleru D, Salic A (2012). Imaging protein synthesis in cells and tissues with an alkyne analog of puromycin. Proc Natl Acad Sci USA 109: 413-418.
Manfredini, S. et al. Peptide T-araC conjugates: solid-phase synthesis and biological activity of N4-(acylpeptidyl)-araC. Bioorg Med Chem 8, 539-547 (2000).
Marks, P. A. & Xu, W. S. Histone deacetylase inhibitors: Potential in cancer therapy. J Cell Biochem 107, 600-608 (2009).
Mariadason, J. M., Velcich, A., Wilson, A. J., Augenlicht, L. H. & Gibson, P. R. Resistance to butyrate-induced cell differentiation and apoptosis during spontaneous Caco-2 cell differentiation. Gastroenterology 120, 889-899 (2001).
Mariadason, J. M. HDACs and HDAC inhibitors in colon cancer. Epigenetics 3, 28-37 (2008).
Minucci, S. & Pelicci, P. G. Histone deacetylase inhibitors and the promise of epigenetic (and more) treatments for cancer. Nat Rev Cancer 6, 38-51 (2006).
Miyamoto-Sato E, Nemoto N, Kobayashi K, Yanagawa H (2000). Specific bonding of puromycin to full-length protein at the C-terminus. Nucleic Acids Res 28: 1176-1182.
Niedergethmann, M. et al. Prognostic impact of cysteine proteases cathepsin B and cathepsin L in pancreatic adenocarcinoma. Pancreas 29, 204-211 (2004).
Oft, M., et al. TGF-beta1 and Ha-Ras collaborate in modulating the phenotypic plasticity and invasiveness of epithelial tumor cells. Genes Dev 10, 2462-2477 (1996).
O'Toole, J. M. et al. Therapeutic implications of a human neutralizing antibody to the macrophage-stimulating protein receptor tyrosine kinase (RON), a c-MET family member. Cancer Res 66, 9162-9170 (2006).
Overholser, J. P., Prewett, M. C., Hooper, A. T., Waksal, H. W. & Hicklin, D. J. Epidermal growth factor receptor blockade by antibody IMC-C225 inhibits growth of a human pancreatic carcinoma xenograft in nude mice. Cancer 89, 74-82 (2000).
Pacheco, F. J. et al. Involvement of lysosomal cathepsins in the cleavage of DNA topoisomerase I during necrotic cell death. Arthritis Rheum 52, 2133-2145 (2005).
Pestka, S., Rosenfeld, H., Harris, R. & Hintikka, H. Studies on transfer ribonucleic acid-ribosome complexes. XXI. Effect of antibiotics on peptidyl-puromycin synthesis by mammalian polyribosomes. J Biol Chem 247, 6895-6900 (1972).
Richard, J. et al. Latent Fluorophores Based on a Self-Immolative Linker Strategy and Suitable for Protease Sensing. Bioconjugate Chem 19, 1707-1718 (2008).
Schmidt, E. K., Clavarino, G., Ceppi, M. & Pierre, P. SUnSET, a nonradioactive method to monitor protein synthesis. Nature methods 6, 275-277 (2009).
Starck, S. R., Green, H. M., Alberola-Ila, J. & Roberts, R. W. A general approach to detect protein expression in vivo using fluorescent puromycin conjugates. Chem Biol 11, 999-1008 (2004).
Tian, Y., Bova, G. S. & Zhang, H. Quantitative glycoproteomic analysis of optimal cutting temperature-embedded frozen tissues identifying glycoproteins associated with aggressive prostate cancer. Analytical chemistry 83, 7013-7019 (2011).
Vara, J. A., Portela, A., Ortin, J. & Jimenez, A. Expression in mammalian cells of a gene from Streptomyces alboniger conferring puromycin resistance. Nucleic Acids Res 14, 4617-4624 (1986).
Vecsey-Semjen, B., et al. Novel colon cancer cell lines leading to better understanding of the diversity of respective primary cancers. Oncogene 21, 4646-4662 (2002).
von Burstin, J. et al. Highly sensitive detection of early-stage pancreatic cancer by multimodal near-infrared molecular imaging in living mice. Int J Cancer 123, 2138-2147 (2008).
Wagner, J. M., Hackanson, B., Lubbert, M. & Jung, M. Histone deacetylase (HDAC) inhibitors in recent clinical trials for cancer therapy. Clin Epigenetics 1, 117-136 (2010).
Wegener, D., Wirsching, F., Riester, D. & Schwienhorst, A. A fluorogenic histone deacetylase assay well suited for high-throughput activity screening. Chem Biol 10, 61-68 (2003).
Weissleder, R., Tung, C. H., Mahmood, U. & Bogdanov, A., Jr. In vivo imaging of tumors with protease-activated near-infrared fluorescent probes. Nat Biotechnol 17, 375-378 (1999).
Witt, O., Deubzer, H. E., Milde, T. & Oehme, I. HDAC family: What are the cancer relevant targets?Cancer Lett 277, 8-21 (2009).
Wu, M. Z. et al. Interplay between HDAC3 and WDR5 is essential for hypoxia-induced epithelial-mesenchymal transition. Mol Cell 43, 811-822 (2011).
Yoshida, M., Kijima, M., Akita, M. & Beppu, T. Potent and specific inhibition of mammalian histone deacetylase both in vivo and in vitro by trichostatin A. J Biol Chem 265, 17174-17179 (1990).

1. A compound having the structure: 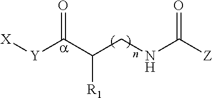
wherein
X is an imaging agent containing at least one amine nitrogen;
Y is a chemical linker,
wherein Y is present or absent, and when present Y is a chemical linker containing at least one amine nitrogen or Y is a para-aminobenzyl alcohol linker;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆alkenyl, C₂₋₆ alkynyl, aryl, or heteroaryl,
wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
wherein an amine of the amino acid or oligopeptide is substituted or unsubstituted; and
n is an integer from 0 to 6;
wherein
when Y is absent, an amine nitrogen on the imaging agent covalently bonds directly to carbon α, or
when Y is present and is a chemical linker containing at least one amine nitrogen, an amine nitrogen on the linker covalently bonds directly to carbon α, or
when Y is present and is a para-aminobenzyl alcohol linker, the nitrogen on the linker Y bonds directly to carbon α and the oxygen on the linker Y connects to the imaging agent X, or the oxygen on the linker Y bonds directly to carbon α and the nitrogen on the linker Y connects to the imaging agent X through an amide bond;
or a diastereomer, enantiomer or pharmaceutically acceptable salt of the compound.

2. The compound of claim 1 having the structure 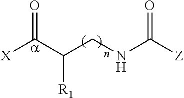
wherein
X is an imaging agent containing at least one amine nitrogen and the amine nitrogen on the imaging agent covalently bonds directly to carbon α;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
wherein an amine of the amino acid or oligopeptide is substituted or unsubstituted; and
n is an integer from 0 to 6;
or a diastereomer, enantiomer or pharmaceutically acceptable salt of the compound.

3. The compound of claim 2 having the structure: 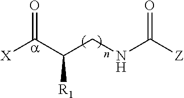
wherein
X is an imaging agent containing at least one amine nitrogen and the amine nitrogen on the imaging agent covalently bonds directly to carbon α;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
wherein an amine of the amino acid or oligopeptide is substituted or unsubstituted; and
n is an integer from 0 to 6;
or a diastereomer, enantiomer or pharmaceutically acceptable salt of the compound.

4. The compound of claim 2 having the structure: 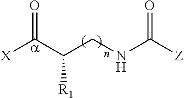
wherein
X is a an imaging agent containing at least one amine nitrogen and the amine nitrogen on the imaging agent covalently bonds directly to carbon α;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
wherein an amine of the amino acid or oligopeptide is substituted or unsubstituted; and
n is an integer from 0 to 6;
or a diastereomer, enantiomer or pharmaceutically acceptable salt of the compound.

5. (canceled)

6. A compound having the structure: 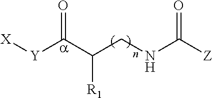
wherein
X is an imaging agent;
Y is a chemical linker;
wherein Y is present or absent, and when present,
Y is a chemical linker containing at least one amine nitrogen,
wherein the amine nitrogen on the linker covalently bonds directly to carbon α, or
Y is a para-aminobenzyl alcohol linker,
wherein the nitrogen on the linker Y connects directly to carbon α and the oxygen on the linker Y connects to the imaging agent X, or the oxygen on the linker Y connects directly to carbon α and the nitrogen on the linker Y connects to the imaging agent X through an amide bond;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
wherein the amine of the amino acid or oligopeptide is substituted or unsubstituted; and
n is an integer from 0 to 6;
or a diastereomer, enantiomer or pharmaceutically acceptable salt of the compound.

7. The compound of claim 6 having the structure: 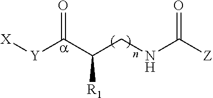
wherein
X is an imaging agent;
Y is a chemical linker;
wherein Y is present or absent, and when present,
Y is a chemical linker containing at least one amine nitrogen,
wherein the amine nitrogen on the linker covalently bonds directly to carbon α, or
Y is a para-aminobenzyl alcohol linker,
wherein the nitrogen on the linker Y connects directly to carbon α and the oxygen on the linker Y connects to the imaging agent X, or the oxygen on the linker Y connects directly to carbon α and the nitrogen on the linker Y connects to the imaging agent X through an amide bond;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
wherein the amine of the amino acid or oligopeptide is substituted or unsubstituted; and
n is an integer from 0 to 6;
or a diastereomer, enantiomer or pharmaceutically acceptable salt of the compound.

8. The compound of claim 6 having the structure: 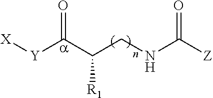
wherein
X is an imaging agent;
Y is a chemical linker;
wherein Y is present or absent, and when present,
Y is a chemical linker containing at least one amine nitrogen,
wherein the amine nitrogen on the linker covalently bonds directly to carbon α, or
Y is a para-aminobenzyl alcohol linker,
wherein the nitrogen on the linker Y connects directly to carbon α and the oxygen on the linker Y connects to the imaging agent X, or the oxygen on the linker Y connects directly to carbon α and the nitrogen on the linker Y connects to the imaging agent X through an amide bond;
Z is CH₃ or CF₃;
R₁ is —H, —NR₂R₃, —NH—C(═O)—R₄, —NH—C(═O)—OR₄, —CH₂—C(═O)—NR₅R₆, —OR₇, —CO₂R₇, C₁₋₆ alkyl, C₂₋₆ alkenyl, C₂₋₆ alkynyl, aryl, or heteroaryl,
wherein R₂, R₃, R₄, R₅, R₆ and R₇ are each, independently, —H, C₁₋₆ alkyl, C₂₋₆ alkenyl, C₂₋₆ alkynyl, heteroalkyl, cycloalkyl, heterocyclyl, aryl, alkylaryl, heteroaryl, alkylheteroaryl, an amino acid or an oligopeptide;
wherein the amine of the amino acid or oligopeptide is substituted or unsubstituted; and
n is an integer from 0 to 6;
or a diastereomer, enantiomer or pharmaceutically acceptable salt of the compound.

9.-25. (canceled)

26. The compound of claim 6, wherein the imaging agent X comprises at least one imaging moiety Q.

27. (canceled)

28. The compound of claim 26, wherein the imaging agent X is puromycin, wherein at least one ¹H in the puromycin is replaced with ³H, or at least one ¹²C in the puromycin is replaced with ¹¹C, or at least one ¹⁴N in the puromycin is replaced with ¹³N, or at least one ¹⁶O in the puromycin is replaced with ¹⁵O.

29. The compound of claim 28 having the structure:

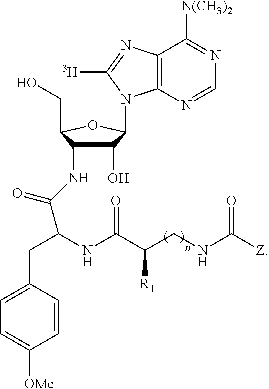

30. The compound of any one of claim 26 having the structure: 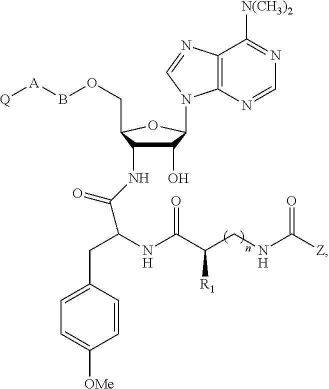
wherein
Q is an imaging moiety;
A is present or absent and when present is a alkyl linker; and
B is present or absent and when present is a nucleoside linker or nucleotide linker.

31. The compound of claim 26 having the structure: 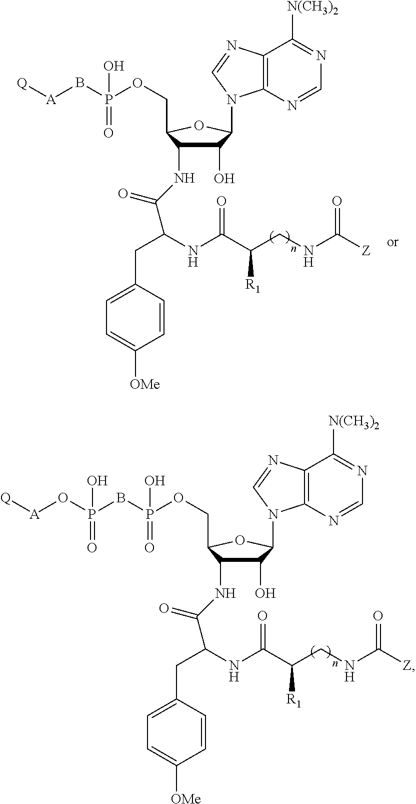
wherein
Q is an imaging moiety;
A is present or absent and when present is a alkyl linker; and
B is present or absent and when present is a nucleoside linker or nucleotide linker.

32.-33. (canceled)

34. The compound of claim 30, wherein Q is an MRI contrast imaging moiety, an optical imaging moiety, or a PET imaging moiety.

35.-53. (canceled)

54. The compound of claim 34 having the structure: 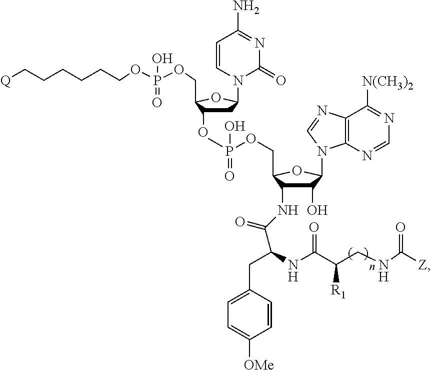
wherein Q is an ester or amide derivative of DOTA, NOTA, DTPA, TETA, CB-TE2A or CB-DO2A which is coordinated to a metal M, wherein the M is Gd³⁺, Fe³⁺, In³⁺, Mn²⁺, ^{99m}Tc, ⁹⁵Tc, ¹¹¹In, ⁶²Cu, ⁶⁴Cu, ⁴⁴Sc, ⁶⁷Ga, or ⁶⁸Ga, or Q is an ester or amide derivative of 6-FAM or Cy5.

55. The compound of claim 30 having the structure:

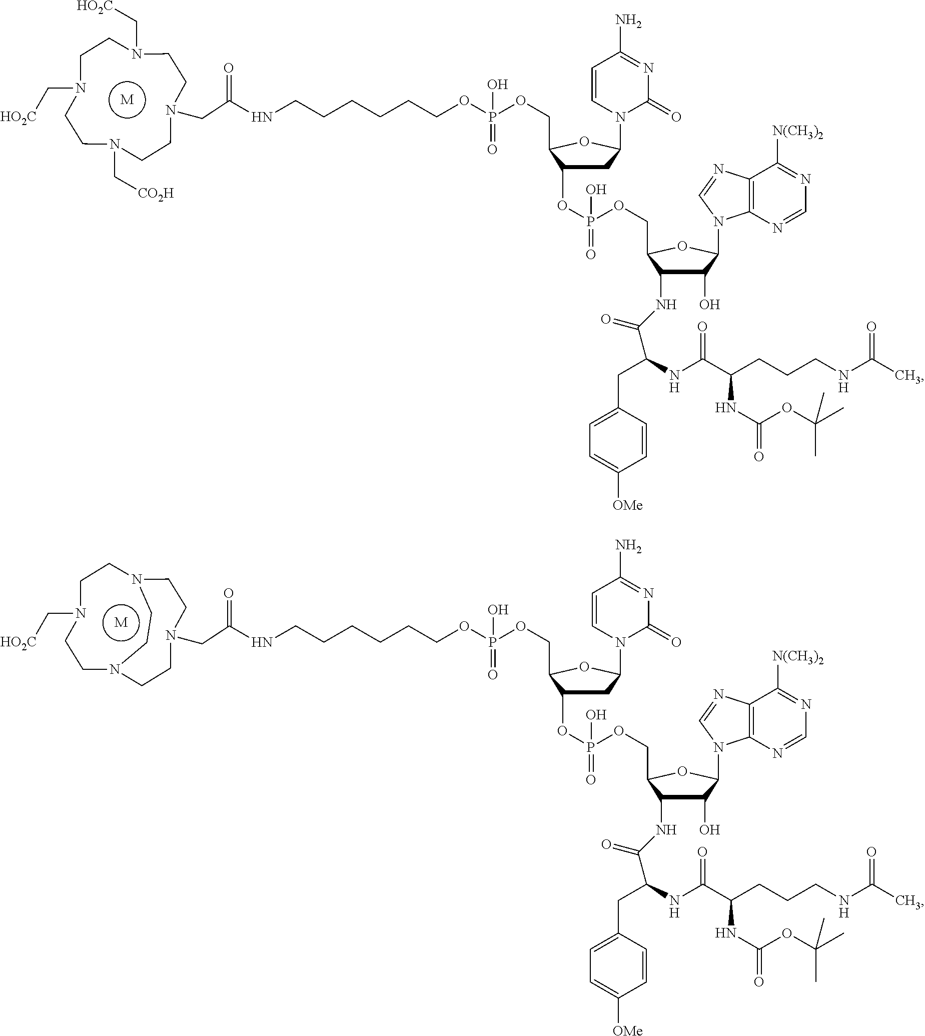

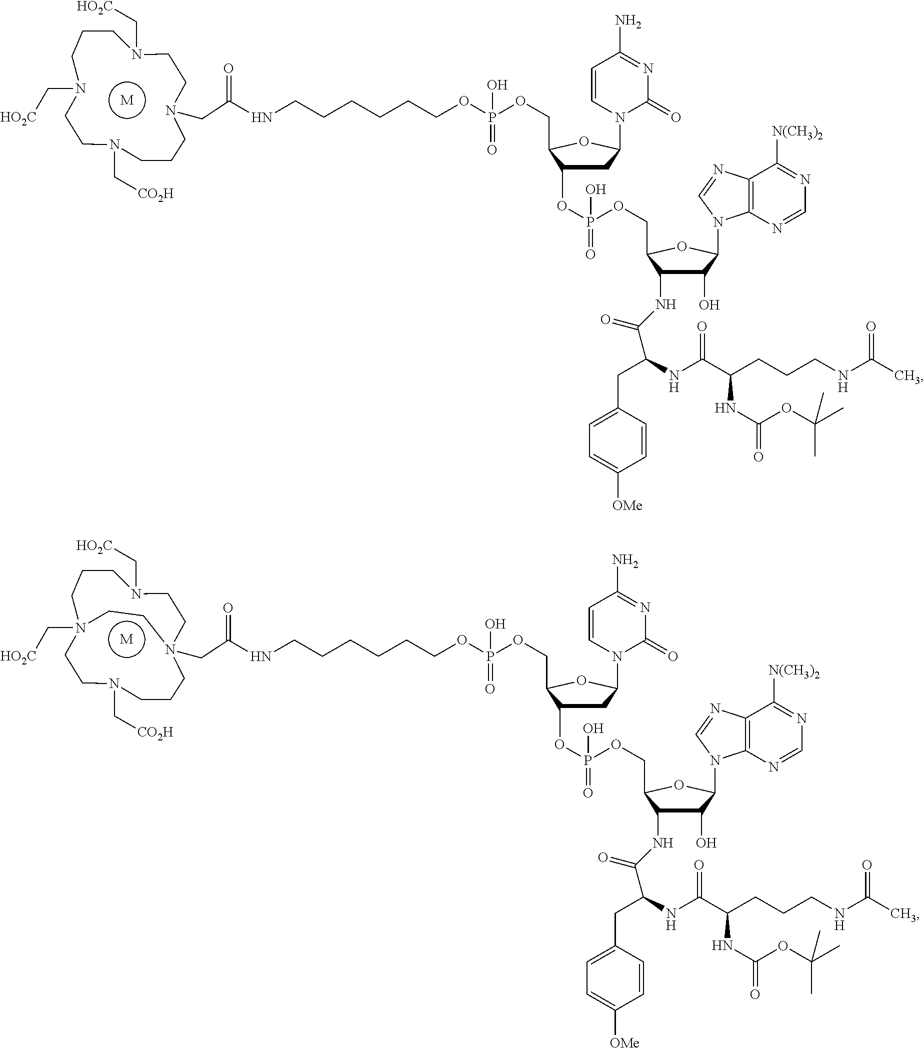

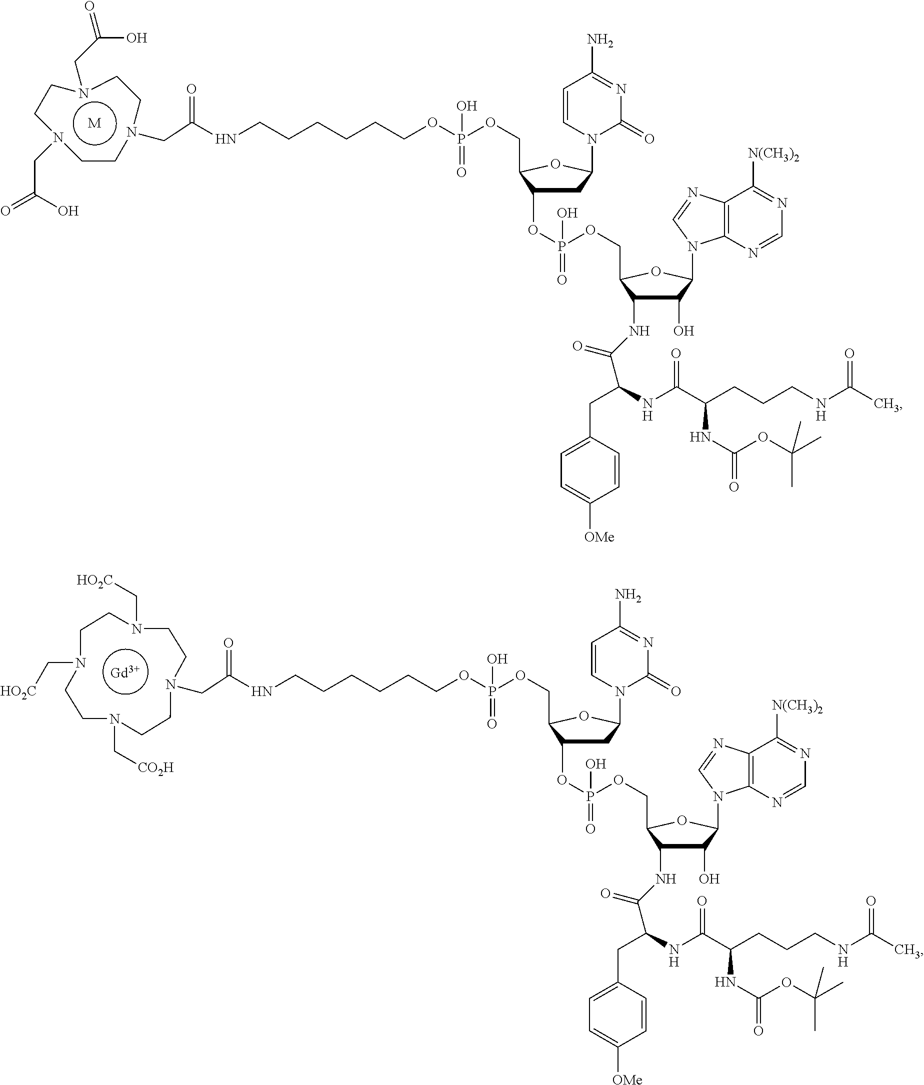

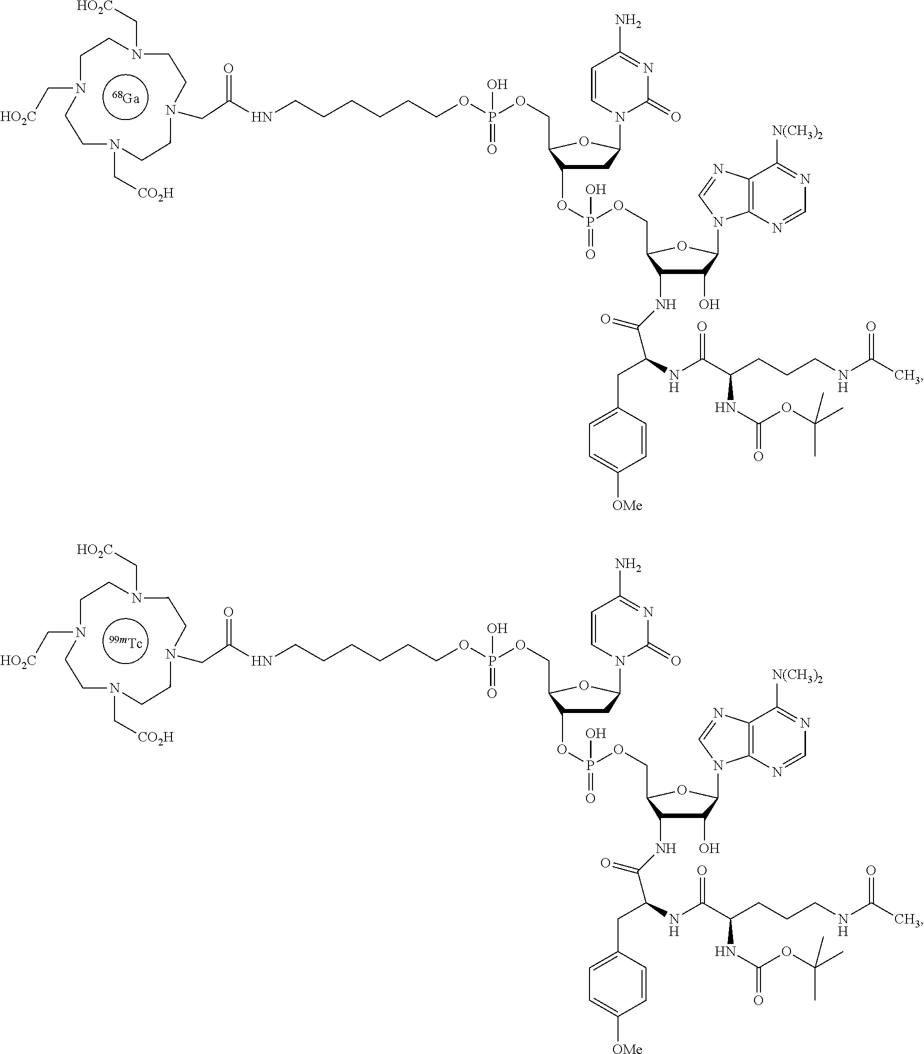

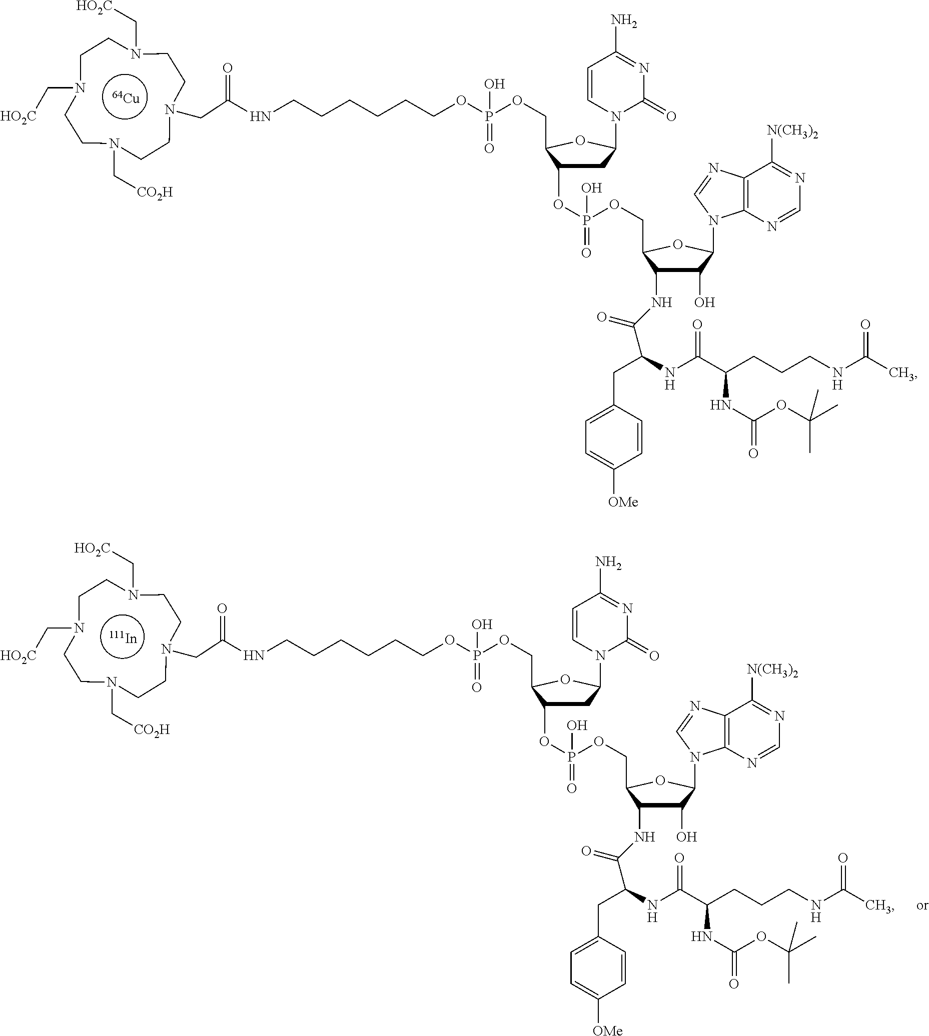

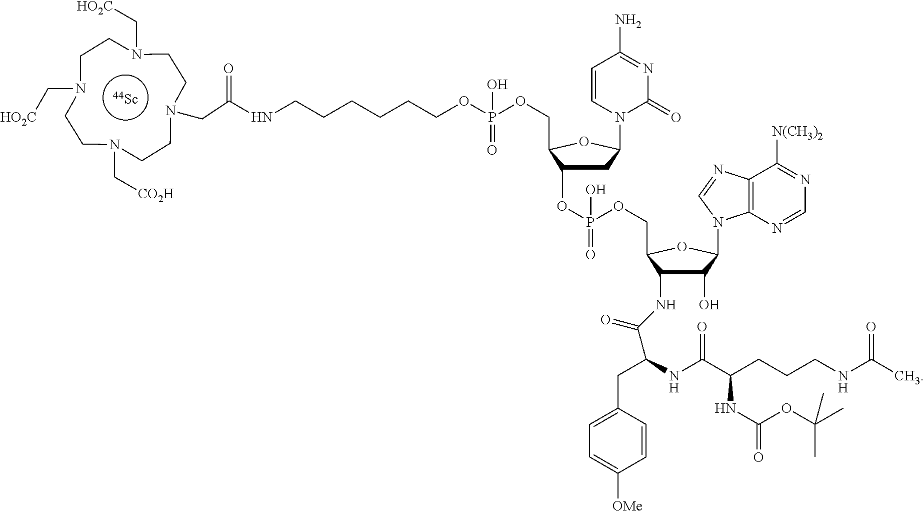

56.-57. (canceled)

58. The compound of claim 30 having the structure:

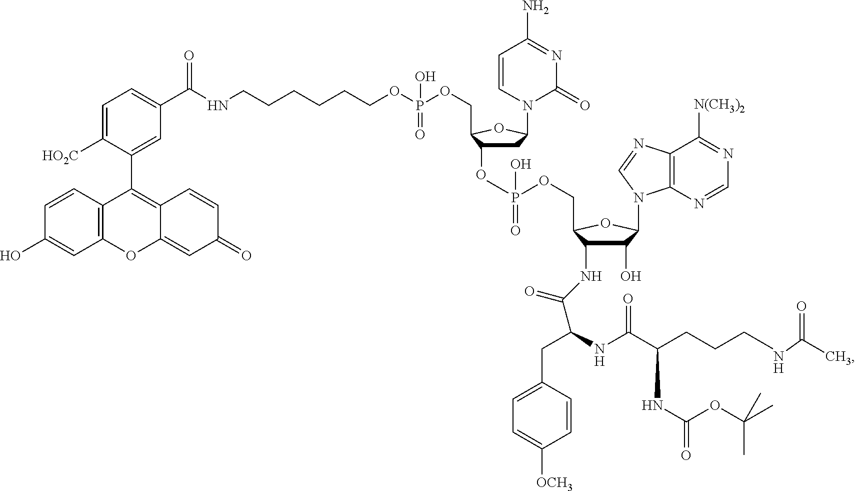

59. The compound of claim 29 having the structure:

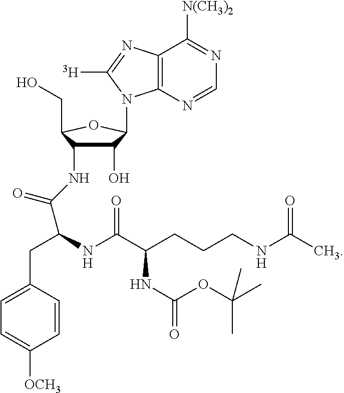

60. A pharmaceutical composition comprising the compound of claim 6 and a pharmaceutically acceptable carrier.

61. A method for detecting cancer cells in a subject comprising administering an effective amount of the compound of claim 6 to the subject, and imaging the subject with a molecular imaging device to detect the compound or composition in the subject.

62.-66. (canceled)

67. A method of imaging cancer cells in a subject comprising:
1) administering to the subject an effective amount of a compound having the structure: 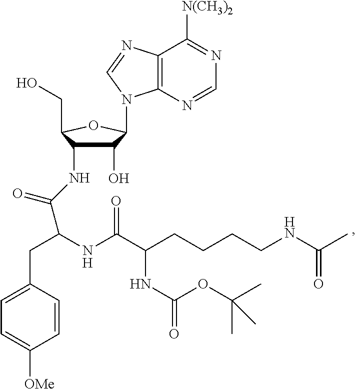
or a pharmaceutically acceptable salt thereof,
wherein the compound specifically accumulates at cancer cells in the subject and releases puromycin;
2) administering to the subject an amount of an antibody conjugated to a detectable marker, which antibody is capable of specifically binding to the puromycin at the cancer cells in the subject;
3) detecting in the subject the location of the detectable marker; and
4) obtaining an image of the cancer cells in the subject based on the location of the detectable marker in the subject.

68.-71. (canceled)
